(19) United States
(12) Patent Application Publication
(10) Pub. No.: US 2026/0138985 A1
(43) Pub. Date: May 21, 2026

(54) Azepino-Indoles for the Treatment of Neurological and Psychiatric Disorders

(71) Applicant: Terran Biosciences Inc., Miami Beach, FL (US)

(72) Inventors: Samuel CLARK, Miami Beach, FL (US); Matthew DUNCTON, Las Vegas, NV (US)

(21) Appl. No.: 19/451,295

(22) Filed: Jan. 16, 2026

Related U.S. Application Data
(63) Continuation of application No. 19/226,797, filed on Jun. 3, 2025, now abandoned.
Application No. 19/226,797 is a continuation of application No. 18/926,140, filed on Oct. 24, 2024, now abandoned.
Application No. 18/926,140 is a continuation of application No. 18/604,384, filed on Mar. 13, 2024, now abandoned.
Application No. 18/604,384 is a continuation of application No. 18/051,449, filed on Oct. 31, 2022, now abandoned.
(60) Provisional application No. 63/273,713, filed on Oct. 29, 2021.
(60) Provisional application No. 63/292,719, filed on Dec. 22, 2021.
(60) Provisional application No. 63/335,178, filed on Apr. 26, 2022.
(60) Provisional application No. 63/390,593, filed on Jul. 19, 2022.
(60) Provisional application No. 63/390,590, filed on Jul. 19, 2022.
(60) Provisional application No. 63/376,742, filed on Sep. 22, 2022.

(51) Int. Cl. C07D 487/04; A61P 25/28; C07D 471/14; C07D 487/14; C07D 491/147; C07F 7/08

(52) U.S. Cl. CPC C07D 487/04; A61P 25/28; C07D 471/14; C07D 487/14; C07D 491/147; C07F 7/0812; C07B 2200/05

(57) ABSTRACT

Disclosed herein are compounds of Formulae I or II,

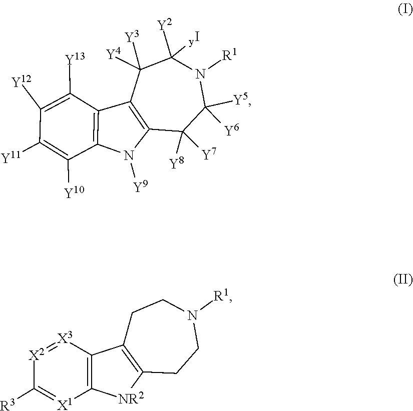

or an isotopically enriched compound thereof, or a pharmaceutically acceptable salt thereof, methods for making the compounds, and methods for their use.

### CROSS REFERENCE TO RELATED APPLICATIONS

The present application is a continuation of U.S. application Ser. No. 19/226,797, filed Jun. 3, 2025, which is a continuation of U.S. application Ser. No. 18/926,140, filed Oct. 24, 2024, which is a continuation of U.S. application Ser. No. 18/604,384, filed on Mar. 13, 2024, which is a continuation of U.S. application Ser. No. 18/051,449, filed Oct. 31, 2022, which claims priority to, and the benefit of, U.S. Provisional Application Nos. 63/273,713 filed Oct. 29, 2021, 63/292,719 filed Dec. 22, 2021, 63/335,178 filed Apr. 26, 2022, 63/390,590 filed Jul. 19, 2022, 63/390,593 filed Jul. 19, 2022 and 63/376,742 filed Sep. 22, 2022. The contents of the aforementioned patent applications are incorporated herein by reference in their entirety.

### FIELD

The present disclosure relates to azepino indole compounds and their use to treat brain and neurological disorders. The disclosure further relates to the provision of isotopically enriched compounds with improved characteristics.

### BACKGROUND

The psychedelic alkaloid ibogaine has robust anti-addictive properties in the clinic and in animal models. Ibogaine has the potential in treating patients addicted to a variety of substances, for example, opiates, psychostimulants, alcohol, and nicotine.

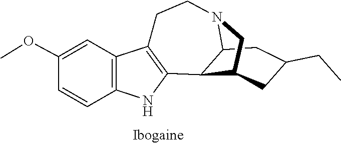 [SMILES: CCC1CC2[C@@H]3c4[nH]c5ccc(OC)cc5c4CC[N@@]2C[C@H]3C1]

Moreover, the therapeutic effects of ibogaine are long lasting, which has been attributed to its ability to modify addiction-related neural circuitry through activation of neurotrophic factor signaling. Ibogaine reduces symptoms of drug withdrawal, reduces drug cravings, and prevents relapse. In rodents, ibogaine reduces drug self-administration and prevents drug-induced dopamine release in several brain regions. However, several safety concerns have hindered the clinical development of ibogaine, including, for example, its toxicity, hallucinogenic potential, and proclivity for inducing cardiac arrhythmias via hERG channel inhibition.

Ibogaine increases glial cell line-derived neurotrophic factor (GDNF) expression in the ventral tegmental area (VTA), and intra-VTA infusion of ibogaine reduces alcohol-seeking behavior in rodents. Ibogaine impacts brain-derived neurotrophic factor (BDNF) and GDNF signaling in multiple brain regions implicated in the pathophysiology of addiction. Noribogaine, an active metabolite of ibogaine, is a potent psychoplastogen capable of increasing cortical neuron dendritic arbor complexity. Other psychoplastogens, such as lysergic acid diethylamide (LSD) and psilocin (the active metabolite of psilocybin) have anti-addictive properties in the clinic similar to ibogaine. The ability of psychoplastogens to promote structural and functional neural plasticity in addiction-related circuitry can explain their abilities to reduce drug-seeking behavior for weeks to months following a single administration. Moreover, by modifying neural circuitry rather than simply blocking the targets of a particular addictive substance (e.g., opioid receptors, nicotinic receptors, etc.), psychoplastogens like ibogaine have the potential to be broadly applicable anti-addictive agents.

However, ibogaine analogs, like many prospective drug candidates exhibit pharmacokinetic properties that undermine their use in clinical treatment. For example, such compounds may have undesirable absorption, distribution, metabolism and/or excretion (ADME) properties that prevent their wider use or limit their use in certain indications. While these compounds are useful in a variety of in vitro and in vivo contexts, there remains a need for compounds with improved effects and increased duration of actions. Compounds with such improved characteristics are disclosed herein.

### SUMMARY

The present disclosure relates to azepino-indole compounds for the treatment of neurological and psychiatric disorders. In one embodiment the compounds have improved efficacy, improved pharmacokinetic properties or both. In one embodiment the disclosed compounds are isotopically enriched at one or more position.

In one aspect of the disclosed embodiments the compounds are represented by Formula I

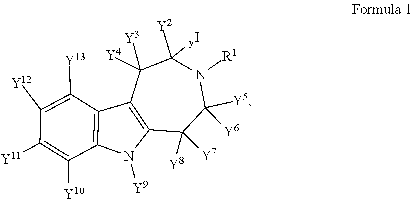

or
an enantiomer, a diastereomer, an isotopic derivative, or a pharmaceutically acceptable salt thereof, wherein:
R¹ is selected from hydrogen, deuterium, and C₁₋₆ alkyl;
Y¹, Y², Y³, Y⁴, Y⁵, Y⁶, Y⁷ and Y⁸ are each independently selected from hydrogen, deuterium, and C₁₋₆ alkyl;
Y⁹ is selected from hydrogen, deuterium, C₁₋₆ alkyl, C₃₋₈ cycloalkyl and C₄₋₁₄ alkyl-cycloalkyl and C₁₋₆ haloalkyl;
Y¹⁰, Y¹¹, Y¹² and Y¹³ are independently selected from hydrogen, deuterium, C₁₋₆ alkyl, C₂₋₆ alkenyl, C₂₋₆ alkynyl, halogen, C₁₋₆ haloalkyl, C₁₋₆ alkylamine, C₁₋₆ alkoxy, C₁₋₆ haloalkoxy, —OR², —NO₂, —CN, —C(O)R^{b}, —C(O)OR^{b}, —OC(O)R^{b}, —OC(O)OR^{b}, —N(R^{c}R^{c}), —N(R^{b})C(O)R^{b}, —C(O)N(R^{c}R^{c}), —N(R^{b})C(O)OR^{c}, —OC(O)N(R^{c}R^{c}), —N(R^{b})C(O)N(R^{c}R^{c}), —C(O)C(O)N(R^{c}R^{c}), —S(O₂)R^{b}, —S(O)₂N(R^{c}R^{c}), C₃₋₈ cycloalkyl, C₃₋₁₄ alkyl-cycloalkyl, C₄₋₁₀ heterocycloalkyl, C₄₋₁₆ alkyl-heterocycloalkyl, C₆₋₁₂ aryl, C₇₋₁₈ alkyl-aryl, C₅₋₁₀ heteroaryl, and C₄₋₁₆ alkyl-heteroaryl; R² is C₁₋₆ alkyl, C₃₋₈ cycloalkyl, C₃₋₈ cycloalkyl, C₃₋₁₄ alkyl-cycloalkyl, C₁₋₆ haloalkyl, C₄₋₁₀ heterocycloalkyl, C₄₋₁₆ alkyl-heterocycloalkyl, C₆₋₁₂ aryl, C₇₋₁₈ alkyl-aryl, C₅₋₁₀ heteroaryl, or C₄₋₁₆ alkyl-heteroaryl;
R^{b} is, for each occurrence, independently hydrogen, deuterium, or C₁₋₆ alkyl; and
R^{c} is, for each occurrence, selected from hydrogen, deuterium, C₁₋₆ alkyl, C₃₋₈ cycloalkyl, and C₄₋₁₄ alkyl-cycloalkyl, or two R^{c} together with the nitrogen to which they are attached to form a C₂₋₁₂ heterocycloalkyl.

In another aspect, the compounds have Formula II

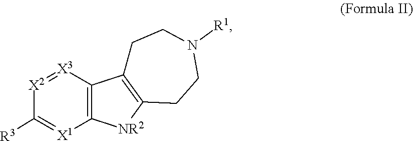

an enantiomer, a diastereomer, an isotopic derivative, or a pharmaceutically acceptable salt thereof, wherein
R¹ and R² independently are selected from hydrogen, deuterium, C₁₋₆ alkyl, —C(O)OR^{a}, —C(O)R^{a}, C₃₋₆ cycloalkyl, and —C(O)NR^{b}R^{b}, wherein alkyl is optionally substituted by one or more —S(O)₂R^{d}, —NR^{b}R^{b}, —OH, or —OD;
X¹ is C(R^{X1}) or N;
X² is C(R^{X2}) or N;
X³ is C(R^{X3}) or N;
R^{X1} is selected from hydrogen, deuterium, and alkyl, or together with R³ forms a heterocyclyl;
R^{X2} is selected from hydrogen, deuterium, halogen, C₁₋₆ alkyl, C₁₋₆ haloalkyl, C₃₋₆ cycloalkyl, —OR^{c} or together with R³ forms a heterocyclyl;
R^{X3} is selected from hydrogen, deuterium, halogen, C₁₋₆ alkyl, C₁₋₆ haloalkyl, C₃₋₆ cycloalkyl, —OR^{c}, —SR^{c}, —SF₅, and —CN;
R³ is selected from halo, C₁₋₆ alkyl, C₁₋₆ haloalkyl, C₃₋₆ cycloalkyl, —OR^{c}, —SR^{c}, —S(O)R^{d}, —S(O)₂R^{d}, —Si(R^{a})₃, —SF₅, and —CN or R³ together with R^{X1} or R^{X2} forms a heterocyclyl;
R^{a} is, for each occurrence, independently selected from the group consisting of C₁₋₆ alkyl, C₁₋₆ haloalkyl, and C₃₋₆ cycloalkyl;
R^{b} is, for each occurrence, independently selected from hydrogen, deuterium, C₁₋₆ alkyl, and C₃₋₆ cycloalkyl, or two R^{b}, together with the nitrogen atom to which they are attached, form a heterocyclylalkyl;
R^{c} is selected from the group consisting of C₁₋₆ alkyl, C₁₋₆ haloalkyl, and C₃₋₆ cycloalkyl; and
R^{d} is selected from the group consisting of C₁₋₆ alkyl, C₁₋₆ haloalkyl, C₃₋₆ cycloalkyl, and —NR^{b}R^{b}; provided that
when R^{X2} is selected from hydrogen, deuterium, and fluoro, R³ is not chloro, cyclopropyl, —S(O)₂CF₃, —SMe, —SEt, —S-iPr, —SCF₃, or —OMe; and
when X² is N, R³ is not —OMe.

In yet another aspect, a compound of formulas I or II is present in an enantiomeric or diastereomeric form, an isomer derivative or a mixture thereof.

In yet another aspect, at least one atom of Formula I or II is optionally enriched in an isotope.

In yet another aspect, a compound of Formula I or II is enriched in deuterium, tritium, carbon-14 or a combination thereof.

Also disclosed are methods for making the compounds and methods for their use.

The foregoing and other objects, features, and advantages of the invention will become more apparent from the following detailed description.

### BRIEF DESCRIPTION OF THE FIGURES

FIG. 1 shows control dose response curves for the selected GPCR Biosensor Assays.

FIG. 2 illustrates the effect of tabernanthalog, five representative compounds of the application, and psilocybin on average cumulative head twitches in mice.

FIG. 3 provides a bar chart of average total head twitches induced after administration of tabernanthalog, five representative compounds of the application, and psilocybin.

### DETAILED DESCRIPTION

### I. General

Disclosed herein are azepino-indole compounds, in particular, isotopically labeled azepino indoles, or isotopologues. The presently disclosed compounds, isotopically labeled azepino indoles, or isotopologues are useful for the treatment of a variety of brain disorders and other conditions. Without limitation to any particular theory, it is believed that the present compounds increase neuronal plasticity, and increase at least one of translation, transcription, or secretion of neurotrophic factors.

In one embodiment, by virtue of their isotopic enrichment, the presently disclosed compounds have improved pharmacokinetic and pharmacodynamic properties as compared to previously disclosed molecules. In certain embodiments the isotopic labels of the present compounds allow monitoring of its pharmacodynamic and ADME behavior following in vivo administration. In some embodiments, the isotopically enriched compounds described herein provide better therapeutic potential for neurological diseases than known compounds.

### Terms and Abbreviations

The term “isotopic enrichment factor” as used herein means the ratio between the isotopic abundance and the natural abundance of a specified isotope. It will be recognized that some variation of natural isotopic abundance occurs in a synthesized compound depending upon the origin of chemical materials used in the synthesis. Thus, a preparation of any compound will inherently contain small amounts of isotopologues, including deuterated isotopologues. The concentration of naturally abundant stable hydrogen isotopes, notwithstanding this variation, is small and immaterial as compared to the degree of stable isotopic substitution of compounds of this disclosure. In a compound of this disclosure, when a particular position is designated as having a particular isotope, such as deuterium, it is understood that the abundance of deuterium at that position is substantially greater than the natural abundance of deuterium, which is about 0.015% (on a mol/mol basis). A position designated as a particular isotope will have a minimum isotopic enrichment factor of at least 3000 (45% incorporation of the indicated isotope). Thus, isotopically enriched compounds disclosed herein having deuterium will have a minimum isotopic enrichment factor of at least 3000 (45% deuterium incorporation) at each atom designated as deuterium in the compound. Such compounds may be referred to herein as “deuterated” compounds.

In other embodiments, disclosed compounds have an isotopic enrichment factor for each designated atom of at least 3500 (52.5%). For example, for such disclosed compounds that are deuterium isotopologues, the compounds have an isotopic enrichment factor for each designated hydrogen atom of at least 3500 (52.5% deuterium incorporation at each designated atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). Compounds with a deuterium enrichment factor of at least about 3500 are referred to herein as “deuterated” compounds.

In the compounds of this disclosure any atom not specifically designated as a particular isotope is meant to represent any stable isotope of that atom. Unless otherwise stated, when a position is designated specifically as “H” or “protium”, the position is understood to have hydrogen at about its natural abundance isotopic composition.

The term “isotopologue” refers to a species that has the same chemical structure and formula as another compound, with the exception of the isotopic composition at one or more positions, e.g., H vs. D. Thus, isotopologues differ in their isotopic composition.

Abbreviations used: DMT, A,A-dimethyltryptamine; PFC, prefrontal cortex; 5-HT_{2A}, serotonin 2A; MPO, multiparameter optimization; LSD, lysergic acid diethylamide: TPSA, total polar surface area; MAP2, microtubule-associated protein 2; Nₘₐₓ, maximum number of crossings; 5-HT2B, serotonin 2B; DJV, days in vitro; VEH, vehicle; KET, ketamine; SEM, standard error of the mean; ANOVA, analysis of variance; DOM, 2,5-dimethoxy-4-methylamphetamine; OMe, methoxy; OBn, benzyloxy; F, fluoro; μM, micromolar; nM, nanomolar; pM, picomolar; V, vehicle; K, ketamine; ATR, attenuated total reflectance; FT-IR, Fourier transform infrared spectroscopy; UHPLC, ultra-high performance liquid chromatography; LRMS, low-resolution mass spectrometry; BSA, bovine serum albumin; DPBS, Dulbecco's phosphate-buffered saline; mTOR, mammalian target of rapamycin; AMP A, α-amino-3-hydroxy-5-methyl-4-isoxazolepropionic acid; TrkB, tropomyosin receptor kinase B; HTR, head-twitch response.

Certain chemical abbreviations used herein include: H=hydrogen, D=deuterium, Me=methyl or CH₃, Et=Ethyl or CH₂CH₃, ⁿPr=n-propyl or CH₂CH₂CH₃, ⁱPr=isopropyl or CH(CH₃)₂, ^{c}Pr=cyclopropyl, ⁿBu=n-butyl or CH₂CH₂CH₂CH₃, CCH=ethynyl, CH═CH₂=vinyl, and CH₂CH═CH₂=allyl.

The term “alkyl” refers to a straight or branched, saturated, aliphatic radical having the number of carbon atoms indicated. Alkyl can include any number of carbons, such as C₁₋₂, C₁₋₃, C₁₋₄, C₁₋₅, C₁₋₆, C₁₋₇, C₁₋₈, C₁₋₉, C₁₋₁₀, C₂₋₃, C₂₋₄, C₂₋₅, C₂₋₆, C₃₋₄, C₃₋₅, C₃₋₆, C₄₋₅, C₄₋₆ and C₅₋₆. For example, C₁₋₆ alkyl includes, but is not limited to, methyl, ethyl, propyl, isopropyl, butyl, isobutyl, sec-butyl, tert-butyl, pentyl, isopentyl, hexyl, and the like. Alkyl can also refer to alkyl groups having up to 20 carbon atoms, such as, but not limited to heptyl, octyl, nonyl, decyl and the like. Alkyl groups can be substituted or unsubstituted.

The term “alkylene” refers to a straight or branched, saturated, aliphatic radical having the number of carbon atoms indicated, and linking at least two other groups, i.e., a divalent hydrocarbon radical. The two moieties linked to the alkylene can be linked to the same atom or different atoms of the alkylene group. For instance, a straight chain alkylene can be the bivalent radical of —(CH₂)ₙ— where n is 1, 2, 3, 4, 5 or 6. Representative alkylene groups include, but are not limited to, methylene, ethylene, propylene, isopropylene, butylene, isobutylene, sec-butylene, pentylene and hexylene. Alkylene groups can be substituted or unsubstituted.

The term “alkenyl” refers to a straight chain or branched hydrocarbon having at least 2 carbon atoms and at least one double bond. Alkenyl can include any number of carbons, such as C₂, C₂₋₃, C₂₋₄, C₂₋₅, C₂₋₆, C₂₋₇, C₂₋₈, C₂₋₉, C₂₋₁₀, C₃, C₃₋₄, C₃₋₅, C₃₋₆, C₄, C₄₋₅, C₄₋₆, C₅, C₅₋₆, and C₆. Alkenyl groups can have any suitable number of double bonds, including, but not limited to, 1, 2, 3, 4, 5 or more. Examples of alkenyl groups include, but are not limited to, vinyl (ethenyl), propenyl, isopropenyl, 1-butenyl, 2-butenyl, isobutenyl, butadienyl, 1-pentenyl, 2-pentenyl, isopentenyl, 1,3-pentadienyl, 1,4-pentadienyl, 1-hexenyl, 2-hexenyl, 3-hexenyl, 1,3-hexadienyl, 1,4-hexadienyl, 1,5-hexadienyl, 2,4-hexadienyl, or 1,3,5-hexatrienyl. Alkenyl groups can be substituted or unsubstituted.

The term “alkynyl” refers to either a straight chain or branched hydrocarbon having at least 2 carbon atoms and at least one triple bond. Alkynyl can include any number of carbons, such as C₂, C₂₋₃, C₂₋₄, C₂₋₅, C₂₋₆, C₂₋₇, C₂₋₈, C₂₋₉, C₂₋₁₀, C₃, C₃₋₄, C₃₋₅, C₃₋₆, C₄, C₄₋₅, C₄₋₆, C₅, C_{5- 6}, and C₆. Examples of alkynyl groups include, but are not limited to, acetylenyl, propynyl, 1-butynyl, 2-butynyl, butadiynyl, 1-pentynyl, 2-pentynyl, isopentynyl, 1,3-pentadiynyl, 1,4-pentadiynyl, 1-hexynyl, 2-hexynyl, 3-hexynyl, 1,3-hexadiynyl, 1,4-hexadiynyl, 1,5-hexadiynyl and the like. Alkynyl groups can be substituted or unsubstituted.

The term “cycloalkyl” refers to a saturated or partially unsaturated, monocyclic, bicyclic, fused bicyclic or bridged polycyclic ring assembly containing from 3 to 12 ring atoms, or the number of atoms indicated. Cycloalkyl can include any number of carbons, such as C₃₋₆, C₄₋₆, C₅₋₆, C₃₋₈, C₄₋₈, C₅₋₈, C₆₋₈, C₃₋₉, C₃₋₁₀, C₃₋₁₁, and C₃₋₁₂. Saturated monocyclic cycloalkyl rings include, for example, cyclopropyl, cyclobutyl, cyclopentyl, cyclohexyl, and cyclooctyl. Bicyclic compounds include spirocyclic compounds, fused bicyclic compounds and bridged bicyclic compounds. Saturated bicyclic and polycyclic cycloalkyl rings include, for example, norbornane, [2.2.2] bicyclooctane, decahydronaphthalene and adamantane. Cycloalkyl groups can also be partially unsaturated, having one or more double or triple bonds in the ring. Representative cycloalkyl groups that are partially unsaturated include, but are not limited to, cyclobutene, cyclopentene, cyclohexene, cyclohexadiene (1,3- and 1,4-isomers), cycloheptene, cycloheptadiene, cyclooctene, cyclooctadiene (1,3-, 1,4- and 1,5-isomers), norbornene, and norbornadiene. When cycloalkyl is a saturated monocyclic C₃₋₈ cycloalkyl, exemplary groups include, but are not limited to cyclopropyl, cyclobutyl, cyclopentyl, cyclohexyl, cycloheptyl and cyclooctyl. When cycloalkyl is a saturated monocyclic C₃₋₆ cycloalkyl, exemplary groups include, but are not limited to cyclopropyl, cyclobutyl, cyclopentyl, and cyclohexyl. Cycloalkyl groups can be substituted or unsubstituted.

The term “alkyl-cycloalkyl” refers to a radical having an alkyl component and a cycloalkyl component, where the alkyl component links the cycloalkyl component to the point of attachment. The alkyl component is as defined above, except that the alkyl component is at least divalent, an alkylene, to link to the cycloalkyl component and to the point of attachment. The alkyl component can include any number of carbons, such as C₁₋₆, C₁₋₂, C₁₋₃, C₁₋₄, C₁₋₅, C₃₋₆, C₂₋₄, C₂₋₅, C₂₋₆, C₃₋₄, C₃₋₅, C₃₋₆, C₄₋₅, C₄₋₆ and C₅₋₆. The cycloalkyl component is as defined within. Exemplary alkyl-cycloalkyl groups include, but are not limited to, methyl-cyclopropyl, methyl-cyclobutyl, methyl-cyclopentyl and methyl-cyclohexyl.

The term “heterocyclyl” refers to heteroaryl and heterocyclylalkyl ring systems.

The term “heterocycloalkyl” refers to a cycloalkyl as defined above, having from 3 to 12 ring members wherein at least one carbon is replaced by a heteroatom selected from N, O and S. Heterocycloalkyl groups contain between 1 and 4 heteroatoms, unless otherwise specified. Heterocycloalkyl includes bicyclic compounds which include a heteroatom. The term “bicyclic compounds” includes spirocyclic compounds, fused bicyclic compounds, and bridged bicyclic compounds. The heteroatoms can also be oxidized, such as, but not limited to, —S(O)— and —S(O)₂—. Heterocycloalkyl groups can include any number of ring atoms, such as, 3 to 6, 4 to 6, 5 to 6, 3 to 8, 4 to 8, 5 to 8, 6 to 8, 3 to 9, 3 to 10, 3 to 11, or 3 to 12 ring members. Any suitable number of heteroatoms can be included in the heterocycloalkyl groups, such as 1, 2, 3, or 4, or 1 to 2, 1 to 3, 1 to 4, 2 to 3, 2 to 4, or 3 to 4. The heterocycloalkyl group can include groups such as aziridine, azetidine, pyrrolidine, piperidine, azepane, azocane, quinuclidine, pyrazolidine, imidazolidine, piperazine (1,2-, 1,3- and 1,4-isomers), oxirane, oxetane, tetrahydrofuran, oxane (tetrahydropyran), oxepane, thiirane, thietane, thiolane (tetrahydrothiophene), thiane (tetrahydrothiopyran), oxazolidine, isoxazolidine, thiazolidine, isothiazolidine, dioxolane, dithiolane, morpholine, thiomorpholine, dioxane, or dithiane. The heterocycloalkyl groups can also be fused to aromatic or non-aromatic ring systems to form members including, but not limited to, indoline. Heterocycloalkyl groups can be unsubstituted or substituted. For example, heterocycloalkyl groups can be substituted with C₁₋₆ alkyl or oxo (═O), among many others.

The term “alkyl-heterocycloalkyl” refers to a radical having an alkyl component and a heterocycloalkyl component, where the alkyl component links the heterocycloalkyl component to the point of attachment. The alkyl component is as defined above, except that the alkyl component is at least divalent, an alkylene, to link to the heterocycloalkyl component and to the point of attachment. The alkyl component can include any number of carbons, such as C₁₋₆, C₁₋₂, C₁₋₃, C₁₋₄, C₁₋₅, C₁₋₆, C₂₋₃, C₂₋₄, C₂₋₅, C₂₋₆, C₃₋₄, C₃₋₅, C₃₋₆, C₄₋₅, C₄₋₆ and C₅₋₆. The heterocycloalkyl component is as defined above. Alkyl-heterocycloalkyl groups can be substituted or unsubstituted.

The term “halogen” or “halo” refers to fluorine, chlorine, bromine and iodine or the corresponding fluoro, chloro, bromo and iodo radicals.

The term “haloalkyl” refers to alkyl, as defined above, where some or all of the hydrogen atoms are replaced with halogen atoms. As for alkyl group, haloalkyl groups can have any suitable number of carbon atoms, such as C₁₋₆. For example, haloalkyl includes trifluoromethyl, fluoromethyl, and the like. In some instances, the term “perfluoro” or “perhalo” can be used to define a compound or radical where all the hydrogens are replaced with fluorine or another halogen. For example, perfluoromethyl refers to 1,1,1-trifluoromethyl or —CF₃ and perfluoroethyl refers to —CF₂CF₃.

The term “alkoxy” refers to an alkyl group having an oxygen atom that connects the alkyl group to the point of attachment: alkyl-O—. As for alkyl group, alkoxy groups can have any suitable number of carbon atoms, such as C₁₋₆. Alkoxy groups include, for example, methoxy, ethoxy, propoxy, iso-propoxy, butoxy, 2-butoxy, iso-butoxy, sec-butoxy, tert-butoxy, pentoxy, hexoxy and the like. The alkoxy groups can be further substituted with a variety of substituents described within. Alkoxy groups can be substituted or unsubstituted.

The term “haloalkoxy” refers to an alkoxy group where some or all of the hydrogen atoms are substituted with halogen atoms. As for an alkyl group, haloalkoxy groups can have any suitable number of carbon atoms, such as C₁₋₆. The alkoxy groups can be substituted with 1, 2, 3, or more halogens. When all the hydrogens are replaced with a halogen, for example by fluorine, the compounds are per-substituted, for example, perfluorinated. Haloalkoxy includes, but is not limited to, trifluoromethoxy, 2,2,2,-trifluoroethoxy, perfluoroethoxy, and the like.

The term “amine” refers to an —N(R)₂ group where the R groups can be hydrogen, alkyl, alkenyl, alkynyl, cycloalkyl, heterocycloalkyl, aryl, or heteroaryl, among others. The R groups can be the same or different. The amino groups can be primary (each R is hydrogen), secondary (one R is hydrogen) or tertiary (each R is other than hydrogen).

The term “alkyl amine” refers to an alkyl group as defined within, having one or more amino groups. The amino groups can be primary, secondary or tertiary. The alkyl amine can be further substituted with a hydroxy group to form an amino-hydroxy group. Alkyl amines useful in the present invention include, but are not limited to, ethyl amine, propyl amine, isopropyl amine, ethylene diamine and ethanolamine. The amino group can link the alkyl amine to the point of attachment with the rest of the compound, be at the omega position of the alkyl group, or link together at least two carbon atoms of the alkyl group. One of skill in the art will appreciate that other alkyl amines are useful in the present invention.

The term “aryl” refers to an aromatic ring system having any suitable number of ring atoms and any suitable number of rings. Aryl groups can include any suitable number of ring atoms, such as, 6, 7, 8, 9, 10, 11, 12, 13, 14, 15 or 16 ring atoms, as well as from 6 to 10, 6 to 12, or 6 to 14 ring members. Aryl groups can be monocyclic, fused to form bicyclic or tricyclic groups, or linked by a bond to form a biaryl group. Representative aryl groups include phenyl, naphthyl and biphenyl. Other aryl groups include benzyl, having a methylene linking group. Some aryl groups have from 6 to 12 ring members, such as phenyl, naphthyl or biphenyl. Other aryl groups have from 6 to 10 ring members, such as phenyl or naphthyl. Some other aryl groups have 6 ring members, such as phenyl. Aryl groups can be substituted or unsubstituted.

The term “alkyl-aryl” refers to a radical having an alkyl component and an aryl component, where the alkyl component links the aryl component to the point of attachment. The alkyl component is as defined above, except that the alkyl component is at least divalent, an alkylene, to link to the aryl component and to the point of attachment. The alkyl component can include any number of carbons, such as C₁₋₆, C₁₋₂, C₁₋₃, C₁₋₄, C₁₋₅, C₁₋₆, C₂₋₃, C₂₋₄, C₂₋₅, C₂₋₆, C₁₋₄, C₃₋₅, C₃₋₆, C₄₋₅, C₄₋₆ and C₅₋₆. The aryl component is as defined above. Examples of alkyl-aryl groups include, but are not limited to, benzyl and ethyl-benzene. Alkyl-aryl groups can be substituted or unsubstituted.

The term “heteroaryl” refers to a monocyclic or fused bicyclic or tricyclic aromatic ring assembly containing 5 to 16 ring atoms, where from 1 to 5 of the ring atoms are a heteroatom such as N, O or S. Heteroaryl groups can include any number of ring atoms, such as, 5 to 6, 3 to 8, 4 to 8, 5 to 8, 6 to 8, 3 to 9, 3 to 10, 3 to 11, or 3 to 12 ring members. Any suitable number of heteroatoms can be included in the heteroaryl groups, such as 1, 2, 3, 4, or 5, or 1 to 2, 1 to 3, 1 to 4, 1 to 5, 2 to 3, 2 to 4, 2 to 5, 3 to 4, or 3 to 5. Heteroaryl groups can have from 5 to 8 ring members and from 1 to 4 heteroatoms, or from 5 to 8 ring members and from 1 to 3 heteroatoms, or from 5 to 6 ring members and from 1 to 4 heteroatoms, or from 5 to 6 ring members and from 1 to 3 heteroatoms. The heteroaryl group can include groups such as pyrrole, pyridine, imidazole, pyrazole, triazole, tetrazole, pyrazine, pyrimidine, pyridazine, triazine (1,2,3-, 1,2,4- and 1,3,5-isomers), thiophene, furan, thiazole, isothiazole, oxazole, and isoxazole. The heteroaryl groups can also be fused to aromatic ring systems, such as a phenyl ring, to form members including, but not limited to, benzopyrroles such as indole and isoindole, benzopyridines such as quinoline and isoquinoline, benzopyrazine (quinoxaline), benzopyrimidine (quinazoline), benzopyridazines such as phthalazine and cinnoline, benzothiophene, and benzofuran. Other heteroaryl groups include heteroaryl rings linked by a bond, such as bipyridine. Heteroaryl groups can be substituted or unsubstituted.

The term “alkyl-heteroaryl” refers to a radical having an alkyl component and a heteroaryl component, where the alkyl component links the heteroaryl component to the point of attachment. The alkyl component is as defined above, except that the alkyl component is at least divalent, an alkylene, to link to the heteroaryl component and to the point of attachment. The alkyl component can include any number of carbons, such as C₁₋₆, C₁₋₂, C₁₋₃, C₁₋₄, C₁₋₅, C₁₋₄, C₂₋₃, C₂₋₄, C₂₋₅, C₂₋₆, C₃₋₄, C₃₋₅, C₃₋₆, C₄₋₅, C₄₋₆ and C₅₋₆. The heteroaryl component is as defined within. Alkyl-heteroaryl groups can be substituted or unsubstituted.

The term “salt” refers to acid or base salts of the compounds disclosed herein, e.g., pharmaceutically acceptable salts. Illustrative examples of pharmaceutically acceptable salts are mineral acid (hydrochloric acid, hydrobromic acid, phosphoric acid, and the like) salts, organic acid (fumaric acid, acetic acid, propionic acid, glutamic acid, citric acid, tartaric acid and the like) salts, quaternary ammonium (methyl iodide, ethyl iodide, and the like) salts. It is understood that the pharmaceutically acceptable salts are non-toxic. Additional suitable pharmaceutically acceptable salts are known to those of skill in the art. See, e.g., Remington: The Science and Practice of Pharmacy, volume I and volume II. (22^{nd} Ed., University of the Sciences, Philadelphia), which is incorporated herein by reference.

The neutral forms of the compounds may be regenerated by contacting the salt with a base or acid and isolating the parent compound. The parent form of the compound differs from the various salt forms in certain physical properties, such as solubility in polar solvents, but otherwise the salts are equivalent to the parent form of the compound for the purposes of the present invention.
[1] “Pharmaceutically acceptable salt” refers to derivatives of the compounds of the present disclosure wherein the parent compound is modified by making acid or base salts thereof. Examples of pharmaceutically acceptable salts include, but are not limited to, mineral organic acid salts of basic residues such as amines, alkali organic salts of acidic residues such as carboxylic acids, and the like. The pharmaceutically acceptable salts include the conventional non-toxic salts or the quaternary ammonium salts of the parent compound formed, for example, from non-toxic inorganic organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicylic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc. In some embodiments, the pharmaceutically acceptable salt is a sodium salt, a potassium salt, a calcium salt, a magnesium salt, a diethylamine salt, a choline salt, a meglumine salt, a benzathine salt, a tromethamine salt, an ammonia salt, an arginine salt, or a lysine salt. [0084] Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present disclosure also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. In the salt form, it is understood that the ratio of the compound to the cation or anion of the salt can be 1:1, or any ratio other than 1:1, e.g., 3:1, 2:1, 1:2, or 1:3. It is to be understood that all references to pharmaceutically acceptable salts include solvent addition forms (solvates) or crystal forms (polymorphs) as defined herein, of the same salt. It is to be understood that the compounds of the present disclosure, for example, the salts of the compounds, can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.
[2] As used herein, the term “solvate” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

The term “pharmaceutically acceptable excipient” refers to a substance that aids the administration of an active agent to and absorption by a subject. Pharmaceutical excipients useful in the present invention include, but are not limited to, binders, fillers, disintegrants, lubricants, coatings, sweeteners, flavors and colors. One of skill in the art will recognize that other pharmaceutical excipients are useful in the present disclosure.

The term “pharmaceutically acceptable carrier, adjuvant, or vehicle” refers to a non-toxic carrier, adjuvant, or vehicle that does not destroy the pharmacological activity of the agent with which it is formulated. Pharmaceutically acceptable carriers, adjuvants or vehicles that may be used in the disclosed compositions include, but are not limited to, ion exchangers, alumina, stearates such as aluminum stearate, lecithin, serum proteins such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The term “composition” refers to a product comprising the specified ingredients in the specified amounts, as well as any product, which results, directly or indirectly, from combination of the specified ingredients in the specified amounts. By “pharmaceutically acceptable” it is meant the carrier, diluent or excipient must be compatible with the other ingredients of the formulation.

The term “isomers” refers to compounds with same chemical formula but different connectivity between the atoms in the molecule, leading to distinct chemical structures. Isomers include structural isomers and stereoisomers. Examples of structural isomers include, but are not limited to, tautomers and regioisomers. Examples of stereoisomers include but are not limited to diastereomers and enantiomers.

The term “administering” refers to any suitable mode of administration, including, oral administration, administration as a suppository, topical contact, parenteral, intravenous, intraperitoneal, intramuscular, intralesional, intranasal or subcutaneous administration, intrathecal administration, or the implantation of a slow-release device e.g., a mini-osmotic pump, to the subject.

The term “parenteral” as used herein includes subcutaneous, intravenous, intramuscular, intra-articular, intra-synovial, intrasternal, intrathecal, intrahepatic, intralesional and intracranial injection or infusion techniques.

As used herein, the term “treating” or “treat” describes the management and care of a patient for the purpose of combating a disease, condition, or disorder and includes the administration of a compound of the present disclosure, or a pharmaceutically acceptable salt, polymorph or solvate thereof, to alleviate the symptoms or complications of a disease, condition or disorder, or to eliminate the disease, condition or disorder. The term “treat” can also include treatment of a cell in vitro or an animal model. It is to be appreciated that references to “treating” or “treatment” include the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

The term “subject” refers to an animal, such as a mammal, including, but not limited to, primates (e.g., humans), cows, sheep, goats, horses, dogs, cats, rabbits, rats, mice and the like. In certain embodiments, the subject is a human subject.

The term “therapeutically effective amount” or “therapeutically sufficient amount” or “effective or sufficient amount” refers to a dose that produces therapeutic effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins). In sensitized cells, the therapeutically effective dose can often be lower than the conventional therapeutically effective dose for non-sensitized cells.

The term “neuronal plasticity” refers to the ability of the brain to change its structure and/or function continuously throughout a subject's life. Examples of the changes to the brain include, but are not limited to, the ability to adapt or respond to internal and/or external stimuli, such as due to an injury, and the ability to produce new neurites, dendritic spines, and synapses.

The term “brain disorder” refers to a neurological disorder which affects the brain's structure and function. Brain disorders can include, but are not limited to, Alzheimer's, Parkinson's disease, psychological disorder, depression, treatment resistant depression, addiction, anxiety, post-traumatic stress disorder, suicidal ideation, major depressive disorder, bipolar disorder, schizophrenia, stroke, traumatic brain injury, and substance use disorder.

The term “combination therapy” refers to a method of treating a disease or disorder, wherein two or more different pharmaceutical agents are administered in overlapping regimens so that the subject is simultaneously exposed to both agents. For example, the compounds of the invention can be used in combination with other pharmaceutically active compounds. The compounds of the invention can be administered simultaneously (as a single preparation or separate preparation) or sequentially to the other drug therapy. In general, a combination therapy envisions administration of two or more drugs during a single cycle or course of therapy.

The term “neurotrophic factors” refers to a family of soluble peptides or proteins which support the survival, growth, and differentiation of developing and mature neurons.

The term “modulate” or “modulating” or “modulation” refers to an increase or decrease in the amount, quality, or effect of a particular activity, function or molecule. By way of illustration and not limitation, agonists, partial agonists, antagonists, and allosteric modulators (e.g., a positive allosteric modulator) of a G protein-coupled receptor (e.g., 5HT_{2A}) are modulators of the receptor.

The term “agonism” refers to the activation of a receptor or enzyme by a modulator, or agonist, to produce a biological response.

The term “agonist” refers to a modulator that binds to a receptor or enzyme and activates the receptor to produce a biological response. By way of example only, “5HT_{2A} agonist” can be used to refer to a compound that exhibits an EC₅₀ with respect to 5HT_{2A} activity of no more than about 100 mM. In some embodiments, the term “agonist” includes full agonists or partial agonists. “Full agonist” refers to a modulator that binds to and activates a receptor with the maximum response that an agonist can elicit at the receptor. The term “partial agonist” refers to a modulator that binds to and activates a given receptor, but has partial efficacy, that is, less than the maximal response, at the receptor relative to a full agonist.

The term “positive allosteric modulator” refers to a modulator that binds to a site distinct from the orthosteric binding site and enhances or amplifies the effect of an agonist.

The term “antagonism” refers to the inactivation of a receptor or enzyme by a modulator, or antagonist. Antagonism of a receptor, for example, is when a molecule binds to the receptor and does not allow activity to occur.

The term “antagonist” or “neutral antagonist” refers to a modulator that binds to a receptor or enzyme and blocks a biological response. An antagonist has no activity in the absence of an agonist or inverse agonist but can block the activity of either, causing no change in the biological response.

### Compounds

Disclosed herein are azepine indole compounds that include the psychoplastogenic pharmacophore of ibogaine. Recently, researchers have reported some progress in developing compounds that maintain the ibogaine pharmacophore and potential therapeutic efficacy, but lack ibogaine's toxicity and hallucinogenic effects. For example, the compound tabernanthalog (TBG), a simplified analog of ibogaine (or the iboga alkaloid tabernanthine),

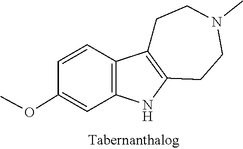

was reported to be non-hallucinogenic, but have 5-HT_{2A} activity.
(See, Dong et al., Cell, 184, 2779-2792; Olson et al., WO 2020/176599; Cameron, et al., Nature. 2021; 589(7842):474-479). Still, compounds such as tabernanthalog do not have the drug-like pharmacokinetic and pharmacodynamic properties to support their wider use in the clinical treatment of brain disorders.

### I. Isotopically Enriched Azepine Indoles

The present inventors observed that the metabolic properties of the disclosed azepine indoles could be improved by isotopic enrichment, in particular, deuterium or tritium enrichment. In this approach, one attempts to slow the CYP-mediated metabolism of a drug or to reduce the formation of undesirable metabolites by replacing one or more protium (¹H) atoms with deuterium atoms. Deuterium is a safe, stable, non-radioactive isotope of hydrogen. Compared to protium, deuterium forms stronger bonds with carbon. In select cases, the increased bond strength imparted by deuterium can positively affect the pharmacokinetic properties of a drug, creating the potential for improved drug efficacy, safety, and/or tolerability. At the same time, because the size and shape of deuterium are essentially identical to those of protium, replacement of protium by deuterium would not be expected to affect the biochemical potency and selectivity of the drug as compared to the original chemical entity that contains only hydrogen. Tritium, ³H, forms still stronger bonds with carbon than deuterium. Thus, replacement of protium with tritium also can affect the pharmacokinetic properties of a molecule. Moreover, tritium is a beta emitter, meaning that enriching a molecule with tritium allows determination of pharmacokinetic and pharmacodynamic properties of the molecule to better understand its activity and ADME properties.

Accordingly, in certain embodiments, the present invention provides a compound of Formula I:

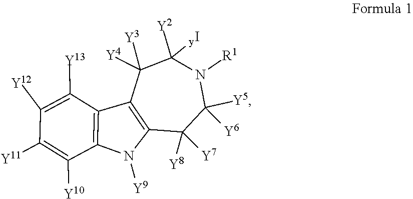

or
an enantiomer, a diastereomer, an isotopic derivative, or a pharmaceutically acceptable salt thereof, wherein:
R¹ is selected from hydrogen, deuterium, and C₁₋₆ alkyl;
Y¹, Y², Y³, Y⁴, Y⁵, Y⁶, Y⁷ and Y⁸ are each independently selected from hydrogen, deuterium, and C₁₋₆ alkyl;
Y⁹ is selected from hydrogen, deuterium, C₁₋₆ alkyl, C₃₋₈ cycloalkyl and C₄₋₁₄ alkyl-cycloalkyl and C₁₋₆ haloalkyl;
Y¹⁰, Y¹¹, Y¹² and Y¹³ are independently selected from hydrogen, deuterium, C₁₋₆ alkyl, C₂₋₆ alkenyl, C₂₋₆ alkynyl, halogen, C₁₋₆ haloalkyl, C₁₋₆ alkylamine, C₁₋₆ alkoxy, C₁₋₆ haloalkoxy, —OR², —NO₂, —CN, —C(O)R^{b}, —C(O)OR^{b}, —OC(O)R^{b}, —OC(O)OR^{b}, —N(R^{c}R^{c}), —N(R^{b})C(O)R^{b}, —C(O)N(R^{c}R^{c}), —N(R^{b})C(O)OR^{c}, —OC(O)N(R^{c}R^{c}), —N(R^{b})C(O)N(R^{c}R^{c}), —C(O)C(O)N(R^{c}R^{c}), —S(O₂)R^{b}, —S(O)₂N(R^{c}R^{c}), C₃₋₈ cycloalkyl, C₃₋₁₄ alkyl-cycloalkyl, C₄₋₁₀ heterocycloalkyl, C₄₋₁₆ alkyl-heterocycloalkyl, C₆₋₁₂ aryl, C₇₋₁₈ alkyl-aryl, C₅₋₁₀ heteroaryl, and C₄₋₁₆ alkyl-heteroaryl;
R² is C₁₋₆ alkyl, C₃₋₈ cycloalkyl, C₃₋₈ cycloalkyl, C₃₋₁₄ alkyl-cycloalkyl, C₁₋₆ haloalkyl, C₄₋₁₀ heterocycloalkyl, C₄₋₁₆ alkyl-heterocycloalkyl, C₆₋₁₂ aryl, C₇₋₁₈ alkyl-aryl, C₅₋₁₀ heteroaryl, or C₄₋₁₆ alkyl-heteroaryl;
R^{b} is, for each occurrence, independently hydrogen, deuterium, or C₁₋₆ alkyl; and
R^{c} is, for each occurrence, selected from hydrogen, deuterium, C₁₋₆ alkyl, C₃₋₈ cycloalkyl, and C₄₋₁₄ alkyl-cycloalkyl, or two R^{c} together with the nitrogen to which they are attached to form a C²⁻¹² heterocycloalkyl.

In embodiments of Formula I, Y¹¹ or Y¹² are selected from hydrogen, deuterium, C₁₋₆ alkoxy and —OR². In certain aspects of this embodiment, Y¹¹ is C₁₋₆ alkoxy or —OR². Particular embodiments of Formula I wherein Y¹¹ is C₁₋₆ alkoxy or —OR² have Formula I-1:

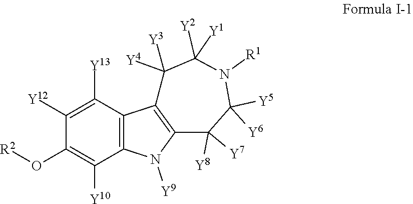

wherein R² is selected from C₁₋₆ alkyl; C₃₋₈ cycloalkyl and C₁₋₆ haloalkyl.

In additional embodiments of Formula I and Formula I-1, R¹ is C₁₋₆ alkyl, such as methyl. Such embodiments can be represented by Formula I-1A:

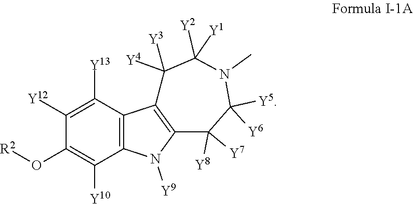

Further embodiments of Formula I and Formula I-1 have Y¹¹ C₁₋₆ alkoxy, such as methoxy, ethoxy or isopropoxy. Compounds wherein Y¹¹ is methoxy in Formula I or R² is methyl in Formula I-1 can be represented by Formula I-1B:

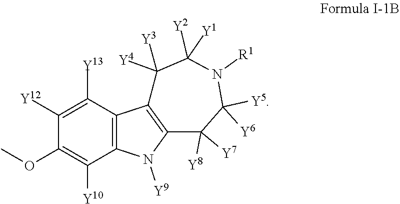

In embodiments of the compounds disclosed herein, including those represented by Formulas I, I-1, I-1A and I-1B, the compounds are enriched in deuterium, tritium, carbon-14 or a combination thereof.

In certain embodiments described above, at least one of Y¹, Y², Y³, Y⁴, Y⁵, Y⁶, Y⁷, Y⁸, Y⁹, Y¹⁰, Y¹¹, Y¹², Y¹³, R¹ and R² is enriched in deuterium.

In certain of the embodiments of Formulas I, I-1, I-1A and I-1B, described herein, at least one of Y¹, Y², Y³, Y⁴, Y⁵, Y⁶, Y⁷ and Y⁸ is enriched in deuterium.

In particular embodiments of the isotopically enriched compounds of Formulas I, I-1, I-1A and I-1B, the isotopically enriched compound have at least one of R¹ and R² enriched in deuterium.

In some embodiments of the isotopically enriched compounds of Formula I, at least one of Y¹, Y², Y³, Y⁴, Y⁵, Y⁶, Y⁷, Y⁸, Y⁹, Y¹⁰, Y¹¹, Y¹², Y¹³ and R¹ is enriched in deuterium.

In some embodiments of the isotopically enriched compounds of Formula I, R¹ is selected from CH₃, CH₂D, CHD₂ and CD₃ and optionally at least one of Y¹, Y², Y³, Y⁴, Y⁵, Y⁶, Y⁷, Y⁸, Y⁹, Y¹⁰, Y¹¹, Y¹² and Y¹³ is enriched in deuterium. In some embodiments of the isotopically enriched compounds of Formula I, R¹ is CH₂D, CHD₂ and CD₃ and at least one of Y¹, Y², Y³, Y⁴, Y⁵, Y⁶, Y⁷, Y⁸, Y⁹, Y¹⁰, Y¹¹, Y¹² and Y¹³ is enriched in deuterium.

In certain embodiments of the isotopically enriched compounds of Formula I, Y¹¹ is —OR² and both of R¹ and R² are methyl and optionally at least one of Y¹, Y², Y³, Y⁴, Y⁵, Y⁶, Y⁷, Y⁸, Y⁹, Y¹⁰, Y¹² and Y¹³ is enriched in deuterium. Thus, in certain examples of the isotopically enriched compounds Formula I disclosed herein, R¹ and R² are independently selected from CH₃, CH₂D, CHD₂ and CD₃ and optionally at least one of, Y¹, Y², Y³, Y⁴, Y⁵, Y⁶, Y⁷, Y⁸, Y⁹, Y¹⁰, Y¹² and Y¹³ is enriched in deuterium. In certain examples of the isotopically enriched compounds Formula I, R¹ is selected from CH₂D, CHD₂ and CD₃ and optionally at least one of Y¹, Y², Y³, Y⁴, Y⁵, Y⁶, Y⁷, Y⁸, Y⁹, Y¹⁰, Y¹² and Y¹³ is enriched in deuterium. Similarly, in certain examples of the isotopically enriched compounds Formula I, R² is selected from CH₂D, CHD₂ and CD₃ and optionally at least one of Y¹, Y², Y³, Y⁴, Y⁵, Y⁶, Y⁷, Y⁸, Y⁹, Y¹⁰, Y¹² and Y¹³ is enriched in deuterium.

In some embodiments of isotopically enriched compounds of Formula I-1, at least one of R¹ and R² is enriched in deuterium and optionally at least one of Y¹, Y², Y³, Y⁴, Y⁵, Y⁶, Y⁷, Y⁸, Y⁹, Y¹⁰, Y¹² and Y¹³ is enriched in deuterium. In some embodiments of isotopically enriched compounds of Formula I-1, R¹ and R² are independently selected from CH₃, CH₂D, CHD₂ and CD₃ and optionally at least one of Y¹, Y², Y³, Y⁴, Y⁵, Y⁶, Y⁷, Y⁸, Y⁹, Y¹⁰, Y¹² and Y¹³ is enriched in deuterium. In some embodiments of the isotopically enriched compounds of Formula I-1, R¹ and R² are independently selected from CH₂D, CHD₂ and CD₃ and optionally at least one of Y¹, Y², Y³, Y⁴, Y⁵, Y⁶, Y⁷, Y⁸, Y⁹, Y¹⁰, Y¹² and Y¹³ is enriched in deuterium.

In some embodiments of isotopically enriched compounds of Formula I-1, both of R¹ and R² are methyl. In some embodiments of isotopically enriched compounds of Formula I-1, R² is a methyl. In some embodiments of isotopically enriched compounds of Formula I-1, R¹ is a methyl.

In certain embodiments of the isotopically enriched compounds of Formula I-1, R¹ and R² are independently selected from CH₃, CH₂D, CHD₂ and CD₃. In certain embodiments of the isotopically enriched compounds of Formula I-1, R¹ is selected from CH₂D, CHD₂ and CD₃. In certain embodiments of the isotopically enriched compounds of Formula I-1, R² is selected from CH₂D, CHD₂ and CD₃.

In some embodiments of isotopically enriched compounds of Formula I-1, both of R¹ and R² are methyl and optionally at least one of Y¹, Y², Y³, Y⁴, Y⁵, Y⁶, Y⁷, Y⁸, Y⁹, Y¹⁰, Y¹² and Y¹³ is enriched in deuterium. In some embodiments of isotopically enriched compounds of Formula I-1, R² is a methyl and optionally at least one of Y¹, Y², Y³, Y⁴, Y⁵, Y⁶, Y⁷, Y⁸, Y⁹, Y¹⁰, Y¹² and Y¹³ is enriched in deuterium. In some embodiments of isotopically enriched compounds of Formula I-1, R¹ is a methyl and optionally at least one of Y¹, Y², Y³, Y⁴, Y⁵, Y⁶, Y⁷, Y⁸, Y⁹, Y¹⁰, Y¹² and Y¹³ is enriched in deuterium.

In certain embodiments of the isotopically enriched compounds of Formula I-1, R¹ and R² are independently selected from CH₃, CH₂D, CHD₂ and CD₃ and optionally at least one of Y¹, Y², Y³, Y⁴, Y⁵, Y⁶, Y⁷, Y⁸, Y⁹, Y¹⁰, Y¹² and Y¹³ is enriched in deuterium. In certain embodiments of the isotopically enriched compounds of Formula I-1, R¹ is selected from CH₂D, CHD₂ and CD₃ and optionally at least one of Y¹, Y², Y³, Y⁴, Y⁵, Y⁶, Y⁷, Y⁸, Y⁹, Y¹⁰, Y¹² and Y¹³ is enriched in deuterium. In certain embodiments of the isotopically enriched compounds of Formula I-1, R² is selected from CH₂D, CHD₂ and CD₃ and optionally at least one of Y¹, Y², Y³, Y⁴, Y⁵, Y⁶, Y⁷, Y⁵, Y⁹, Y¹⁰, Y¹² and Y¹³ is enriched in deuterium.

In some embodiments of isotopically enriched compounds of Formula I-1A, R² is enriched in deuterium and optionally at least one of Y¹, Y², Y³, Y⁴, Y⁵, Y⁶, Y⁷, Y⁸, Y⁹, Y¹⁰, Y¹² and Y¹³ is enriched in deuterium. In some embodiments of isotopically enriched compounds of Formula I-1A, R² is selected from CH₃, CH₂D, CHD₂ and CD₃ and optionally at least one of Y¹, Y², Y³, Y⁴, Y⁵, Y⁶, Y⁷, Y⁸, Y⁹, Y¹⁰, Y¹² and Y¹³ is enriched in deuterium. In some embodiments of the isotopically enriched compounds of Formula I-1A, R² is selected from CH₂D, CHD₂ and CD₃ and optionally at least one of Y¹, Y², Y³, Y⁴, Y⁵, Y⁶, Y⁷, Y⁸, Y⁹, Y¹⁰, Y¹² and Y¹³ is enriched in deuterium.

In some embodiments of isotopically enriched compounds of Formula I-1A, R² is a methyl. In some embodiments of isotopically enriched compounds of Formula I-1A, R² is selected from CH₃, CH₂D, CHD₂ and CD₃. In some embodiments of isotopically enriched compounds of Formula I-1A, R² is CH₂D, CHD₂ and CD₃.

In some embodiments of isotopically enriched compounds of Formula I-1B, R¹ is enriched in deuterium and optionally at least one of Y¹, Y², Y³, Y⁴, Y⁵, Y⁶, Y⁷, Y⁸, Y⁹, Y¹⁰, Y¹² and Y¹³ is enriched in deuterium. In some embodiments of isotopically enriched compounds of Formula I-1B, R¹ is selected from CH₃, CH₂D, CHD₂ and CD₃ and optionally at least one of Y¹, Y², Y³, Y⁴, Y⁵, Y⁶, Y⁷, Y⁸, Y⁹, Y¹⁰, Y¹² and Y¹³ is enriched in deuterium. In some embodiments of the isotopically enriched compounds of Formula I-1B, R¹ is selected from CH₂D, CHD₂ and CD₃ and optionally at least one of Y¹, Y², Y³, Y⁴, Y⁵, Y⁶, Y⁷, Y⁸, Y⁹, Y¹⁰, Y¹² and Y¹³ is enriched in deuterium.

In some embodiments of isotopically enriched compounds of Formula I-1B, R¹ is a methyl. In some embodiments of isotopically enriched compounds of Formula I-1B, R¹ is selected from CH₃, CH₂D, CHD₂ and CD₃. In some embodiments of the isotopically enriched compounds of Formula I-1B, R¹ is CH₂D, CHD₂ and CD₃.

In further embodiments of the isotopically enriched compounds of Formula I, Y¹⁰, Y¹¹, Y¹² and Y¹³ each independently are selected from protium and deuterium.

In further embodiments of the isotopically enriched compounds of Formulas I-1, I-1A and I-1B, Y¹⁰, Y¹² and Y¹³ each independently are selected from protium and deuterium.

More particular embodiments of the disclosed isotopically enriched compounds of Formulas I, I-1, I-1A and I-1B have the formulas illustrated below:

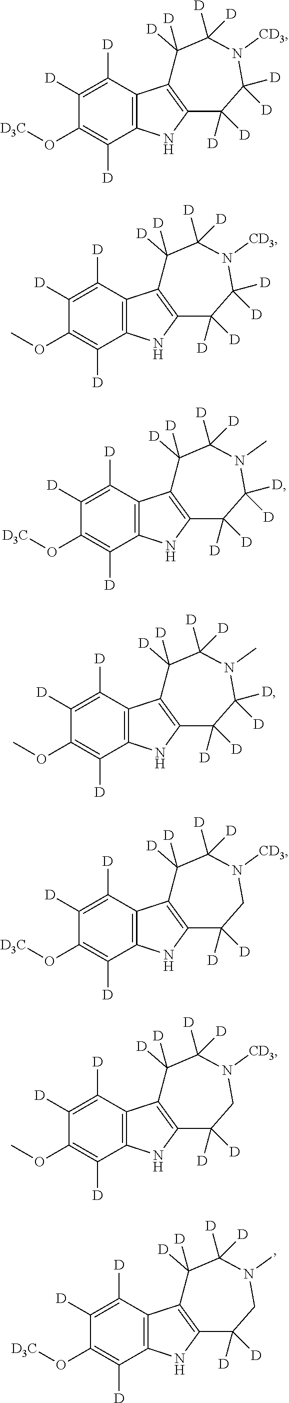

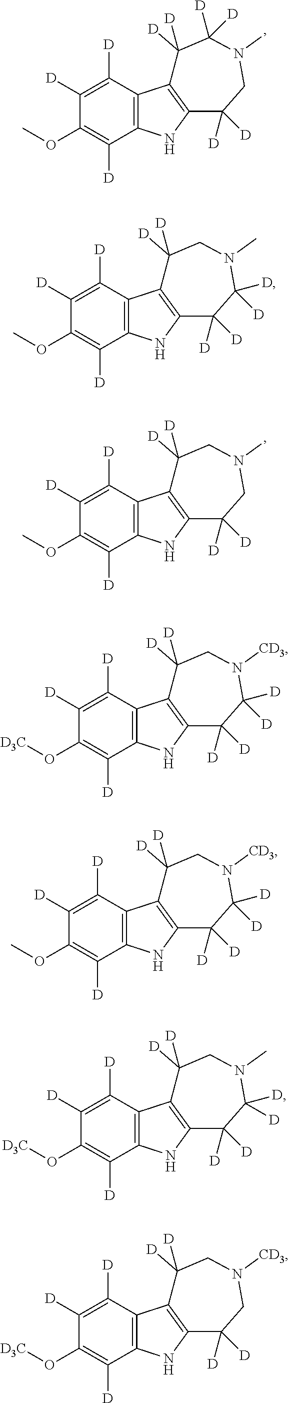

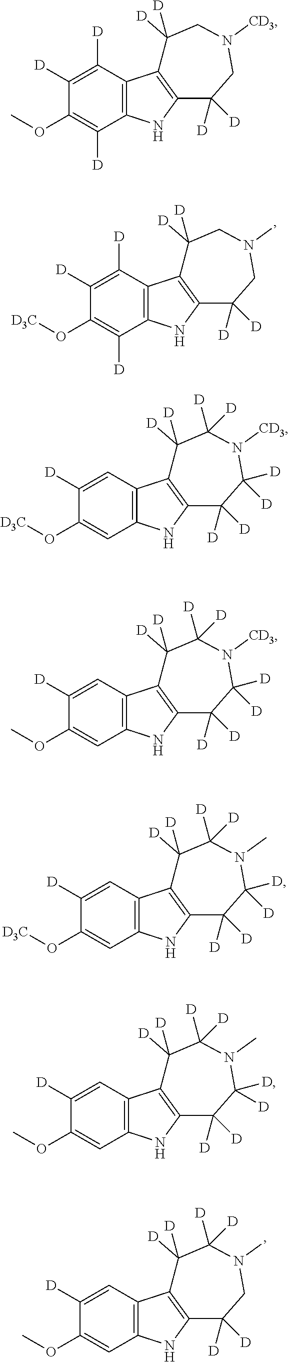

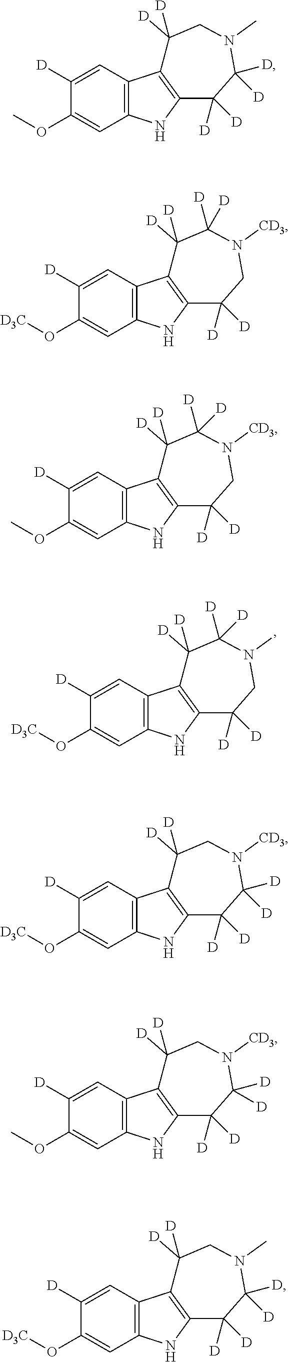

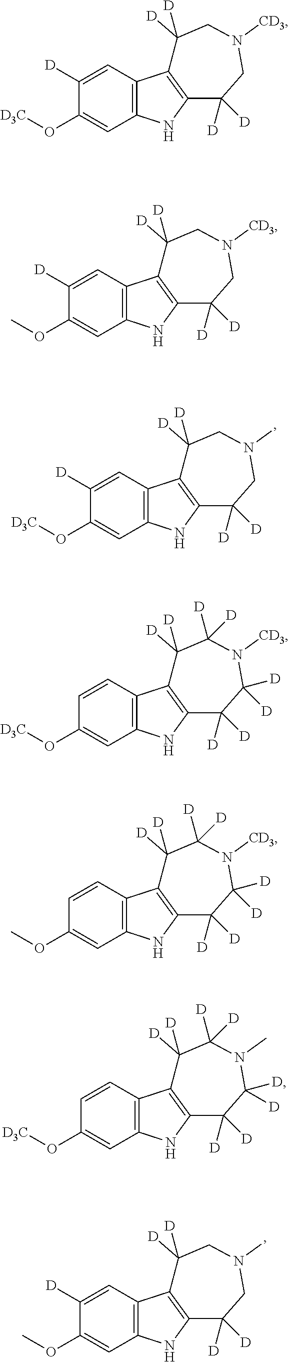

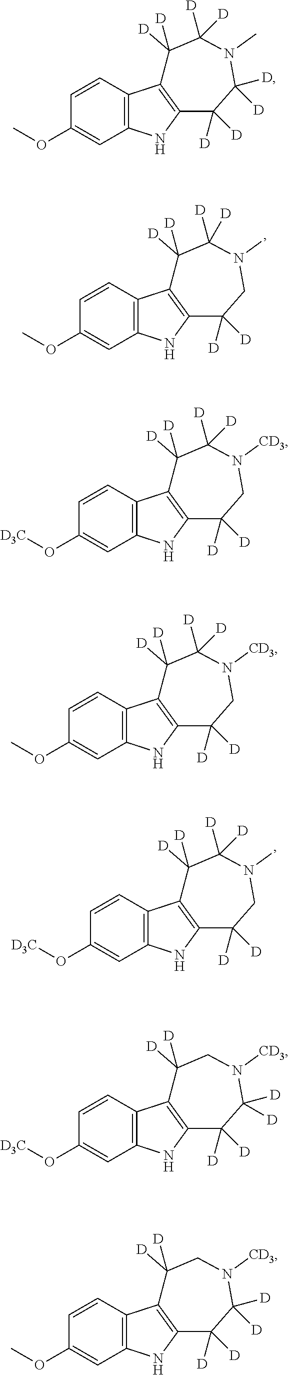

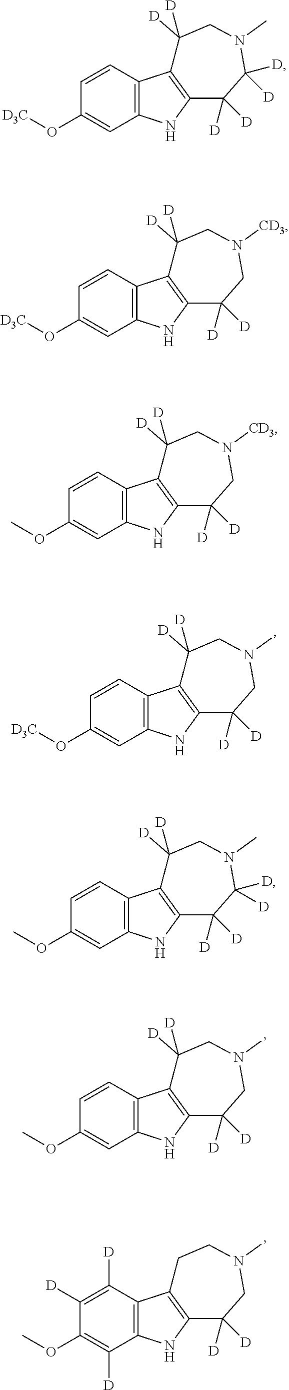

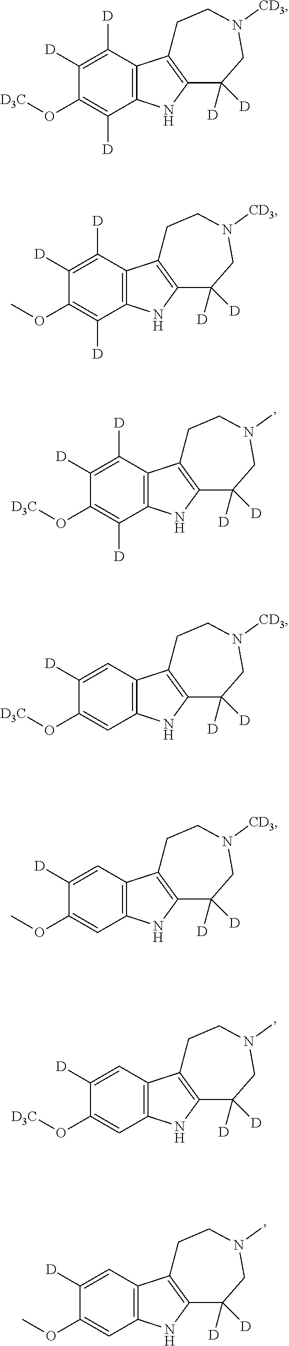

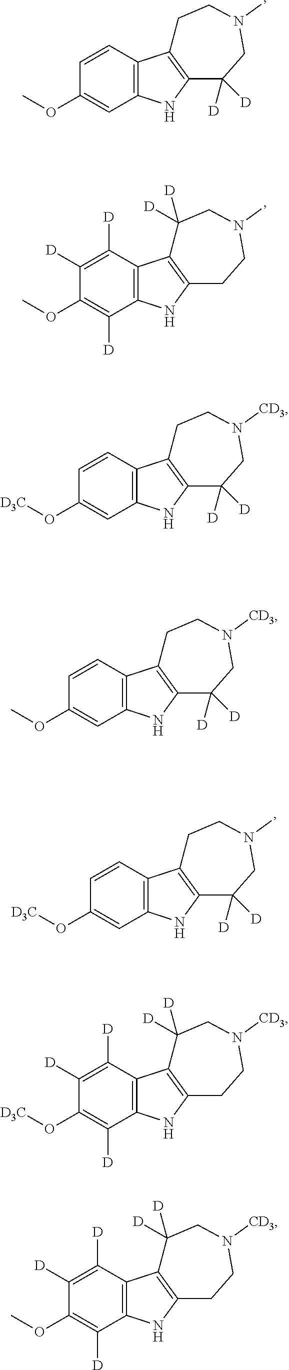

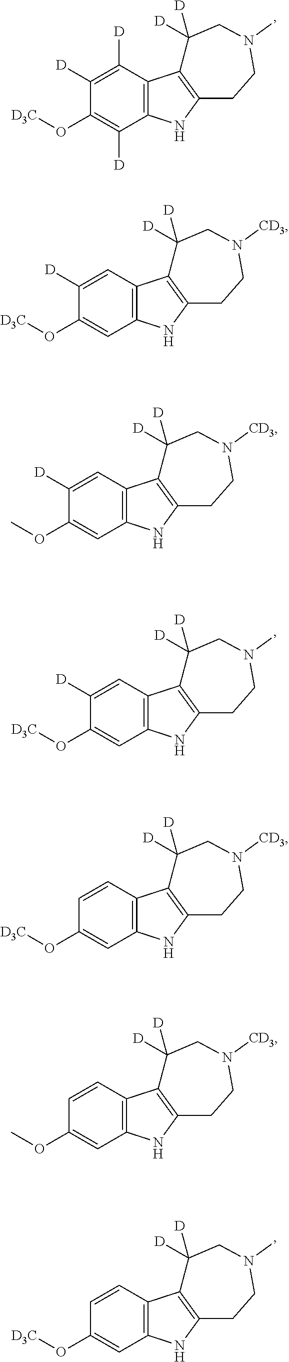

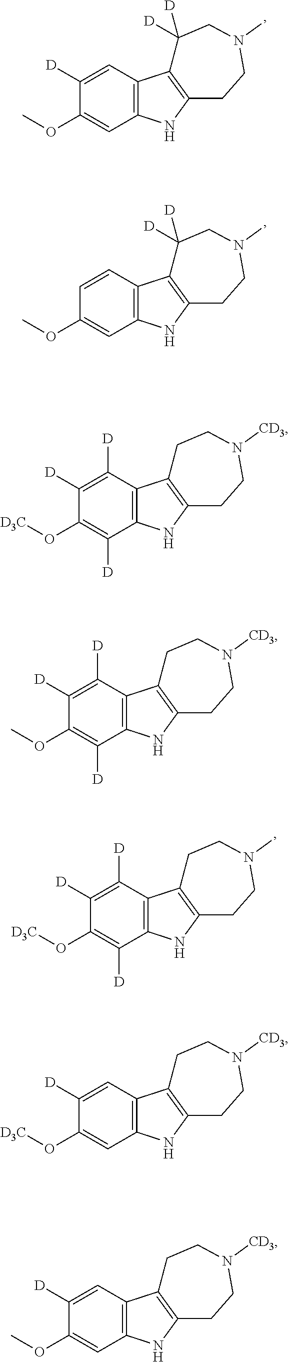

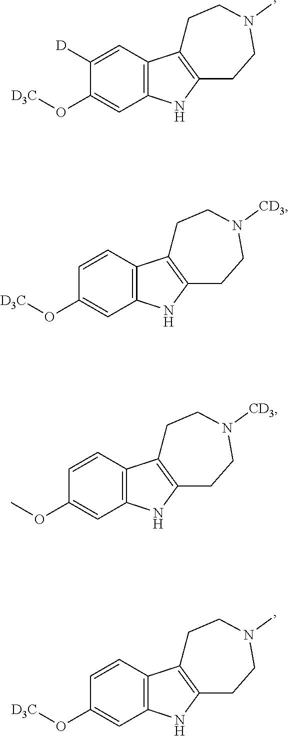

or an enantiomer or a diastereomer thereof, or a pharmaceutically acceptable salt thereof.

### II. Novel Iboga Alkaloid-Like Compounds

Disclosed herein are novel iboga alkaloid-like compounds that have improved properties relative to ibogaine and tabernanthalog, and methods for their use.

In one embodiment of the compounds disclosed herein, the compounds have the structure of Formula II

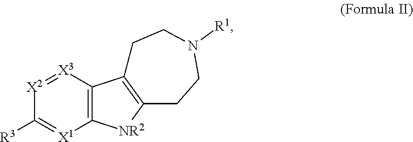

an enantiomer, a diastereomer, an isotopic derivative, or a pharmaceutically acceptable salt thereof, wherein
R¹ and R² independently are selected from hydrogen, deuterium, C₁₋₆ alkyl, —C(O)OR^{a}, —C(O)R^{a}, C₃₋₆ cycloalkyl, and —C(O)NR^{b}R^{b}, wherein alkyl is optionally substituted by one or more —S(O)₂R^{d}, —NR^{b}R^{b}, —OH, or —OD;
X¹ is C(R^{X1}) or N;
X² is C(R^{X2}) or N;
X³ is C(R^{X3}) or N;
R^{X1} is selected from hydrogen, deuterium, and alkyl, or together with R³ forms a heterocyclyl;
R^{X2} is selected from hydrogen, deuterium, halogen, C₁₋₆ alkyl, C₁₋₆ haloalkyl, C₃₋₆ cycloalkyl, —OR^{c} or together with R³ forms a heterocyclyl;
R^{X3} is selected from hydrogen, deuterium, halogen, C₁₋₆ alkyl, C₁₋₆ haloalkyl, C₃₋₆ cycloalkyl, —OR^{c}, —SR^{c}, —SF₅, and —CN;
R³ is selected from halo, C₁₋₆ alkyl, C₁₋₆ haloalkyl, C₃₋₆ cycloalkyl, —OR^{c}, —SR^{c}, —S(O)R^{d}, —S(O)₂R^{d}, —Si(R^{a})₃, and —SF₅, or R³ together with R^{X1} or R^{X2} forms a heterocyclyl;
R^{a} is, for each occurrence, independently selected from the group consisting of C₁₋₆ alkyl, C₁₋₆ haloalkyl, and C₃₋₆ cycloalkyl;
R^{b} is, for each occurrence, independently selected from hydrogen, deuterium, C₁₋₆ alkyl, and C₃₋₆ cycloalkyl, or two R^{b}, together with the nitrogen atom to which they are attached, form a heterocyclylalkyl;
R^{c} is selected from the group consisting of C₁₋₆ alkyl, C₁₋₆ haloalkyl, and C₃₋₆ cycloalkyl; and
R^{d} is selected from the group consisting of C₁₋₆ alkyl, C₁₋₆ haloalkyl, C₃₋₆ cycloalkyl, and —NR^{b}R^{b}; provided that
when R^{X2} is selected from hydrogen, deuterium, and fluoro, R³ is not chloro, cyclopropyl, —S(O)₂CF₃, —SMe, —SEt, —S-iPr, —SCF₃, or —OMe; and
when X² is N, R³ is not —OMe.

In one embodiment of Formula II, disclosed compounds have Formula IIa

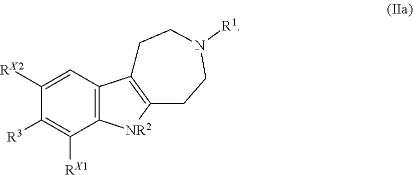

In other embodiments, in compounds disclosed herein according to Formula II at least one of X¹, X² and X³ is N.

In certain embodiments of Formula II, disclosed compounds have a formula selected from Formulas IIb, IIc, IId, IIe, and IIf:

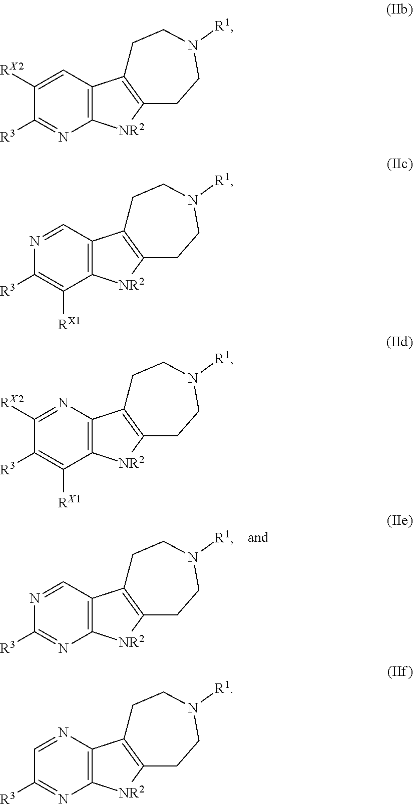

In one embodiment of Formulas II, IIa, IIb, IIc, IId, IIe, and IIf, R³ is C₁₋₆ alkyl, C₁₋₆ haloalkyl, C₃₋₆ cycloalkyl or —OR^{c}. In one embodiment of Formulas II, IIa, IIb, IIc, IId, IIe, and IIf, R³ is C₁₋₆ alkyl, C₃₋₆ cycloalkyl, or —OR^{c}.

In one embodiment of Formulas II, IIa, IIb, IIc, IId, IIe, and IIf, R³ is —OR^{c}.

In one embodiment of Formulas II, IIa, IIb, IIc, IId, IIe, and IIf, R^{c} is C₃₋₆ cycloalkyl.

In one embodiment of Formulas II, IIa, IIb, IIc, IId, IIe, and IIf, R³ is C₃₋₆ cycloalkyl, such as wherein R³ is cyclopropyl.

In some embodiments of Formulas II, IIa, IIb, IIc, IId, IIe, and IIf, R³ is selected from —SR^{c}, —S(O)R^{d}, —S(O)₂R^{d}, —Si(R^{a})₃, and —SF₅.

In one embodiment of Formulas II, IIa, IIb, IIc, IId, IIe, and IIf, R³ is selected from —SR^{c}, —S(O)R^{d}, —S(O)₂R^{d}, —Si(R^{a})₃, and —SF₅, and R¹ and R² are selected from hydrogen, deuterium, and —C(O)R^{a}.

In certain embodiments of Formulas II, IIa, IIb, IIc, IId, IIe, and IIf, R¹ and R² independently are selected from hydrogen, deuterium, C₁₋₆ alkyl, —C(O)OR^{a}, —C(O)R^{a}, C₃₋₆ cycloalkyl, and —C(O)NR^{b}R^{b}, wherein alkyl is optionally substituted by one or more —S(O)₂R^{d}, —NR^{b}R^{b}, —OH, or —OD.

In one embodiment of Formulas II, IIa, IIb, IIc, IId, IIe, and IIf, R¹ and R² are hydrogen, deuterium.

In certain embodiments of Formulas II, IIa, IIb, IIc, IId, IIe, and IIf, R³ is —SR^{c}.

In certain embodiments of Formulas II and IIa, R³ is —SR^{c} and R^{X2} is selected from C₁₋₆ haloalkyl and C₃₋₆ cycloalkyl.

In certain embodiments of Formula II, R³ is —SR^{c} and X² is N.

In certain embodiments of Formulas II, IIa, IIb, IIc, IId, IIe, and IIf, R³ is —SF₅.

In certain embodiments of Formulas II, IIa, IIb, IIc, IId, IIe, and IIf, R³ is selected from —Si(R^{a})₃, and —SF₅, or R³ together with R^{X1} or R^{X2} forms a heterocyclyl.

In certain embodiments of Formulas II and IIa, R³ together with R^{X1} or R^{X2} forms a heterocyclyl, wherein the heterocyclyl is substituted with one or more groups selected from halogen, R^{a}, —C(O)OR^{a} and C(O)R^{d}, wherein R^{a} and R^{d} are as defined for Formula II.

In certain embodiments of Formulas II and IIa, the compounds have the Formula IIg:

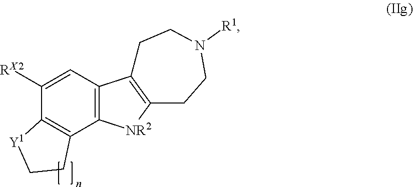

an enantiomer, a diastereomer, an isotopic derivative, or a pharmaceutically acceptable salt thereof, wherein
R¹ and R² independently are selected from hydrogen, deuterium, C₁₋₆ alkyl, —C(O)OR^{a}, —C(O)R^{a}, C₃₋₆ cycloalkyl, and —C(O)NR^{b}R^{b}, wherein alkyl is optionally substituted by one or more —S(O)₂R^{d}, —NR^{b}R^{b}, —OH, or —OD;
R^{X2} is selected from hydrogen, deuterium, halogen, C₁₋₆ alkyl, C₁₋₆ haloalkyl, C₃₋₆ cycloalkyl, or —OR^{c};
Y¹ is O, NH, NR^{a} or NC(O)R^{d};
R^{a} is, for each occurrence, independently selected from the group consisting of C₁₋₆ alkyl, C₁₋₆ haloalkyl, and C₃₋₆ cycloalkyl;
R^{b} is, for each occurrence, independently selected from hydrogen, deuterium, C₁₋₆ alkyl, and C₃₋₆ cycloalkyl, or two R^{b}, together with the nitrogen atom to which they are attached, form a heterocyclylalkyl;
R^{c} is selected from the group consisting of C₁₋₆ alkyl, C₁₋₆ haloalkyl, and C₃₋₆ cycloalkyl;
R^{d} is selected from the group consisting of C₁₋₆ alkyl, C₁₋₆ haloalkyl, C₃₋₆ cycloalkyl, and —NR^{b}R^{b}; and
n is 1 or 2.

In certain embodiments of Formulas II, IIa and IIg, R^{X2} is selected from halogen, C₁₋₆ alkyl, C₁₋₆ haloalkyl and C₃₋₆ cycloalkyl.

In certain embodiments of Formulas II and IIa, R³ and R^{X2} each are independently selected from halogen.

In some embodiments of Formula II, X² is not N, CH or CF.

In some embodiments of Formula II, R³ is not halo, —OR^{c}, —SR^{c}, or —S(O)₂R^{d}.

In some embodiments of Formula II, the compound is not

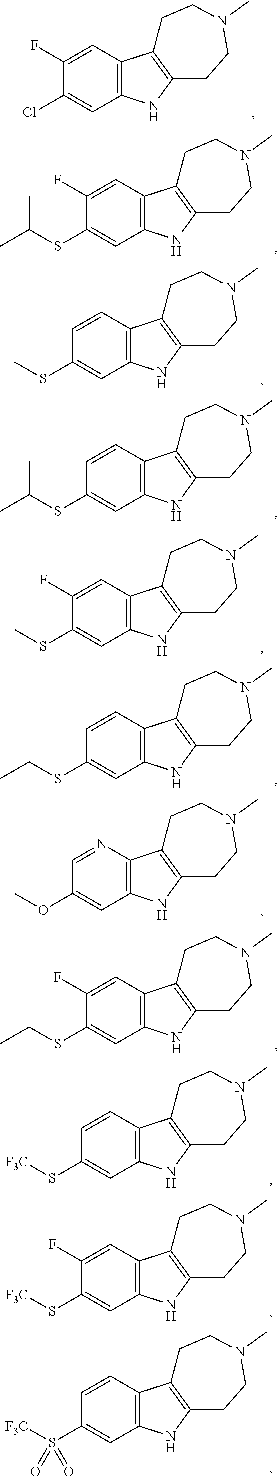

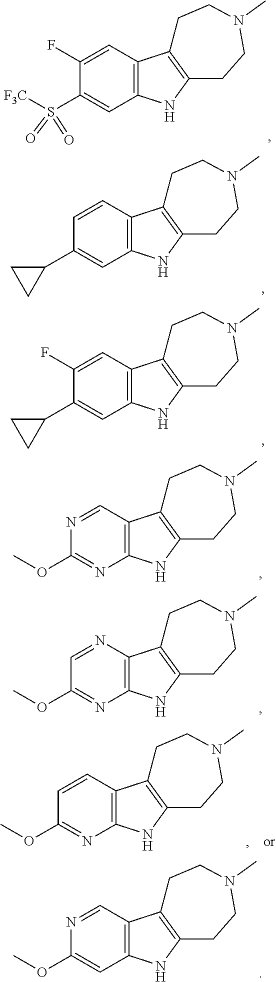

In certain embodiments of Formulas II, IIa, IIb, IIc, IId, IIe, and/or IIf, disclosed compounds include:

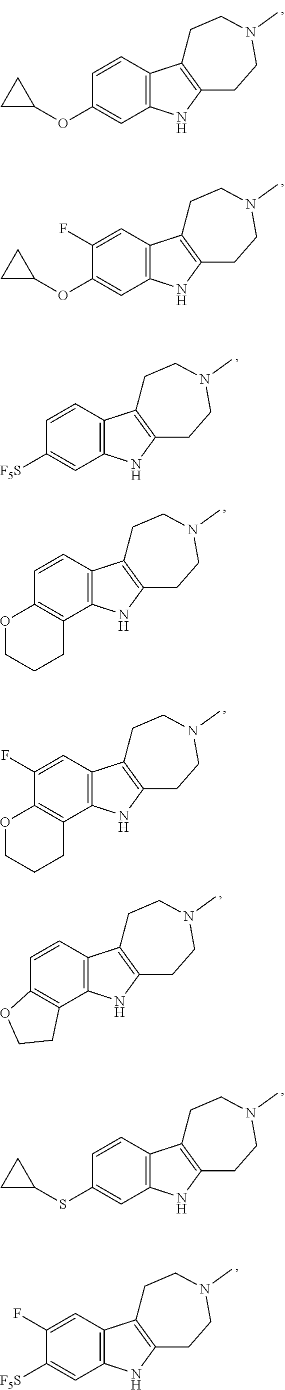

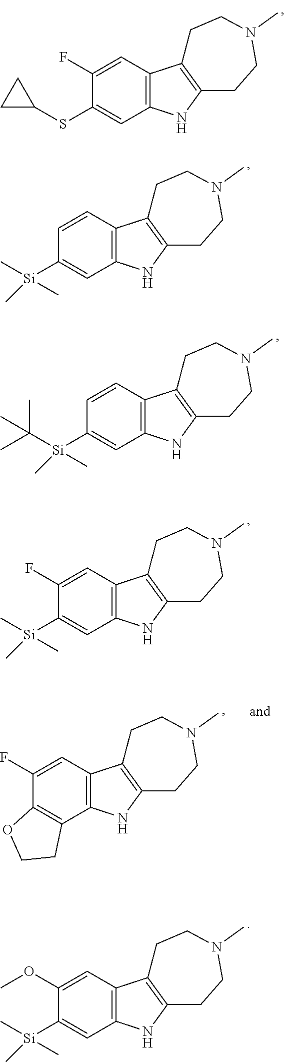

The compounds of the present invention can also be in salt forms, such as acid or base salts of the compounds of the present invention. Illustrative examples of pharmaceutically acceptable acid salts are mineral acid (hydrochloric acid, hydrobromic acid, phosphoric acid, and the like) salts, organic acid (fumaric acid, acetic acid, propionic acid, glutamic acid, citric acid, tartaric acid and the like) salts. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

The present invention includes all tautomers and stereoisomers of compounds of Formulas I, I-1, I-1A I-1B, II, IIa, IIb, IIc, IId, IIe, IIf, and IIg either in admixture or in pure or substantially pure form. The compounds of the present invention can have asymmetric centers at the carbon atoms, and therefore the compounds of the present invention can exist in diastereomeric or enantiomeric forms or mixtures thereof. All conformational isomers (e.g., cis and trans isomers) and all optical isomers (e.g., enantiomers and diastereomers), racemic, diastereomeric and other mixtures of such isomers.

In addition, all physical forms of the compounds of Formulas I, I-1, I-1A I-1B, II, IIa, IIb, IIc, IId, IIe, IIf, IIg, Table 1, are intended herein, including the compounds of Formulas I, I-1, I-1A I-1B, II, IIa, IIb, IIc, IId, IIe, IIf, IIg, Table 1, in the form of solvates, such as hydrates. Moreover, non-crystalline and crystalline forms of the compounds of Formulas I, I-1, I-1A I-1B, II, IIa, IIb, IIc, IId, IIe, IIf, IIg, Table 1, including amorphous forms, isomorphs and polymorphs are within the scope of the present invention.

Exemplary compounds according to the present invention are chiral. Such compounds can be prepared as is known to those of skill in the art can be prepared using in diastereomeric, enantiomeric or racemic mixtures as contemplated herein. Furthermore, diastereomer and enantiomer products can be separated by chromatography, fractional crystallization or other methods known to those of skill in the art.

TABLE 1
Representative compounds include the following.

| Compound | Structure |
|---|---|
| Number | SMILES |
| 1 | 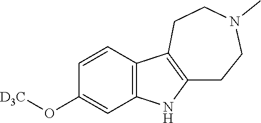 [SMILES: [2H]C([2H])([2H])Oc1ccc2c3c([nH]c2c1)CCN(C)CC3] |
| | CN(CC1)CCC2═C1C3═C(N2) |
| | C═C(OC([2H])([2H])[2H])C═C3 |
| 2 | 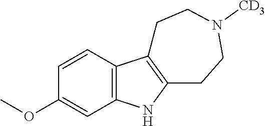 [SMILES: [2H]C([2H])([2H])N1CCc2[nH]c3cc(OC)ccc3c2CC1] |
| | COC1═CC(NC2═C3CCN(C([2H]) |
| | ([2H])[2H])CC2)═C3C═C1 |
| 3 | 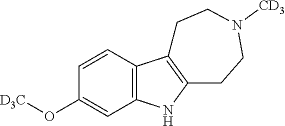 [SMILES: [2H]C([2H])([2H])Oc1ccc2c3c([nH]c2c1)CCN(C([2H])([2H])[2H])CC3] |
| | [2H]C([2H])([2H])OC1═CC |
| | (NC2═C3CCN(C([2H])([2H]) |
| | [2H])CC2)═C3C═C1 |
| 4 | 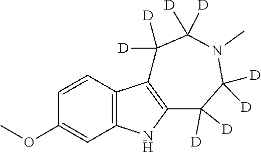 [SMILES: [2H]C1([2H])c2[nH]c3cc(OC)ccc3c2C([2H])([2H])C([2H])([2H])N(C)C1([2H])[2H]] |
| | COC1═CC(NC2═C3C([2H]) |
| | ([2H])C([2H])([2H])N(C)C([2H]) |
| | ([2H])C2([2H])[2H])═C3C═C1 |
| 5 | 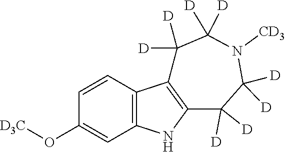 [SMILES: [2H]C([2H])([2H])Oc1ccc2c3c([nH]c2c1)C([2H])([2H])C([2H])([2H])N(C([2H])([2H])[2H])C([2H])([2H])C3([2H])[2H]] |
| | [2H]C1([2H))C([2H[2H]) |
| | N(C([2H])([2H])[2H])C([2H])([2H]) |
| | C([2H])([2H])C2═C1C3═C(N2) |
| | C═C(OC([2HD[2HD)[2H))C═C3 |
| 6 | 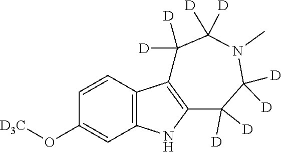 [SMILES: [2H]C([2H])([2H])Oc1ccc2c3c([nH]c2c1)C([2H])([2H])C([2H])([2H])N(C)C([2H])([2H])C3([2H])[2H]] |
| | CN(C([2H])([2H])C1([2H])[2H]) |
| | C([2H])([2H])C([2H])([2H]) |
| | C2═C1C3═C(N2)C═C(OC([2H]) |
| | ([2H])[2H])C═C3 |
| 7 | 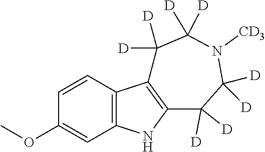 [SMILES: [2H]C([2H])([2H])N1C([2H])([2H])C([2H])([2H])c2[nH]c3cc(OC)ccc3c2C([2H])([2H])C1([2H])[2H]] |
| | COC1═CC(NC2═C3C([2H])([2H]) |
| | C([2H])([2H])N(C([2H])([2H]) |
| | [2H])C([2H])([2H])C2([2H]) |
| | [2H])═C3C═C1 |
| 8 | 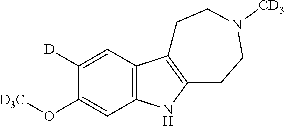 [SMILES: [2H]c1cc2c3c([nH]c2cc1OC([2H])([2H])[2H])CCN(C([2H])([2H])[2H])CC3] |
| | [2H]C1═CC2═C(NC3═C2CCN |
| | (C([2H])([2H])[2H])CC3)C═C1OC |
| | ([2H])([2H])[2H] |
| 9 | 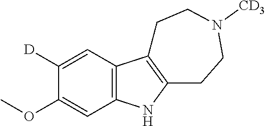 [SMILES: [2H]c1cc2c3c([nH]c2cc1OC)CCN(C([2H])([2H])[2H])CC3] |
| | COC1═CC(NC2═C3CCN(C([2H]) |
| | ([2H])[2H])CC2)═C3C═C1[2H] |
| 10 | 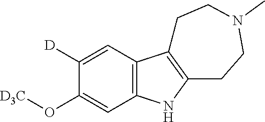 [SMILES: [2H]c1cc2c3c([nH]c2cc1OC([2H])([2H])[2H])CCN(C)CC3] |
| | CN(CC1)CCC2═C1C3═C(N2) |
| | C═C(OC([2H])([2H])[2H])C([2H])═C3 |
| 11 | 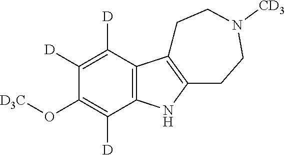 [SMILES: [2H]c1c(OC([2H])([2H])[2H])c([2H])c2[nH]c3c(c2c1[2H])CCN(C([2H])([2H])[2H])CC3] |
| | [2H]C1═C([2H])C2═C(NC3═C2CCN |
| | (C([2H])([2H])[2H])CC3) |
| | C([2H])═C1OC([2H])([2H])[2H] |
| 12 | 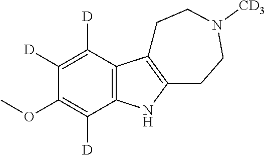 [SMILES: [2H]c1c(OC)c([2H])c2[nH]c3c(c2c1[2H])CCN(C([2H])([2H])[2H])CC3] |
| | COC1═C([2H])C(NC2═C3CCN |
| | (C([2H])([2H])[2H])CC2)═C3C([2H])═C1[2H] |
| 13 | 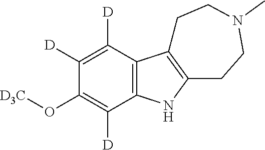 [SMILES: [2H]c1c(OC([2H])([2H])[2H])c([2H])c2[nH]c3c(c2c1[2H])CCN(C)CC3] |
| | CN(CC1)CCC2═C1C3═C(N2)C |
| | ([2H])═C(OC([2H])([2H])[2H])C([2H)═C3[2H] |
| 14 | 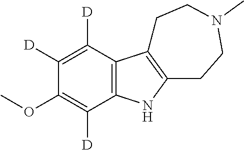 |
| | COC1═C([2H])C(NC2═C3CCN |
| | (C)CC2)═C3C([2H])═C1[2H] |
| 15 | 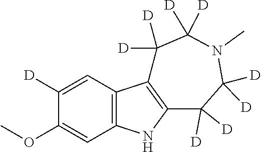 [SMILES: [2H]c1cc2c3c([nH]c2cc1OC)C([2H])([2H])C([2H])([2H])N(C)C([2H])([2H])C3([2H])[2H]] |
| | COC1═CC(NC2═C3C([2H])([2H]) |
| | C([2H])([2H])N(C)C([2H])([2H]) |
| | C2([2H])[2H])═C3C═C1[2H] |
| 16 | 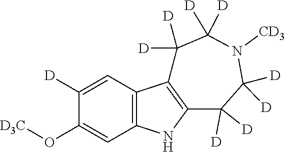 [SMILES: [2H]c1cc2c3c([nH]c2cc1OC([2H])([2H])[2H])C([2H])([2H])C([2H])([2H])N(C([2H])([2H])[2H])C([2H])([2H])C3([2H])[2H]] |
| | [2H]C1([2H])C([2H])([2H]) |
| | N(C([2H])([2H])[2H])C([2H])([2H]) |
| | C([2H])([2H])C2═C1C3═C(N2) |
| | C═C(OC([2H])([2H])[2H])C([2H])═C3 |
| 17 | 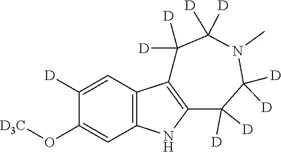 [SMILES: [2H]c1cc2c3c([nH]c2cc1OC([2H])([2H])[2H])C([2H])([2H])C([2H])([2H])N(C)C([2H])([2H])C3([2H])[2H]] |
| | CN(C([2H])([2H])C1([2H])[2H]) |
| | C([2H])([2H])C([2H])([2H]) |
| | C2═C1C3═C(N2)C═C(OC([2H]) |
| | ([2H])[2H])C([2H])═C3 |
| 18 | 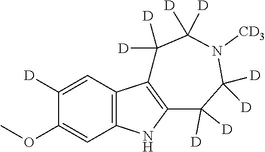 [SMILES: [2H]c1cc2c3c([nH]c2cc1OC)C([2H])([2H])C([2H])([2H])N(C([2H])([2H])[2H])C([2H])([2H])C3([2H])[2H]] |
| | COC1═CC(NC2═C3C([2H])([2H]) |
| | C([2H])([2H])N(C([2H])([2H]) |
| | [2H])C([2H])([2H])C2([2H]) |
| | [2H])═C3C═C1[2H] |
| 19 | 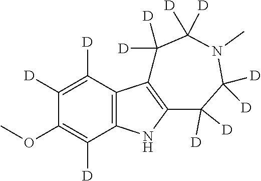 [SMILES: [2H]c1c(OC)c([2H])c2[nH]c3c(c2c1[2H])C([2H])([2H])C([2H])([2H])N(C)C([2H])([2H])C3([2H])[2H]] |
| | COC1═C([2H])C(NC2═C3C([2H]) |
| | ([2H])C([2H])([2H])N(C)C([2H])([2H]) |
| | C2([2H])[2H])═C3C([2H])═C1[2H] |
| 20 | 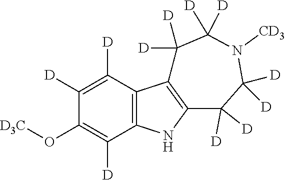 [SMILES: [2H]c1c(OC([2H])([2H])[2H])c([2H])c2[nH]c3c(c2c1[2H])C([2H])([2H])C([2H])([2H])N(C([2H])([2H])[2H])C([2H])([2H])C3([2H])[2H]] |
| | [2H]C1([2H])C([2H][2H])N |
| | (C(2H])([2H])[2H])C([2H])([2H]) |
| | C([2H])([2H])C2═C1C3═C(N2) |
| | C([2H])═C(OC([2H])([2H])[2H]) |
| | C([2H])═C3[2H] |
| 21 | 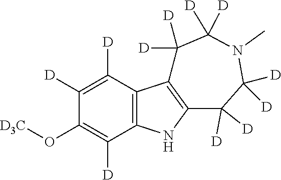 [SMILES: [2H]c1c(OC([2H])([2H])[2H])c([2H])c2[nH]c3c(c2c1[2H])C([2H])([2H])C([2H])([2H])N(C)C([2H])([2H])C3([2H])[2H]] |
| | CN(C([2H])([2H])C1([2H])[2H]) |
| | C([2H])([2H])C([2H])([2H]) |
| | C2═C1C3═C(N2)C([2H])═C(OC |
| | ([2H])([2H][2H)C([2H))═C3[2H] |
| 22 | 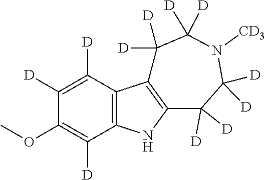 [SMILES: [2H]c1c(OC)c([2H])c2[nH]c3c(c2c1[2H])C([2H])([2H])C([2H])([2H])N(C([2H])([2H])[2H])C([2H])([2H])C3([2H])[2H]] |
| | COC1═C([2H])C(NC2═C3C([2H]) |
| | ([2H])C([2H])([2H])N(C([2H]) |
| | ([2H])[2H])C([2H])([2H])C2([2H]) |
| | [2H])═C3C([2H])═C1[2H] |
| 23 | 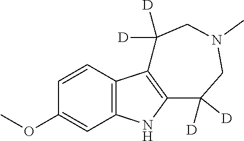 |
| | COC1═CC(NC2═C3C([2H])([2H]) |
| | CN(C)CC2([2H][2H))═C3C═C1 |
| 24 | 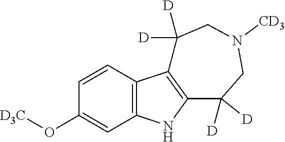 [SMILES: [2H]C([2H])([2H])Oc1ccc2c3c([nH]c2c1)C([2H])([2H])CN(C([2H])([2H])[2H])CC3([2H])[2H]] |
| | [2H]C1([2H])CN(C([2H])([2H])[2H]) |
| | CC([2H])([2H])C2═C1C3═C |
| | (N2)C═C(OC([2H])([2H])[2H])C═C3 |
| 25 | 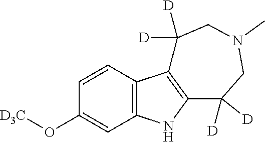 [SMILES: [2H]C([2H])([2H])Oc1ccc2c3c([nH]c2c1)C([2H])([2H])CN(C)CC3([2H])[2H]] |
| | CN(CC1([2H])[2H])CC([2H])([2H]) |
| | C2═C1C3═C(N2)C═C(OC([2H]) |
| | ([2H])[2H])C═C3 |
| 26 | 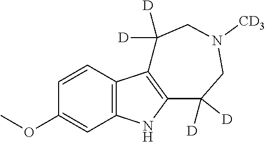 [SMILES: [2H]C1([2H])CN(C([2H])([2H])[2H])CC([2H])([2H])c2c1[nH]c1cc(OC)ccc21] |
| | COC1═CC(NC2═C3C([2H])([2H]) |
| | CN(C([2H])([2H])[2H])CC2([2H]) |
| | [2H])═C3C═C1 |
| 27 | 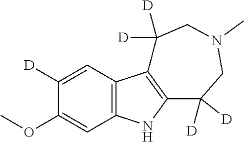 |
| | COC1═CC(NC2═C3C([2H])([2H]) |
| | CN(C)CC2([2H])[2H])═C3C═C1[2H] |
| 28 | 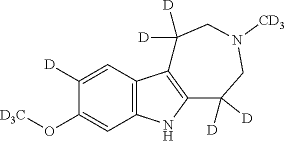 [SMILES: [2H]c1cc2c3c([nH]c2cc1OC([2H])([2H])[2H])C([2H])([2H])CN(C([2H])([2H])[2H])CC3([2H])[2H]] |
| | [2H]C1([2H])CN(C([2H])([2H])[2H]) |
| | CC([2H])([2H])C2═C1C3═C(N2) |
| | C═C(OC([2H])([2H])[2H])C([2H])═C3 |
| 29 | 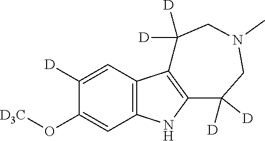 [SMILES: [2H]c1cc2c3c([nH]c2cc1OC([2H])([2H])[2H])C([2H])([2H])CN(C)CC3([2H])[2H]] |
| | CN(CC1([2H])[2H])CC([2H])([2H]) |
| | C2═C1C3═C(N2)C═C(OC([2H]) |
| | ([2H])[2H])C([2H])═C3 |
| 30 | 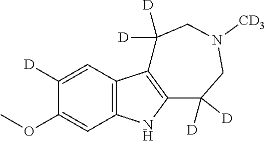 [SMILES: [2H]c1cc2c3c([nH]c2cc1OC)C([2H])([2H])CN(C([2H])([2H])[2H])CC3([2H])[2H]] |
| | COC1═CC(NC2═C3C([2H])([2H]) |
| | CN(C([2H])([2H])[2H])CC2([2H]) |
| | [2H])═C3C═C1[2H] |
| 31 | 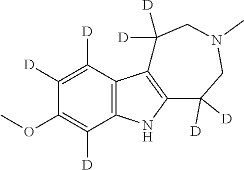 |
| | COC1═C([2H])C(NC2═C3C([2H]) |
| | ([2H])CN(C)CC2([2H])[2H])═C3C([2H])═C1[2H] |
| 32 | 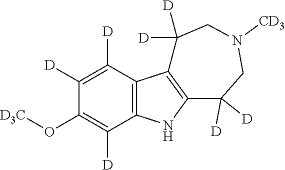 [SMILES: [2H]c1c(OC([2H])([2H])[2H])c([2H])c2[nH]c3c(c2c1[2H])C([2H])([2H])CN(C([2H])([2H])[2H])CC3([2H])[2H]] |
| | [2H]C1([2H])CN(C([2H])([2H]) |
| | [2H])CC([2H])([2H])C2═C1C3═C |
| | (N2)C([2H])═C(OC([2H])([2H]) |
| | [2H])C([2H])═C3[2H] |
| 33 | 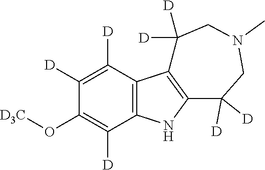 [SMILES: [2H]c1c(OC([2H])([2H])[2H])c([2H])c2[nH]c3c(c2c1[2H])C([2H])([2H])CN(C)CC3([2H])[2H]] |
| | CN(CC1([2H])[2H])CC([2H])([2H]) |
| | C2═C1C3═C(N2)C([2H])═C(OC |
| | ([2H])([2H])[2H])C([2H])═C3[2H] |
| 34 | 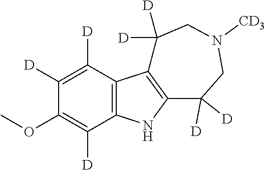 [SMILES: [2H]c1c(OC)c([2H])c2[nH]c3c(c2c1[2H])C([2H])([2H])CN(C([2H])([2H])[2H])CC3([2H])[2H]] |
| | COC1═C([2H])C(NC2═C3C([2H]) |
| | ([2H])CN(C([2H])([2H])[2H])CC2 |
| | ([2H])[2H])═C3C([2H])═C1[2H] |
| 35 | 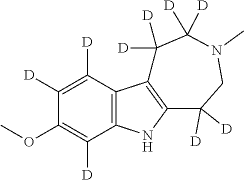 |
| | COC1═C([2H])C(NC2═C3C([2H]) |
| | ([2H])C([2H])([2H])N(C)CC2 |
| | ([2H])[2H])═C3C([2H])═C1[2H] |
| 36 | 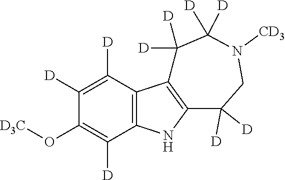 [SMILES: [2H]c1c(OC([2H])([2H])[2H])c([2H])c2[nH]c3c(c2c1[2H])C([2H])([2H])C([2H])([2H])N(C([2H])([2H])[2H])CC3([2H])[2H]] |
| | [2H]C1([2H])C([2H])([2H])N(C |
| | ([2H])([2H])[2H])CC([2H])([2H]) |
| | C2═C1C3═C(N2)C([2H])═C(OC |
| | ([2H])([2H])[2H])C([2H])═C3[2H] |
| 37 | 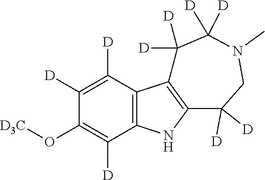 [SMILES: [2H]c1c(OC([2H])([2H])[2H])c([2H])c2[nH]c3c(c2c1[2H])C([2H])([2H])C([2H])([2H])N(C)CC3([2H])[2H]] |
| | CN(C([2H])([2H])C1([2H])[2H]) |
| | CC([2H])([2H])C2═C1C3═C(N2)C |
| | ([2H])═C(OC([2H])([2H])[2H])C([2H])═C3[2H] |
| 38 | 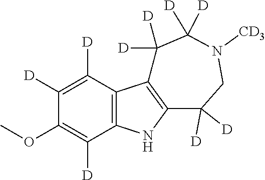 [SMILES: [2H]c1c(OC)c([2H])c2[nH]c3c(c2c1[2H])C([2H])([2H])C([2H])([2H])N(C([2H])([2H])[2H])CC3([2H])[2H]] |
| | COC1═C([2H])C(NC2═C3C([2H]) |
| | ([2H])C([2H])([2H])N(C([2H]) |
| | ([2H])[2H])CC2([2H])[2H])═C3C([2H])═C1[2H] |
| 39 | 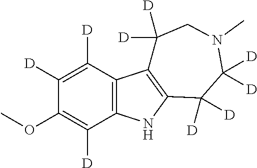 [SMILES: [2H]c1c(OC)c([2H])c2[nH]c3c(c2c1[2H])C([2H])([2H])CN(C)C([2H])([2H])C3([2H])[2H]] |
| | COC1═C([2H])C(NC2═C3C([2H]) |
| | ([2H])CN(C)C([2H])([2H])C2 |
| | ([2H])[2H])═C3C([2H])═C1[2H] |
| 40 | 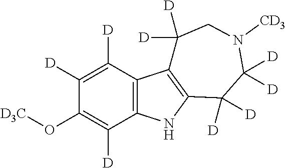 [SMILES: [2H]c1c(OC([2H])([2H])[2H])c([2H])c2[nH]c3c(c2c1[2H])C([2H])([2H])CN(C([2H])([2H])[2H])C([2H])([2H])C3([2H])[2H]] |
| | [2H]C1([2H])CN(C([2H])([2H]) |
| | [2H])C([2H])([2H])C([2H])([2H]) |
| | C2═C1C3═C(N2)C([2H])═C(OC |
| | ([2H])([2H])[2H])C([2H])═C3[2H] |
| 41 | 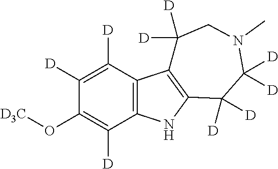 [SMILES: [2H]c1c(OC([2H])([2H])[2H])c([2H])c2[nH]c3c(c2c1[2H])C([2H])([2H])CN(C)C([2H])([2H])C3([2H])[2H]] |
| | CN(CC1([2H])[2H])C([2H])([2H]) |
| | C([2H])([2H])C2═C1C3═C(N2)C |
| | ([2H])═C(OC([2H])([2H])[2H])C |
| | ([2H])═C3[2H] |
| 42 | 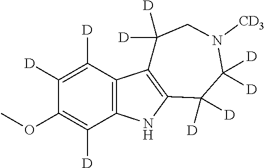 [SMILES: [2H]c1c(OC)c([2H])c2[nH]c3c(c2c1[2H])C([2H])([2H])CN(C([2H])([2H])[2H])C([2H])([2H])C3([2H])[2H]] |
| | COC1═C([2H])C(NC2═C3C([2H]) |
| | ([2H])CN(C([2H])([2H])[2H])C |
| | ([2H])([2H])C2([2H])[2H])═C3C |
| | ([2H])═C1[2H] |
| 43 | 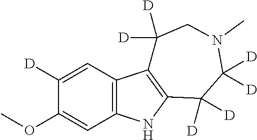 [SMILES: [2H]c1cc2c3c([nH]c2cc1OC)C([2H])([2H])C([2H])([2H])N(C)CC3([2H])[2H]] |
| | COC1═CC(NC2═C3C([2H])([2H]) |
| | CN(C)C([2H])([2H])C2([2H]) |
| | [2H])═C3C═C1[2H] |
| 44 | 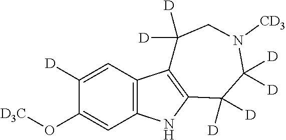 [SMILES: [2H]c1cc2c3c([nH]c2cc1OC([2H])([2H])[2H])C([2H])([2H])C([2H])([2H])N(C([2H])([2H])[2H])CC3([2H])[2H]] |
| | [2H]C1([2H])CN(C([2H])([2H]) |
| | [2H])C([2H])([2H])C([2H])([2H]) |
| | C2═C1C3═C(N2)C═C(OC([2H]) |
| | ([2H])[2H)C([2H])═C3 |
| 45 | 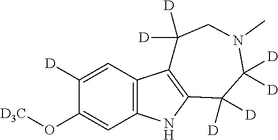 [SMILES: [2H]c1cc2c3c([nH]c2cc1OC([2H])([2H])[2H])C([2H])([2H])C([2H])([2H])N(C)CC3([2H])[2H]] |
| | CN(CC1([2H])[2H])C([2H])([2H]) |
| | C([2H])([2H])C2═C1C3═C(N2) |
| | C═C(OC([2H])([2H])[2H]) |
| | C([2H])═C3 |
| 46 | 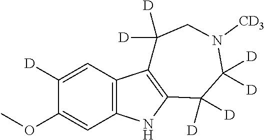 [SMILES: [2H]c1cc2c3c([nH]c2cc1OC)C([2H])([2H])C([2H])([2H])N(C([2H])([2H])[2H])CC3([2H])[2H]] |
| | COC1═CC(NC2═C3C([2H])([2H]) |
| | CN(C([2H])([2H])[2H])C([2H]) |
| | ([2H])C2([2H])[2H])═C3C═C1[2H] |
| 47 | 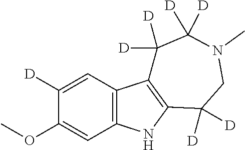 |
| | COC1═CC(NC2═C3C([2H])([2H]) |
| | C([2H])([2H])N(C)CC2([2H]) |
| | [2H])═C3C═C1[2H] |
| 48 | 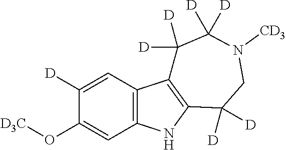 [SMILES: [2H]c1cc2c3c([nH]c2cc1OC([2H])([2H])[2H])C([2H])([2H])CN(C([2H])([2H])[2H])C([2H])([2H])C3([2H])[2H]] |
| | [2H]C1([2H))C([2H)[2H))N(C |
| | ([2H])([2H])[2H])CC([2H])([2H]) |
| | C2═C1C3═C(N2)C═C(OC([2H]) |
| | ([2H])[2H])C([2H])═C3 |
| 49 | 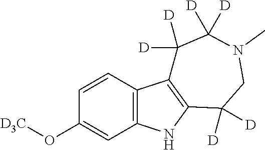 [SMILES: [2H]C([2H])([2H])Oc1ccc2c3c([nH]c2c1)C([2H])([2H])CN(C)C([2H])([2H])C3([2H])[2H]] |
| | CN(C([2H])([2H])C1([2H])[2H]) |
| | CC([2H])([2H])C2═C1C3═C(N2) |
| | C═C(OC([2H][2H])[2H])C([2H])═C3 |
| 50 | 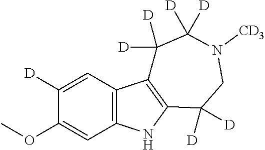 [SMILES: [2H]c1cc2c3c([nH]c2cc1OC)C([2H])([2H])CN(C([2H])([2H])[2H])C([2H])([2H])C3([2H])[2H]] |
| | COC1═CC(NC2═C3C([2H])([2H]) |
| | C([2H])([2H])N(C([2H])([2H]) |
| | [2H])CC2([2H])[2H])═C3C═C1[2H] |
| 51 | 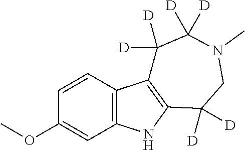 |
| | COC1═CC(NC2═C3C([2H])([2H]) |
| | C([2H])([2H])N(C([2H])([2H]) |
| | [2H])CC2([2H])[2H])═C3C═C1[2H] |
| 52 | 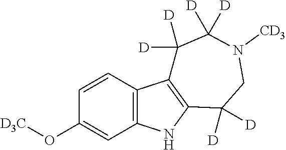 [SMILES: [2H]C([2H])([2H])Oc1ccc2c3c([nH]c2c1)C([2H])([2H])CN(C([2H])([2H])[2H])C([2H])([2H])C3([2H])[2H]] |
| | [2H]C1([2H])C([2H])([2H])N(C |
| | ([2H])([2H])[2H])CC([2H])([2H]) |
| | C2═C1C3═C(N2)C═C(OC([2H]) |
| | ([2H])[2H)C═C3 |
| 53 | 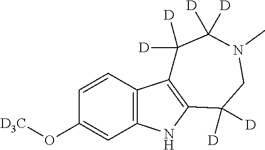 [SMILES: [2H]C([2H])([2H])Oc1ccc2c3c([nH]c2c1)C([2H])([2H])CN(C)C([2H])([2H])C3([2H])[2H]] |
| | CN(C([2H])([2H])C1([2H])[2H]) |
| | CC([2H])([2H])C2═C1C3═C(N2) |
| | C═C(OC([2H])([2H])[2H])C═C3 |
| 54 | 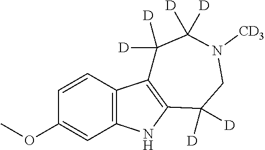 [SMILES: [2H]C1([2H])CN(C([2H])([2H])[2H])C([2H])([2H])C([2H])([2H])c2c1[nH]c1cc(OC)ccc21] |
| | COC1═CC(NC2═C3C([2H])([2H]) |
| | C([2H])([2H])N(C([2H])([2H]) |
| | [2H])CC2([2H])[2H])═C3C═C1 |
| 55 | 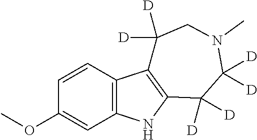 [SMILES: [2H]C1([2H])CN(C)C([2H])([2H])C([2H])([2H])c2[nH]c3cc(OC)ccc3c21] |
| | COC1═CC(NC2═C3C([2H])([2H]) |
| | CN(C)C([2H])([2H])C2([2H]) |
| | [2H])═C3C═C1 |
| 56 | 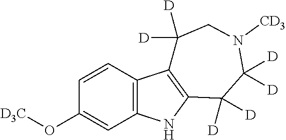 [SMILES: [2H]C([2H])([2H])Oc1ccc2c3c([nH]c2c1)C([2H])([2H])C([2H])([2H])N(C([2H])([2H])[2H])CC3([2H])[2H]] |
| | [2H]C1([2H])CN(C([2H])([2H]) |
| | [2H])C([2H])([2H])C([2H])([2H]) |
| | C2═C1C3═C(N2)C═C(OC([2H]) |
| | ([2H])[2H])C═C3 |
| 57 | 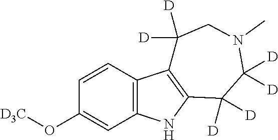 [SMILES: [2H]C([2H])([2H])Oc1ccc2c3c([nH]c2c1)C([2H])([2H])C([2H])([2H])N(C)CC3([2H])[2H]] |
| | CN(CC1([2H])[2H])C([2H])([2H]) |
| | C([2H])([2H])C2═C1C3═C(N2) |
| | C═C(OC([2H])([2H])[2H])C═C3 |
| 58 | 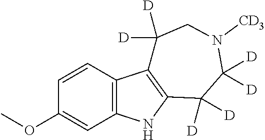 [SMILES: [2H]C1([2H])CN(C([2H])([2H])[2H])C([2H])([2H])C([2H])([2H])c2[nH]c3cc(OC)ccc3c21] |
| | COC1═CC(NC2═C3C([2H])([2H)] |
| | CN(C([2H])([2H])[2H])C([2H]) |
| | ([2H])C2([2H])[2H])═C3C═C1 |
| 59 | 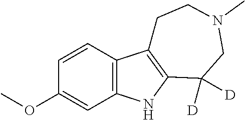 |
| | COC1═CC(NC2═C3CCN(C) |
| | CC2([2H])[2H)═C3C═Cl |
| 60 | 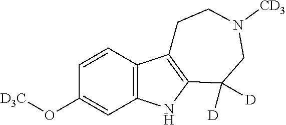 [SMILES: [2H]C([2H])([2H])Oc1ccc2c3c([nH]c2c1)C([2H])([2H])CN(C([2H])([2H])[2H])CC3] |
| | [2H]C(CN(C([2H])([2H])[2H])CC1) |
| | ([2H])C2═C1C3═C(N2)C═C(OC |
| | ([2H])([2H])[2H])C═C3 |
| 61 | 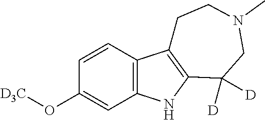 [SMILES: [2H]C([2H])([2H])Oc1ccc2c3c([nH]c2c1)C([2H])([2H])CN(C)CC3] |
| | CN(CC1)CC([2H])([2H]) |
| | C2═C1C3═C(N2)C═C(OC([2H]) |
| | ([2H])[2H])C═C3 |
| 62 | 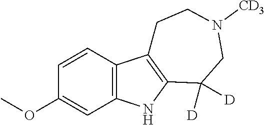 [SMILES: [2H]C1([2H])CN(C([2H])([2H])[2H])CCc2c1[nH]c1cc(OC)ccc21] |
| | COC1═CC(NC2═C3CCN(C([2H]) |
| | ([2H])[2H])CC2([2H])[2H])═C3C═C1 |
| 63 | 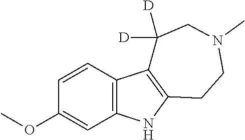 |
| | COC1═CC(NC2═C3C([2H])([2H]) |
| | CN(C)CC2)═C3C═C1 |
| 64 | 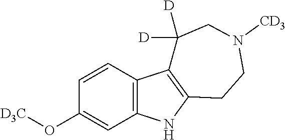 [SMILES: [2H]C([2H])([2H])Oc1ccc2c3c([nH]c2c1)CCN(C([2H])([2H])[2H])CC3([2H])[2H]] |
| | [2H]C1([2H])CN(C([2H])([2H]) |
| | [2H])CCC2═C1C3═C(N2)C═C(OC |
| | ([2H])([2H])[2H])C═C3 |
| 65 | 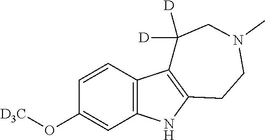 [SMILES: [2H]C([2H])([2H])Oc1ccc2c3c([nH]c2c1)CCN(C)CC3([2H])[2H]] |
| | CN(CC1([2H])[2H])CCC2═C1C3═C |
| | (N2)C═C(OC([2H])([2H])[2H])C═C3 |
| 66 | 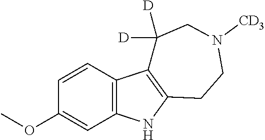 [SMILES: [2H]C1([2H])CN(C([2H])([2H])[2H])CCc2[nH]c3cc(OC)ccc3c21] |
| | COC1═CC(NC2═C3C([2H])([2H]) |
| | CN(C([2H])([2H])[2H])CC2)═C3C═C1 |
| 67 | 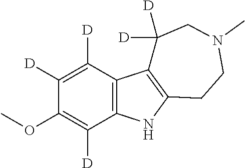 |
| | COC1═C([2H])C(NC2═C3C([2H]) |
| | ([2H])CN(C)CC2)═C3C([2H])═C1[2H] |
| 68 | 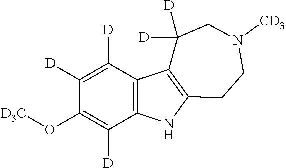 [SMILES: [2H]c1c(OC([2H])([2H])[2H])c([2H])c2[nH]c3c(c2c1[2H])C([2H])([2H])CN(C([2H])([2H])[2H])CC3] |
| | [2H]C1([2H])CN(C([2H])([2H]) |
| | [2H])CCC2═C1C3═C(N2)C([2H])═C(OC |
| | ([2H])([2H])[2H])C([2H])═C3[2H] |
| 69 | 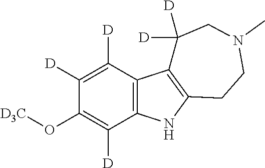 [SMILES: [2H]c1c(OC([2H])([2H])[2H])c([2H])c2[nH]c3c(c2c1[2H])C([2H])([2H])CN(C)CC3] |
| | CN(CC1([2H])[2H]) |
| | CCC2═C1C3═C(N2)C([2H])═C(OC |
| | ([2H])([2H])[2H))C([2H))═C3[2H] |
| 70 | 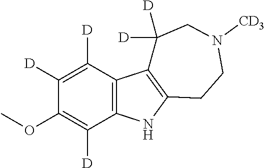 [SMILES: [2H]c1c(OC)c([2H])c2[nH]c3c(c2c1[2H])C([2H])([2H])CN(C([2H])([2H])[2H])CC3] |
| | COC1═C([2H])C(NC2═C3C([2H]) |
| | ([2H])CN(C([2H])([2H])[2H]) |
| | CC2)═C3C([2H])═C1[2H] |
| 71 | 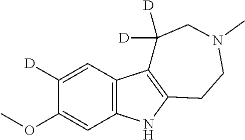 |
| | COC1═CC(NC2═C3C([2H])([2H]) |
| | CN(C)CC2)═C3C═C1[2H] |
| 72 | 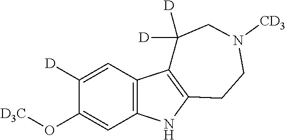 [SMILES: [2H]c1cc2c3c([nH]c2cc1OC([2H])([2H])[2H])CCN(C([2H])([2H])[2H])CC3([2H])[2H]] |
| | [2H]C1([2H])CN(C([2H])([2H]) |
| | [2H])CCC2═C1C3═C(N2)C═C |
| | (OC([2H])([2H])[2H])C([2H])═C3 |
| 73 | 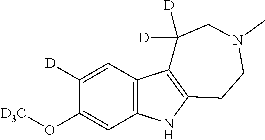 [SMILES: [2H]c1cc2c3c([nH]c2cc1OC([2H])([2H])[2H])CCN(C)CC3([2H])[2H]] |
| | CN(CC1([2H])[2H]) |
| | CCC2═C1C3═C(N2)C═C |
| | (OC([2H])([2H])[2H])C([2H])═C3 |
| 74 | 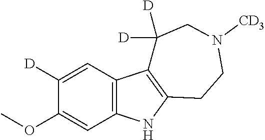 [SMILES: [2H]c1cc2c3c([nH]c2cc1OC)CCN(C([2H])([2H])[2H])CC3([2H])[2H]] |
| | COC1═CC(NC2═C3C([2H])([2H]) |
| | CN(C([2H])([2H])[2H]) |
| | CC2)═C3C═C1[2H] |
| 75 | 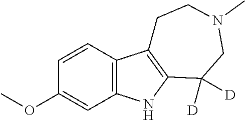 |
| | COC1═CC(NC2═C3CCN(C)CC2 |
| | ([2H])[2H])═C3C═C1 |
| 76 | 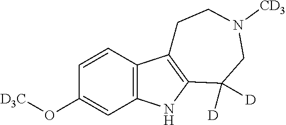 [SMILES: [2H]C([2H])([2H])Oc1ccc2c3c([nH]c2c1)C([2H])([2H])CN(C([2H])([2H])[2H])CC3] |
| | [2H]C(CN(C([2H])([2H])[2H])CC1) |
| | ([2H])C2═C1C3═C(N2)C═C(OC([2H]) |
| | ([2H])[2H])C═C3 |
| 77 | 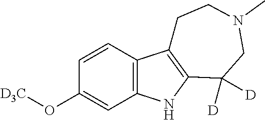 [SMILES: [2H]C([2H])([2H])Oc1ccc2c3c([nH]c2c1)C([2H])([2H])CN(C)CC3] |
| | CN(CC1)CC([2H])([2H])C2═C1C3═C |
| | (N2)C═C(OC([2H])([2H])[2H])C═C3 |
| 78 | 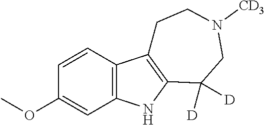 [SMILES: [2H]C1([2H])CN(C([2H])([2H])[2H])CCc2c1[nH]c1cc(OC)ccc21] |
| | COC1═CC(NC2═C3CCN(C([2H]) |
| | ([2H])[2H])CC2([2H])[2H])═C3C═C1 |
| 79 | 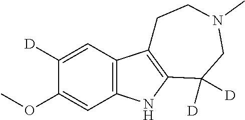 |
| | COC1═CC(NC2═C3CCN(C)CC2 |
| | ([2H])[2H])═C3C═C1[2H] |
| 80 | 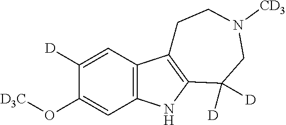 [SMILES: [2H]c1cc2c3c([nH]c2cc1OC([2H])([2H])[2H])C([2H])([2H])CN(C([2H])([2H])[2H])CC3] |
| | [2H]C(CN(C([2H])([2H])[2H]) |
| | CC1)([2H])C2═C1C3═C(N2)C═C |
| | (OC([2H])([2H])[2H])C([2H])═C3 |
| 81 | 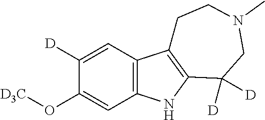 [SMILES: [2H]c1cc2c3c([nH]c2cc1OC([2H])([2H])[2H])C([2H])([2H])CN(C)CC3] |
| | CN(CC1)CC([2H])([2H]) |
| | C2═C1C3═C(N2)C═C(OC([2H])([2H]) |
| | [2H])C([2H])═C3 |
| 82 | 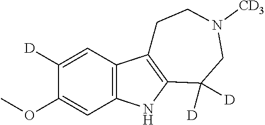 [SMILES: [2H]c1cc2c3c([nH]c2cc1OC)C([2H])([2H])CN(C([2H])([2H])[2H])CC3] |
| | COC1═CC(NC2═C3CCN(C([2H]) |
| | ([2H])[2H])CC2([2H]) |
| | [2H])═C3C═C1[2H] |
| 83 | 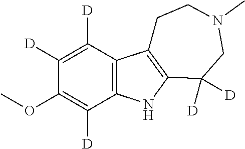 |
| | COC1═C([2H])C(NC2═C3CCN(C) |
| | CC2([2H])[2H])═C3C([2H])═C1[2H] |
| 84 | 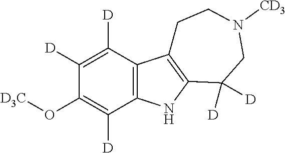 [SMILES: [2H]c1c(OC([2H])([2H])[2H])c([2H])c2[nH]c3c(c2c1[2H])CCN(C([2H])([2H])[2H])CC3([2H])[2H]] |
| | [2H]C(CN(C([2H])([2H])[2H])CC1) |
| | ([2H])C2═C1C3═C(N2)C([2H])═C |
| | (OC([2H])([2H])[2H])C([2H])═C3[2H] |
| 85 | 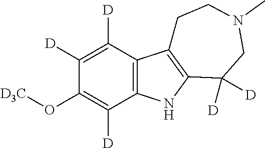 [SMILES: [2H]c1c(OC([2H])([2H])[2H])c([2H])c2[nH]c3c(c2c1[2H])CCN(C)CC3([2H])[2H]] |
| | CN(CC1)CC([2H])([2H])C2═C1C3═C |
| | (N2)C([2H])═C(OC([2H])([2H][2H]) |
| | C([2H])═C3[2H] |
| 86 | 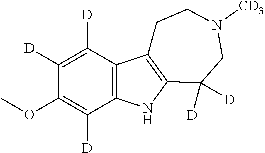 [SMILES: [2H]c1c(OC)c([2H])c2[nH]c3c(c2c1[2H])CCN(C([2H])([2H])[2H])CC3([2H])[2H]] |
| | COC1═C([2H])C(NC2═C3CCN |
| | (C([2H])([2H])[2H])CC2([2H]) |
| | [2H])═C3C([2H])═C1[2H] |
| 87 | 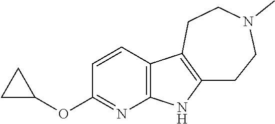 [SMILES: CN1CCc2[nH]c3nc(OC4CC4)ccc3c2CC1] |
| | CN(CC1)CCC2═C1C3═C |
| | (N2)N═C(OC4CC4)C═C3 |
| 88 | 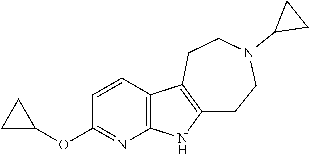 [SMILES: c1cc2c3c([nH]c2nc1OC1CC1)CCN(C1CC1)CC3] |
| | C1(OC2CC2)═NC(NC3═C4CCN |
| | (C5CC5)CC3)═C4C═C1 |
| 89 | 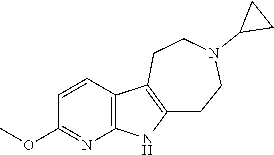 [SMILES: COc1ccc2c3c([nH]c2n1)CCN(C1CC1)CC3] |
| | COC1═NC(NC2═C3CCN(C4CC4) |
| | CC2)═C3C═C1 |
| 90 | 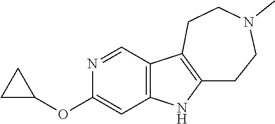 [SMILES: CN1CCc2[nH]c3cc(OC4CC4)ncc3c2CC1] |
| | CN(CC1)CCC2═C1C3═C(N2)C═C |
| | (OC4CC4)N═C3 |
| 91 | 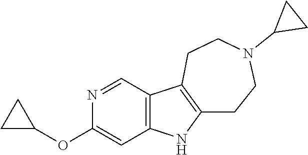 [SMILES: c1c(OC2CC2)ncc2c3c([nH]c12)CCN(C1CC1)CC3] |
| | C1(OC2CC2)═CC(NC3═C4CCN |
| | (C5CC5)CC3)═C4C═N1 |
| 92 | 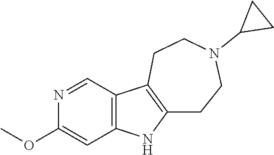 [SMILES: COc1cc2[nH]c3c(c2cn1)CCN(C1CC1)CC3] |
| | COC1═CC(NC2═C3CCN(C4CC4) |
| | CC2)═C3C═N1 |
| 93 | 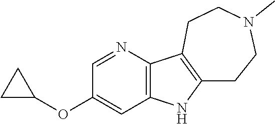 [SMILES: CN1CCc2[nH]c3cc(OC4CC4)cnc3c2CC1] |
| | CN(CC1)CCC2═C1C3═C |
| | (N2)C═C(OC4CC4)C═N3 |
| 94 | 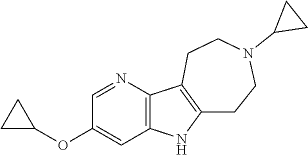 [SMILES: c1nc2c3c([nH]c2cc1OC1CC1)CCN(C1CC1)CC3] |
| | C1(OC2CC2)═CC(NC3═C4CCN |
| | (C5CC5)CC3)═C4N═C1 |
| 95 | 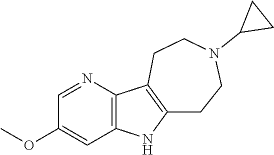 [SMILES: COc1cnc2c3c([nH]c2c1)CCN(C1CC1)CC3] |
| | COC1═CC(NC2═C3CCN(C4CC4) |
| | CC2)═C3N═C1 |
| 96 | 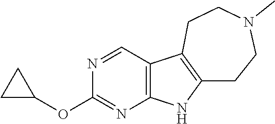 [SMILES: CN1CCc2[nH]c3nc(OC4CC4)ncc3c2CC1] |
| | CN(CC1)CCC2═C1C3═C(N2)N═C |
| | (OC4CC4)N═C3 |
| 97 | 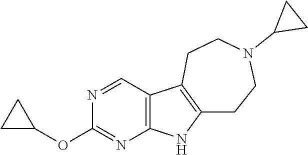 [SMILES: c1nc(OC2CC2)nc2[nH]c3c(c12)CCN(C1CC1)CC3] |
| | C1(OC2CC2)═NC(NC3═C4CCN |
| | (C5CC5)CC3)═C4C═N1 |
| 98 | 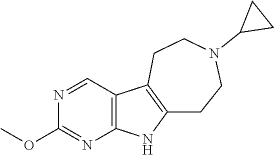 [SMILES: COc1ncc2c3c([nH]c2n1)CCN(C1CC1)CC3] |
| | COC1═NC(NC2═C3CCN(C4CC4) |
| | CC2)═C3C═N1 |
| 99 | 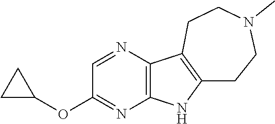 [SMILES: CN1CCc2[nH]c3nc(OC4CC4)cnc3c2CC1] |
| | CN(CC1)CCC2═C1C3═C(N2) |
| | N═C(OC4CC4)C═N3 |
| 100 | 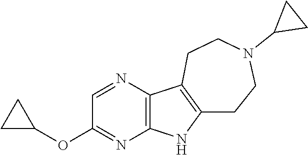 [SMILES: c1nc2c3c([nH]c2nc1OC1CC1)CCN(C1CC1)CC3] |
| | C1(OC2CC2)═NC(NC3═C4CCN |
| | (C5CC5)CC3)═C4N═C1 |
| 101 | 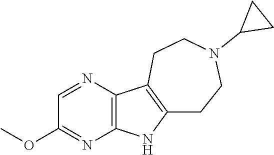 [SMILES: COc1cnc2c3c([nH]c2n1)CCN(C1CC1)CC3] |
| | COC1═NC(NC2═C3CCN(C4CC4) |
| | CC2)═C3N═C1 |
| 102 | 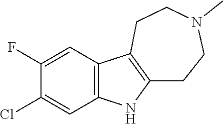 [SMILES: CN1CCc2[nH]c3cc(Cl)c(F)cc3c2CC1] |
| | C1C1═CC(NC2═C3CCN(C)CC2)═ |
| | C3C═C1F |
| 103 | 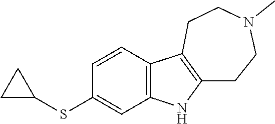 [SMILES: CN1CCc2[nH]c3cc(SC4CC4)ccc3c2CC1] |
| | CN(CC1)CCC2═C1C3═C(N2) |
| | C═C(SC4CC4)C═C3 |
| 104 | 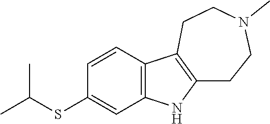 [SMILES: CC(C)Sc1ccc2c3c([nH]c2c1)CCN(C)CC3] |
| | CN(CC1)CCC2═C1C3═C(N2) |
| | C═C(SC(C)C)C═C3 |
| 105 | 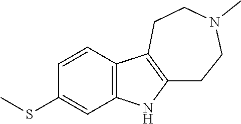 |
| | CN(CC1)CCC2═C1C3═C(N2) |
| | C═C(SC)C═C3 |
| 106 | 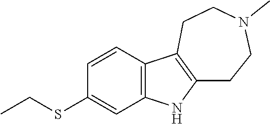 [SMILES: CCSc1ccc2c3c([nH]c2c1)CCN(C)CC3] |
| | CN(CC1)CCC2═C1C3═C(N2) |
| | C═C(SCC)C═C3 |
| 107 | 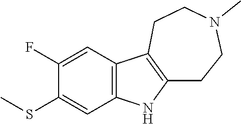 |
| | CN(CC1)CCC2═C1C3═C(N2) |
| | C═C(SC)C(F)═C3 |
| 108 | 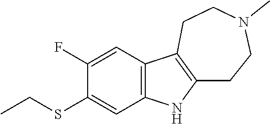 [SMILES: CCSc1cc2[nH]c3c(c2cc1F)CCN(C)CC3] |
| | CN(CC1)CCC2═C1C3═C(N2) |
| | C═C(SCC)C(F)═C3 |
| 109 | 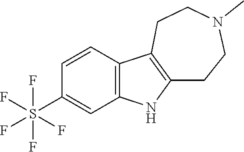 [SMILES: CN1CCc2[nH]c3cc(S(F)(F)(F)(F)F)ccc3c2CC1] |
| | CN(CC1)CCC2═C1C3═C(N2) |
| | C═C(S(F)(F)(F)(F)F)C═C3 |
| 110 | 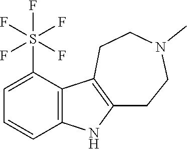 [SMILES: CN1CCc2[nH]c3cccc(S(F)(F)(F)(F)F)c3c2CC1] |
| | CN(CC1)CCC2═C1C3═C(N2) |
| | C═CC═C3S(F)(F)(F)(F)F |
| 111 | 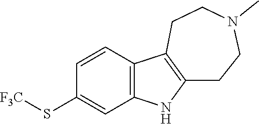 [SMILES: CN1CCc2[nH]c3cc(SC(F)(F)F)ccc3c2CC1] |
| | CN(CC1)CCC2═C1C3═C(N2) |
| | C═C(SC(F)(F)F)C═C3 |
| 112 | 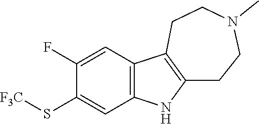 [SMILES: CN1CCc2[nH]c3cc(SC(F)(F)F)c(F)cc3c2CC1] |
| | CN(CC1)CCC2═C1C3═C(N2) |
| | C═C(SC(F)(F)F)C(F)═C3 |
| 113 | 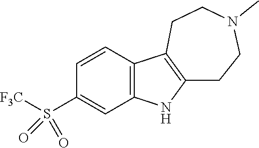 [SMILES: CN1CCc2[nH]c3cc(S(=O)(=O)C(F)(F)F)ccc3c2CC1] |
| | CN(CC1)CCC2═C1C3═C(N2) |
| | C═C(S(═O)(C(F)(F)F)═O)C═C3 |
| 114 | 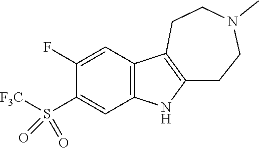 [SMILES: CN1CCc2[nH]c3cc(S(=O)(=O)C(F)(F)F)c(F)cc3c2CC1] |
| | CN(CC1)CCC2═C1C3═C(N2) |
| | C═C(S(═O)(C(F)(F)F)═O)C(F)═C3 |
| 115 | 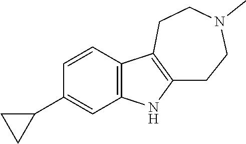 [SMILES: CN1CCc2[nH]c3cc(C4CC4)ccc3c2CC1] |
| | CN(CC1)CCC2═C1C3═C(N2) |
| | C═C(C4CC4)C═C3 |
| 116 | 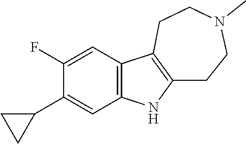 [SMILES: CN1CCc2[nH]c3cc(C4CC4)c(F)cc3c2CC1] |
| | CN(CC1)CCC2═C1C3═C(N2) |
| | C═C(C4CC4)C(F)═C3 |
| 117 | 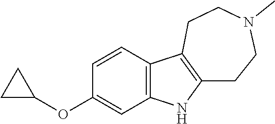 [SMILES: CN1CCc2[nH]c3cc(OC4CC4)ccc3c2CC1] |
| | CN(CC1)CCC2═C1C3═C(N2) |
| | C═C(OC4CC4)C═C3 |
| 118 | 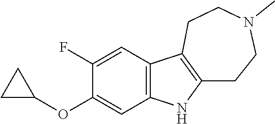 [SMILES: CN1CCc2[nH]c3cc(OC4CC4)c(F)cc3c2CC1] |
| | CN(CC1)CCC2═C1C3═C(N2) |
| | C═C(OC4CC4)C(F)═C3 |
| 119 | 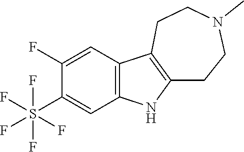 [SMILES: CN1CCc2[nH]c3cc(S(F)(F)(F)(F)F)c(F)cc3c2CC1] |
| | CN(CC1)CCC2═C1C3═C(N2) |
| | C═C(S(F)(F)(F)(F)F)C(F)═C3 |
| 120 | 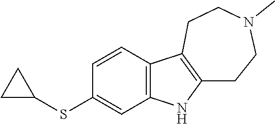 [SMILES: CN1CCc2[nH]c3cc(SC4CC4)ccc3c2CC1] |
| | CN(CC1)CCC2═C1C3═C(N2) |
| | C═C(SC4CC4)C═C3 |
| 121 | 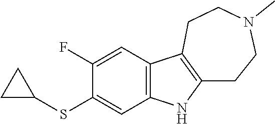 [SMILES: CN1CCc2[nH]c3cc(SC4CC4)c(F)cc3c2CC1] |
| | CN(CC1)CCC2═C1C3═C(N2) |
| | C═C(SC4CC4)C(F)═C3 |
| 122 | 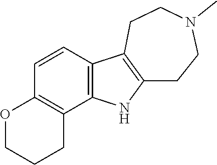 [SMILES: CN1CCc2[nH]c3c4c(ccc3c2CC1)OCCC4] |
| | CN(CC1)CCC2═C1C3═C(N2) |
| | C4═C(OCCC4)C═C3 |
| 123 | 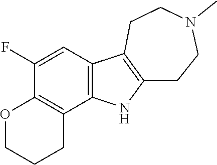 [SMILES: CN1CCc2[nH]c3c4c(c(F)cc3c2CC1)OCCC4] |
| | CN(CC1)CCC2═C1C3═C(N2) |
| | C4═C(OCCC4)C(F)═C3 |
| 124 | 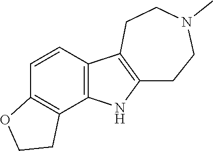 [SMILES: CN1CCc2[nH]c3c4c(ccc3c2CC1)OCC4] |
| | CN(CC1)CCC2═C1C3═C(N2) |
| | C4═C(OCC4)C═C3 |
| 125 | 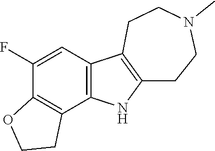 [SMILES: CN1CCc2[nH]c3c4c(c(F)cc3c2CC1)OCC4] |
| | CN(CC1)CCC2═C1C3═C(N2) |
| | C4═C(OCC4)C(F)═C3 |
| 126 | 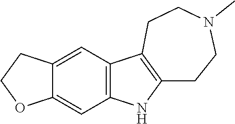 [SMILES: CN1CCc2[nH]c3cc4c(cc3c2CC1)CCO4] |
| | CN(CC1)CCC2═C1C3═C(N2) |
| | C═C4C(CCO4)═C3 |
| 127 | 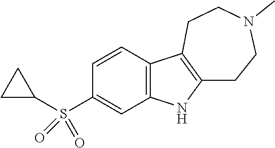 [SMILES: CN1CCc2[nH]c3cc(S(=O)(=O)C4CC4)ccc3c2CC1] |
| | CN(CC1)CCC2═C1C3═C(N2)C═C |
| | (S(C4CC4)(═O)═O)C═C3 |
| 128 | 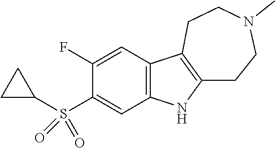 [SMILES: CN1CCc2[nH]c3cc(S(=O)(=O)C4CC4)c(F)cc3c2CC1] |
| | CN(CCl)CCC2═C1C3═C(N2)C═C |
| | (S(C4CC4)(═O)═O)C(F)═C3 |
| 129 | 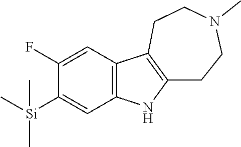 |
| | CN(CC1)CCC2═C1C3═C(N2)C═C |
| | ([Si](C)(C)C)C═C3 |
| 130 | 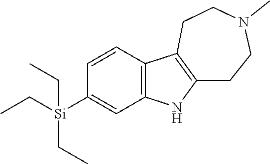 [SMILES: CC[Si](CC)(CC)c1ccc2c3c([nH]c2c1)CCN(C)CC3] |
| | CN(CC1)CCC2═C1C3═C(N2)C═C |
| | ([Si](CC)(CC)CC)C═C3 |
| 131 | 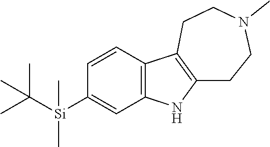 [SMILES: CN1CCc2[nH]c3cc([Si](C)(C)C(C)(C)C)ccc3c2CC1] |
| | CN(CC1)CCC2═C1C3═C(N2)C═ |
| | C([Si](C)(C)C(C)(C)C)C═C3 |
| 132 | 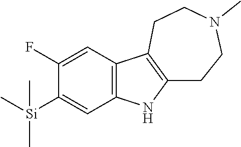 |
| | CN(CC1)CCC2═C1C3═C(N2)C═C |
| | ([Si](C)(C)C)C(F)═C3 |
| 133 | 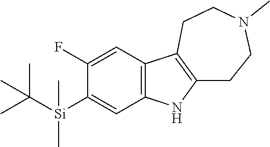 [SMILES: CN1CCc2[nH]c3cc([Si](C)(C)C(C)(C)C)c(F)cc3c2CC1] |
| | CN(CC1)CCC2═C1C3═C(N2)C═C |
| | ([Si](C)(C)C(C)(C)C)C(F)═C3 |
| 134 | 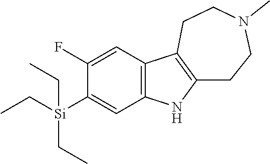 [SMILES: CC[Si](CC)(CC)c1cc2[nH]c3c(c2cc1F)CCN(C)CC3] |
| | CN(CC1)CCC2═C1C3═C(N2)C═C |
| | ([Si](CC)(CC)CC)C(F)═C3 |
| 135 | 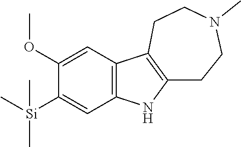 |
| | CN(CC1)CCC2═C1C3═C(N2)C═C |
| | ([Si](C)(C)C)C(OC)═C3 |
| 136 | 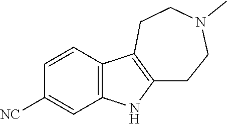 [SMILES: CN1CCc2[nH]c3cc(C#N)ccc3c2CC1] |
| | CN(CC1)CCC2═C1C3═C(N2)C═C |
| | (C#N)C═C3 |
| 137 | 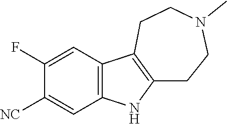 [SMILES: CN1CCc2[nH]c3cc(C#N)c(F)cc3c2CC1] |
| | CN(CC1)CCC2═C1C3═C(N2)C═C |
| | (C#N)C(F)═C3 |
| 138 | 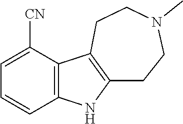 [SMILES: CN1CCc2[nH]c3cccc(C#N)c3c2CC1] |
| | CN(CC1)CCC2═C1C3═C(N2)C═C |
| | C═C3C#N |
| 139 | 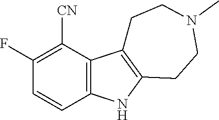 [SMILES: CN1CCc2[nH]c3ccc(F)c(C#N)c3c2CC1] |
| | CN(CC1)CCC2═C1C3═C(N2)C═ |
| | CC(F)═C3C#N |
| 140 | 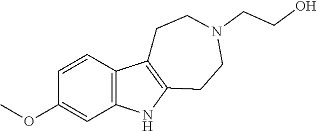 [SMILES: COc1ccc2c3c([nH]c2c1)CCN(CCO)CC3] |
| | OCCN(CC1)CCC2═C1C3═C(N2) |
| | C═C(OC)C═C3 |
| 141 | 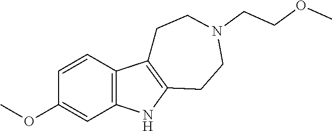 |
| | COC1═CC(NC2═C3CCN(CCOC) |
| | CC2)═C3C═C1 |
| 142 | 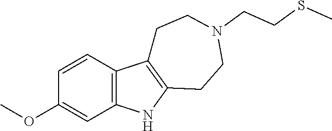 |
| | COC1═CC(NC2═C3CCN(CCSC) |
| | CC2)═C3C═C1 |
| 143 | 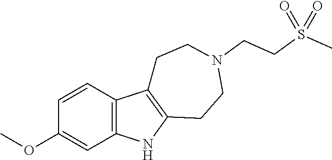 |
| | COCl═CC(NC2═C3CCN(CCS(C) |
| | (═O)═O)CC2)═C3C═C1 |
| 144 | 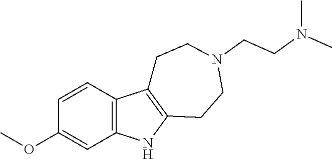 |
| | COC1═CC(NC2═C3CCN(CCN(C) |
| | C)CC2)═C3C═C1 |
| 145 | 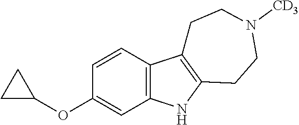 [SMILES: [2H]C([2H])([2H])N1CCc2[nH]c3cc(OC4CC4)ccc3c2CC1] |
| | [2H]C([2H])([2H])N(CC1) |
| | CCC2═C1C3═C(N2)C═C |
| | (OC4CC4)C═C |
| 146 | 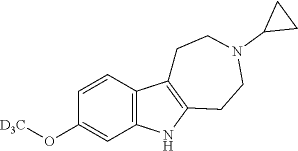 [SMILES: [2H]C([2H])([2H])Oc1ccc2c3c([nH]c2c1)CCN(C1CC1)CC3] |
| | [2H]C([2H])([2H])OC1═CC |
| | (NC2═C3CCN(C4CC4) |
| | CC2)═C3C═C1 |
| 147 | 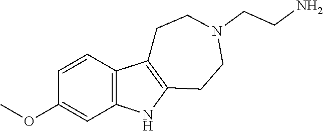 [SMILES: COc1ccc2c3c([nH]c2c1)CCN(CCN)CC3] |
| | NCCN(CC1)CCC2═C1C3═C(N2) |
| | C═C(OC)C═C3 |
| 148 | 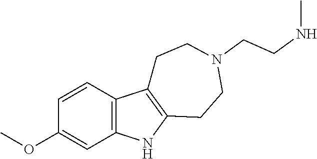 |
| | COC1═CC(NC2═C3CCN(CCNC) |
| | CC2)═C3C═C1 |
| 149 | 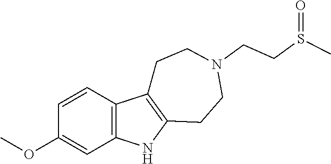 |
| | COC1═CC(NC2═C3CCN(CCS |
| | (C)═O)CC2)═C3C═C1 |
| 150 | 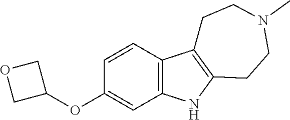 [SMILES: CN1CCc2[nH]c3cc(OC4COC4)ccc3c2CC1] |
| | CN(CC1)CCC2═C1C3═C(N2)C═ |
| | C(OC4COC4)C═C3 |
| 151 | 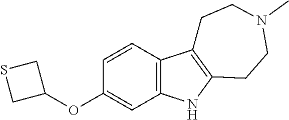 [SMILES: CN1CCc2[nH]c3cc(OC4CSC4)ccc3c2CC1] |
| | CN(CC1)CCC2═C1C3═C(N2)C═C( |
| | OC4CSC4)C═C3 |
| 152 | 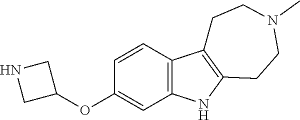 [SMILES: CN1CCc2[nH]c3cc(OC4CNC4)ccc3c2CC1] |
| | CN(CC1)CCC2═C1C3═C(N2)C═C |
| | (OC4CNC4)C═C3 |
| 153 | 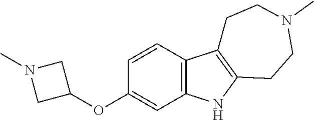 |
| | CN(CC1)CCC2═C1C3═C(N2)C═C |
| | (OC4CN(C)C4)C═C3 |
| 154 | 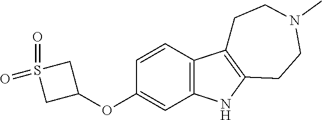 [SMILES: CN1CCc2[nH]c3cc(OC4CS(=O)(=O)C4)ccc3c2CC1] |
| | CN(CC1)CCC2═C1C3═C(N2)C═C |
| | (OC4CS(C4)(═O)═O)C═C3 |
| 155 | 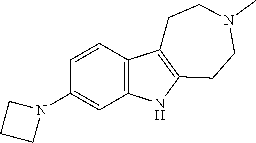 [SMILES: CN1CCc2[nH]c3cc(N4CCC4)ccc3c2CC1] |
| | CN(CC1)CCC2═C1C3═C(N2)C═C |
| | (N4CCC4)C═C3 |
| 156 | 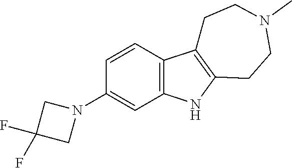 [SMILES: CN1CCc2[nH]c3cc(N4CC(F)(F)C4)ccc3c2CC1] |
| | CN(CC1)CCC2═C1C3═C(N2)C═C |
| | (N4CC(F)(F)C4)C═C3 |
| 157 | 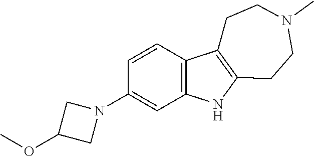 |
| | CN(CC1)CCC2═C1C3═C(N2)C═C |
| | (N4CC(OC)C4)C═C3 |
| 158 | 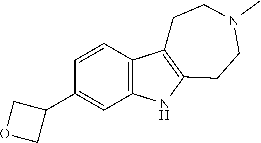 [SMILES: CN1CCc2[nH]c3cc(C4COC4)ccc3c2CC1] |
| | CN(CC1)CCC2═C1C3═C(N2)C═C |
| | (C4COC4)C═C3 |
| 159 | 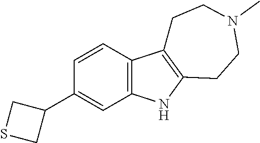 [SMILES: CN1CCc2[nH]c3cc(C4CSC4)ccc3c2CC1] |
| | CN(CC1)CCC2═C1C3═C(N2)C═C |
| | (C4CSC4)C═C3 |
| 160 | 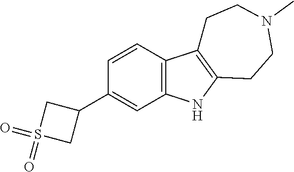 [SMILES: CN1CCc2[nH]c3cc(C4CS(=O)(=O)C4)ccc3c2CC1] |
| | CN(CC1)CCC2═C1C3═C(N2)C═C |
| | (C4CS(C4)(═O)═O)C═C3 |
| 161 | 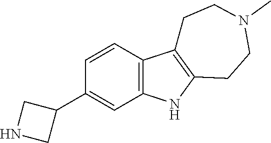 [SMILES: CN1CCc2[nH]c3cc(C4CNC4)ccc3c2CC1] |
| | CN(CC1)CCC2═C1C3═C(N2)C═C( |
| | C4CNC4)C═C3 |
| 162 | 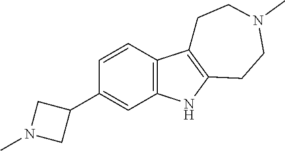 |
| | CN(CC1)CCC2═C1C3═C(N2)C═C |
| | (C4CN(C)C4)C═C3 |
| 163 | 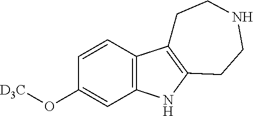 [SMILES: [2H]C([2H])([2H])Oc1ccc2c3c([nH]c2c1)CCNCC3] |
| | [2H]C([2H])([2H])OC1═CC |
| | (NC2═C3CCNCC2)═C3C═C1 |
| 164 | 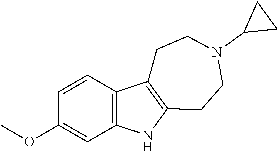 [SMILES: COc1ccc2c3c([nH]c2c1)CCN(C1CC1)CC3] |
| | COC1═CC(NC2═C3CCN(C4CC4) |
| | CC2)═C3C═C1 |
| 165 | 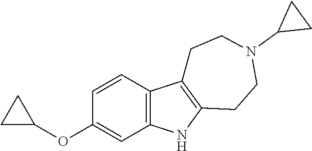 [SMILES: c1cc2c3c([nH]c2cc1OC1CC1)CCN(C1CC1)CC3] |
| | C1(OC2CC2)═CC(NC3═C4CCN |
| | (C5CC5)CC3)═C4C═C1 |
| 166 | 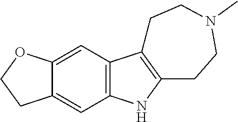 [SMILES: CN1CCc2[nH]c3cc4c(cc3c2CC1)OCC4] |
| | CN(CC1)CCC2═C1C3═C(N2)C═C |
| | 4C(OCC4)═C3 |
| 167 | 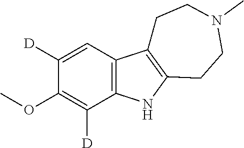 |
| | COC1═C([2H])C(NC2═C3CCN(C) |
| | CC2)═C3C═C1[2H] |

### Pharmaceutically Acceptable Salts:

The present disclosure provides for pharmaceutically-acceptable salts of any compound described herein as well as the use of such salts. As is understood by those of skill in the art, any compound with an ionizable group, such as an acidic hydrogen, or a basic nitrogen, can be provided in the form of a salt, and pharmaceutically acceptable salt forms of such compounds are specifically contemplated herein. Pharmaceutically-acceptable salts include, for example, acid-addition salts and base-addition salts. The acid that is added to the compound to form an acid-addition salt can be an organic acid or an inorganic acid. A base that is added to the compound to form a base-addition salt can be an organic base or an inorganic base. In some embodiments, a pharmaceutically-acceptable salt is a metal salt. In some embodiments, a pharmaceutically-acceptable salt is an ammonium salt.

Metal salts can arise from the addition of an inorganic base to a compound of the present disclosure having an acidic functional group. The inorganic base consists of a metal cation paired with a basic counterion, such as, for example, hydroxide, carbonate, bicarbonate, or phosphate. The metal can be an alkali metal, alkaline earth metal, transition metal, or main group metal. In some embodiments, the metal is a metal cation, such as lithium, sodium, potassium, cesium, cerium, magnesium, manganese, iron, calcium, strontium, cobalt, titanium, aluminum, copper, cadmium, or zinc.

In some embodiments, a metal salt is a lithium salt, a sodium salt, a potassium salt, a cesium salt, a cerium salt, a magnesium salt, a manganese salt, an iron salt, a calcium salt, a strontium salt, a cobalt salt, a titanium salt, an aluminum salt, a copper salt, a cadmium salt, or a zinc salt.

Ammonium salts can arise from the addition of ammonia or an organic amine to a compound of the present disclosure. In some embodiments, the organic amine is trimethyl amine, triethyl amine, diisopropyl amine, ethanol amine, diethanol amine, triethanol amine, morpholine, N-methylmorpholine, piperidine, N-methylpiperidine, N-ethylpiperidine, dibenzylamine, piperazine, pyridine, pyrazole, pyrazolidine, pyrazoline, pyridazine, pyrimidine, imidazole, or pyrazine.

In some embodiments, an ammonium salt is a triethyl amine salt, trimethyl amine salt, a diisopropyl amine salt, an ethanolamine salt, a diethanol amine salt, a triethanol amine salt, a morpholine salt, an N-methylmorpholine salt, a piperidine salt, an N-methylpiperidine salt, an N-ethylpiperidine salt, a dibenzylamine salt, a piperazine salt, a pyridine salt, a pyrazole salt, a pyridazine salt, a pyrimidine salt, an imidazole salt, or a pyrazine salt.

Acid addition salts can arise from the addition of an acid to a compound of the present disclosure that includes a basic functional group. In some embodiments, the acid is organic. In other embodiments, the acid is inorganic. In some embodiments, the acid is hydrochloric acid, hydrobromic acid, hydroiodic acid, nitric acid, nitrous acid, sulfuric acid, a phosphoric acid, isonicotinic acid, lactic acid, salicylic acid, tartaric acid, ascorbic acid, gentisic acid, gluconic acid, glucuronic acid, saccharic acid, formic acid, benzoic acid, glutamic acid, pantothenic acid, acetic acid, propionic acid, butyric acid, fumaric acid, succinic acid, methanesulfonic acid, ethanesulfonic acid, benzenesulfonic acid, p-toluenesulfonic acid, citric acid, oxalic acid, maleic acid or xinafoic acid.

In some embodiments, the salt is a hydrochloride salt, a hydrobromide salt, a hydroiodide salt, a nitrate salt, a nitrite salt, a sulfate salt, a sulfite salt, a phosphate salt, isonicotinate salt, a lactate salt, a salicylate salt, a tartrate salt, an ascorbate salt, a gentisate salt, a gluconate salt, a glucuronate salt, a saccharate salt, a formate salt, a benzoate salt, a glutamate salt, a pantothenate salt, an acetate salt, a propionate salt, a butyrate salt, a fumarate salt, a succinate salt, a methanesulfonate salt, an ethanesulfonate salt, a benzenesulfonate salt, ap-toluenesulfonate salt, a citrate salt, an oxalate salt, a maleate salt or a xinafoate salt.

### Pharmaceutical Compositions and Formulations

In some embodiments, the present invention provides a pharmaceutical composition comprising a compound of the present invention, such as a composition comprising a compound of Formula I, I-1, I-1A, I-1B, II, IIa, IIb, IIc, IId, IIe, IIf, IIg or a pharmaceutically acceptable salt thereof, illustrated above, and a pharmaceutically acceptable excipient. Such compositions are suitable for administration to a subject, such as a human subject.

In some embodiments, the present invention provides a pharmaceutical composition comprising a compound of the present invention and a pharmaceutically acceptable carrier, adjuvant, or vehicle.

The presently disclosed pharmaceutical compositions can be prepared in a wide variety of oral, parenteral and topical dosage forms. Oral preparations include tablets, pills, powder, capsules, liquids, lozenges, cachets, gels, syrups, slurries, suspensions, etc., suitable for ingestion by the patient. The compositions of the present invention can also be administered by injection, that is, intravenously, intramuscularly, intracutaneously, subcutaneously, intraduodenally, or intraperitoneally. Also, the compositions described herein can be administered by inhalation, for example, intranasally. Additionally, the compositions of the present invention can be administered transdermally. The compositions of this invention can also be administered by intraocular, intravaginal, and intrarectal routes including suppositories, insufflation, powders and aerosol formulations (for examples of steroid inhalants, see Rohatagi, J. Clin. Pharmacol. 35:1187-1193, 1995; Tjwa, Ann. Allergy Asthma Immunol. 75:107-111, 1995). Accordingly, the present invention also provides pharmaceutical compositions including a pharmaceutically acceptable carrier or excipient and the compounds of the present invention.

In some embodiments, the composition is a transmucosal formulation.

For preparing pharmaceutical compositions from the compounds disclosed herein, pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, binders, preservatives, tablet disintegrating agents, or an encapsulating material. Details on techniques for formulation and administration are well described in the scientific and patent literature, see, e.g., the latest edition of Remington's Pharmaceutical Sciences, Mack Publishing Co, Easton PA (“Remington's”). In powders, the carrier is a finely divided solid, which is in a mixture with the finely divided active component. In tablets, the active component is mixed with the carrier having the necessary binding properties in suitable proportions and compacted in the shape and size desired.

The powders and tablets preferably contain from 5% to 70% or 10% to 70% of the compounds of the present invention.

Suitable solid excipients include, but are not limited to, magnesium carbonate; magnesium stearate; talc; pectin; dextrin; starch; tragacanth; a low melting wax; cocoa butter; carbohydrates; sugars including, but not limited to, lactose, sucrose, mannitol, or sorbitol, starch from com, wheat, rice, potato, or other plants; cellulose such as methyl cellulose, hydroxypropylmethylcellulose, or sodium carboxymethylcellulose; and gums including arabic and tragacanth; as well as proteins including, but not limited to, gelatin and collagen.

If desired, disintegrating or solubilizing agents may be added, such as the cross-linked polyvinyl pyrrolidone, agar, alginic acid, or a salt thereof, such as sodium alginate.

For preparing suppositories, a low melting wax, such as a mixture of fatty acid glycerides or cocoa butter, is first melted and the compounds of the present invention are dispersed homogeneously therein, as by stirring. The molten homogeneous mixture is then poured into convenient sized molds, allowed to cool, and thereby to solidify.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinyl pyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings, release controlling coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. In solid dosage forms, the active compound may be admixed with at least one inert diluent such as sucrose, lactose or starch. Such dosage forms may also comprise, as is normal practice, additional substances other than inert diluents, e.g, tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the compounds of the present invention in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, com, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Oil suspensions can be formulated by suspending the compound of the present invention in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations of the invention can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

Therapeutic agents can be in micro-encapsulated form with one or more excipients.

The compositions of the present invention can also be delivered as microspheres for slow release in the body. For example, microspheres can be formulated for administration via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). Both transdermal and intradermal routes afford constant delivery for weeks or months.

In some embodiments, the pharmaceutical compositions of the present invention can be formulated for parenteral administration, such as intravenous (IV) administration or administration into a body cavity or lumen of an organ. The formulations for administration will commonly comprise a solution of the compositions of the present invention dissolved in a pharmaceutically acceptable carrier. Among the acceptable vehicles and solvents that can be employed are water and Ringer's solution, U.S.P. and isotonic sodium chloride solution. In addition, sterile fixed oils can conventionally be employed as a solvent or suspending medium. For this purpose, any bland fixed oil can be employed including synthetic mono- or diglycerides. In addition, fatty acids such as oleic acid and its glyceride derivatives can likewise be used in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

These solutions are sterile and generally free of undesirable matter. These formulations may be sterilized by conventional, well-known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of the compositions of the present invention in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution, suspension or emulsion in a nontoxic parenterally-acceptable diluent or solvent, such as a solution of 1,3-butanediol.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

To prolong the effect of a compound of the present disclosure, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

### Administration:

The compositions of the present disclosure may be administered orally, parenterally, enterally, intracisternally, intraperitoneally, by inhalation spray, topically, rectally, nasally, buccally, vaginally or via an implanted reservoir. In some embodiments, the composition is administered orally, intraperitoneally, or intravenously.

Pharmaceutically acceptable compositions may be orally administered in any orally acceptable dosage form including, but not limited to, capsules, tablets, aqueous suspensions or solutions. In the case of tablets for oral use, carriers commonly used include lactose and com starch. Lubricating agents, such as magnesium stearate, may also be added. For oral administration in a capsule form, useful diluents include lactose and dried corn starch. When aqueous suspensions are required for oral use, the active ingredient is combined with emulsifying and suspending agents. If desired, certain sweetening, flavoring or coloring agents may also be added.

Orally administered formulations may be administered with or without food. In some embodiments, the pharmaceutically acceptable composition is administered orally without food. In other embodiments, the pharmaceutically acceptable composition is administered orally with food.

In some embodiments, the pharmaceutically acceptable composition is administered without food. In other embodiments, the pharmaceutically acceptable composition is administered with food.

The compositions of the present invention can be administered by any suitable means, including oral, parenteral and topical methods. Transdermal administration methods, by a topical route, can be formulated as applicator sticks, solutions, suspensions, emulsions, gels, creams, ointments, pastes, jellies, paints, powders, and aerosols.

Alternatively, pharmaceutically acceptable compositions can be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Compositions for rectal or vaginal administration are preferably suppositories which can be prepared by mixing the compounds of this disclosure with suitable non-irritating excipients or carriers such as cocoa butter, polyethylene glycol or a suppository wax which are solid at ambient temperature but liquid at body temperature and therefore melt in the rectum or vaginal cavity and release the active compound.

Dosage forms for topical or transdermal administration of a compound of this disclosure include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this disclosure.

Additionally, the present disclosure contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

In some embodiments, the formulations of the compositions of the present invention can be delivered by the use of liposomes which fuse with the cellular membrane or are endocytosed, for example, by employing ligands attached to the liposome, or attached directly to the oligonucleotide, that bind to surface membrane protein receptors of the cell resulting in endocytosis. By using liposomes, particularly where the liposome surface carries ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989).

The pharmaceutical preparation is preferably in unit dosage form. In such form the preparation is subdivided into unit doses containing appropriate quantities of the compounds of the present invention. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The compound of the present invention can be present in any suitable amount, and can depend on various factors including, but not limited to, weight and age of the subject, state of the disease, and the like as is known to those of ordinary skill in the art. Suitable dosage ranges for the compounds disclosed herein include from about 0.1 mg to about 2,000 mg, or about 1 mg to about 1000 mg, or about 10 mg to about 750 mg, or about 25 mg to about 500 mg, or about 50 mg to about 250 mg or about 500 mg to about 800 mg. Suitable dosages for the compound of the present invention include about 1 mg, 5, 10, 20, 30, 40, 50, 60, 70, 80, 90, 100, 200, 300, 400, 500, 600, 700, 800, 900 or 1000 mg.

The compounds disclosed herein can be administered at any suitable frequency, interval and duration. For example, the compounds can be administered once an hour, or two, three or more times an hour, once a day, or two, three, or more times per day, or once every 2, 3, 4, 5, 6, or 7 days, so as to provide the preferred dosage level. When the compound of the present invention is administered more than once a day, representative intervals include 5, 10, 15, 20, 30, 45 and 60 minutes, as well as 1, 2, 4, 6, 8, 10, 12, 16, 20, and 24 hours. The compound of the present invention can be administered once, twice, or three or more times, for an hour, for 1 to 6 hours, for 1 to 12 hours, for 1 to 24 hours, for 6 to 12 hours, for 12 to 24 hours, for a single day, for 1 to 7 days, for a single week, for 1 to 4 weeks, for a month, for 1 to 12 months, for a year or more, or even indefinitely.

The composition can also contain other compatible therapeutic agents. The compounds described herein can be used in combination with one another, with other active agents known to be useful in modulating a glucocorticoid receptor, or with adjunctive agents that may not be effective alone, but may contribute to the efficacy of the active agent.

The compounds of the present invention can be co-administered with a second active agent. Co-administration includes administering the compound of the present invention and active agent within 0.5, 1, 2, 4, 6, 8, 10, 12, 16, 20, or 24 hours of each other. Co-administration also includes administering the compound of the present invention and active agent simultaneously, approximately simultaneously (e.g., within about 1, 5, 10, 15, 20, or 30 minutes of each other), or sequentially in any order. Moreover, the compound of the present invention and the active agent can each be administered once a day, or two, three, or more times per day so as to provide the preferred dosage level per day.

In some embodiments, co-administration can be accomplished by co-formulation, such as by preparing a single pharmaceutical composition including both the compound of the present invention and a second active agent. In other embodiments, the compound of the present invention and the second active agent can be formulated separately.

The disclosed compounds and the second active agent can be present in the compositions of the present invention in any suitable weight ratio, such as from about 1:100 to about 100:1 (w/w), or about 1:50 to about 50:1, or about 1:25 to about 25:1, or about 1:10 to about 10:1, or about 1:5 to about 5:1 (w/w). The compound of the present invention and the second active agent can be present in any suitable weight ratio, such as about 1:100 (w/w), 1:50, 1:25, 1:10, 1:5, 1:4, 1:3, 1:2, 1:1, 2:1, 3:1, 4:1, 5:1, 10:1, 25:1, 50:1 or 100:1 (w/w). Other dosages and dosage ratios of the compound of the present invention and the active agent are suitable in the compositions and methods disclosed herein.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated.

### Methods of Treatment

In yet another aspect, the present disclosure provides a method of treating or preventing a disease, disorder, or condition in which an increased level of a tryptamine psychedelic such as a phenethylamine analog disclosed herein is beneficial, comprising administering to a subject in need thereof an effective amount of a compound selected from those of Formulas I, I-1, I-1A, I-1, II, IIa, IIb, IIc, IId, IIe, IIf, IIg, Table 1, or a pharmaceutically acceptable salt thereof. In some embodiments, the condition comprises post-traumatic stress disorder, major depression, schizophrenia, or substance abuse. Additional examples of methods for using the disclosed compounds are described below.

The compounds of the present disclosure, such compounds of Formula I, I-1, I-1A, I-1B, II, IIa, IIb, IIc, IId, IIe, IIf, IIg, Table 1, or pharmaceutically acceptable salts thereof can be used for increasing neuronal plasticity. The compounds of the present invention can also be used to treat any brain disease. The compounds of the present invention can also be used for increasing at least one of translation, transcription or secretion of neurotrophic factors.

In some embodiments, a compound of the present disclosure, such as a compound of Formula I, I-1, I-1A, I-1B, II, IIa, IIb, IIc, IId, IIe, IIf, IIg, Table 1, or a pharmaceutically acceptable salt is used to treat neurological diseases. In some embodiments, the compounds have, for example, anti-addictive properties, antidepressant properties, anxiolytic properties, or a combination thereof. In some embodiments, the neurological disease is a neuropsychiatric disease. In some embodiments, the neuropsychiatric disease is a mood or anxiety disorder. In some embodiments, the neurological disease is a migraine, headaches (e.g., cluster headache), post-traumatic stress disorder (PTSD), anxiety, depression, neurodegenerative disorder, Alzheimer's disease, Parkinson's disease, psychological disorder, treatment resistant depression, suicidal ideation, major depressive disorder, bipolar disorder, schizophrenia, stroke, traumatic brain injury, and addiction (e.g., substance use disorder). In some embodiments, the neurological disease is a migraine or cluster headache.

In some embodiments, the neurological disease is a neurodegenerative disorder, Alzheimer's disease, or Parkinson's disease.

In some embodiments, the brain disorder comprises treatment resistant depression, suicidal ideation, major depressive disorder, bipolar disorder, schizophrenia, stroke, traumatic brain injury, substance use disorder and/or anxiety. In some embodiments, the brain disorder is treatment resistant depression, suicidal ideation, major depressive disorder, bipolar disorder, schizophrenia, or substance use disorder. In some embodiments, the brain disorder is stroke or traumatic brain injury. In some embodiments, the brain disorder is treatment resistant depression, suicidal ideation, major depressive disorder, bipolar disorder, or substance use disorder. In some embodiments, the brain disorder is schizophrenia. In some embodiments, the brain disorder is alcohol use disorder.

In some embodiments, the methods described herein are for treating a disease or disorder that is a neurological disease. For example, a compound provided herein can exhibit, anti-addictive properties, antidepressant properties, anxiolytic properties, or a combination thereof. In some embodiments, the neurological disease is a neuropsychiatric disease. In some embodiments, the neuropsychiatric disease is a mood or anxiety disorder. In some embodiments, the neurological disease is selected from migraine, headaches (e.g., cluster headache), post-traumatic stress disorder (PTSD), anxiety, depression, neurodegenerative disorder, Alzheimer's disease, Parkinson's disease, psychological disorder, treatment resistant depression, suicidal ideation, major depressive disorder, bipolar disorder, schizophrenia, stroke, hypoxic brain injury, chronic traumatic encephalopathy (CTE), traumatic brain injury, dementia, and addiction (e.g., substance use disorder). In some embodiments, the neurological disease is a migraine or cluster headache. In some embodiments, the neurological disease is a neurodegenerative disorder, dementia, Alzheimer's disease, or Parkinson's disease. In some embodiments, the neurological disease is dementia. In some embodiments, the neurological disease is a psychological disorder, treatment resistant depression, suicidal ideation, major depressive disorder, bipolar disorder, schizophrenia, post-traumatic stress disorder (PTSD), addiction (e.g., substance use disorder), depression, or anxiety. In some embodiments, the neuropsychiatric disease is a psychological disorder, treatment resistant depression, suicidal ideation, major depressive disorder, bipolar disorder, schizophrenia, post-traumatic stress disorder (PTSD), addiction (e.g., substance use disorder), depression, or anxiety. In some embodiments, the neuropsychiatric disease or neurological disease is post-traumatic stress disorder (PTSD), addiction (e.g., substance use disorder), schizophrenia, depression, or anxiety. In some embodiments, the neuropsychiatric disease or neurological disease is addiction (e.g., substance use disorder). In some embodiments, the neuropsychiatric disease or neurological disease is depression. In some embodiments, the neuropsychiatric disease or neurological disease is anxiety. In some embodiments, the neuropsychiatric disease or neurological disease is post-traumatic stress disorder (PTSD). In some embodiments, the neurological disease is stroke or traumatic brain injury. In some embodiments, the neuropsychiatric disease or neurological disease is schizophrenia.

In some embodiments, a compound of the present invention is used for increasing neuronal plasticity. In some embodiments, the compounds described herein are used for treating a brain disorder. In some embodiments, the compounds described herein are used for increasing at least one of translation, transcription, or secretion of neurotrophic factors.

In some embodiments, the compounds of the present invention, such as a compound of Formula I, I-1, I-1A, I-1B, II, IIa, IIb, IIc, IId, IIe, IIf, IIg, Table 1, or pharmaceutically acceptable salts thereof have activity as 5-HT_{2A} modulators. In some embodiments, the compounds of the present invention elicit a biological response by activating the 5-HT_{2A} receptor (e.g., through allosteric modulation or modulation of a biological target that activates the 5-HT_{2A} receptor). 5-HT_{2A} agonism has been correlated with the promotion of neural plasticity (Ly et al., 2018). 5-HT_{2A} antagonists abrogate the neuritogenesis and spinogenesis effects of hallucinogenic compounds with 5-HT_{2A} agonist activity, for example, DMT, LSD, and DOI. In some embodiments, the compounds of the present invention are 5-HT_{2A} modulators and promote neural plasticity (e.g., cortical structural plasticity). In some embodiments, the compounds of the present invention are selective 5-HT_{2A} modulators and promote neural plasticity (e.g., cortical structural plasticity). In some embodiments, promotion of neural plasticity includes, for example, increased dendritic spine growth, increased synthesis of synaptic proteins, strengthened synaptic responses, increased dendritic arbor complexity, increased dendritic branch content, increased spinogenesis, increased neuritogenesis, or any combination thereof. In some embodiments, increased neural plasticity includes, for example, increased cortical structural plasticity in the anterior parts of the brain.

In some embodiments, the 5-HT_{2A} modulators (e.g., 5-HT_{2A} agonists) are non-hallucinogenic. In some embodiments, the non-hallucinogenic 5-HT_{2A} modulators (e.g., 5-HT_{2A} agonists) are used to treat neurological diseases, which modulators do not elicit dissociative side-effects. In some embodiments, the hallucinogenic potential of the compounds described herein is assessed in vitro. In some embodiments, the hallucinogenic potential assessed in vitro of the compounds described herein is compared to the hallucinogenic potential assessed in vitro of hallucinogenic homologs. In some embodiments, the compounds described herein elicit less hallucinogenic potential in vitro than the hallucinogenic homologs.

In some embodiments, the presently disclosed compounds are serotonin receptor modulators, such as modulators of serotonin receptor 2A (5-HT_{2A} modulators, e.g., 5-HT_{2A} agonists), that are used to treat a brain disorder. The presently disclosed compounds of Formulas I, I-1, I-1A, I-1B, II, IIa, IIb, IIc, IId, IIe, IIf, IIg, Table 1, and pharmaceutically acceptable salts thereof can function as 5-HT_{2A} agonists alone, or in combination with a second therapeutic agent that also is a 5-HT_{2A} modulator. In such cases the second therapeutic agent can be an agonist or an antagonist. In some instances, it may be helpful administer a 5-HT_{2A} antagonist in combination with a compound of the present invention to mitigate undesirable effects of 5-HT_{2A} agonism, such as potential hallucinogenic effects. Serotonin receptor modulators useful as second therapeutic agents for combination therapy as described herein are known to those of skill in the art and include, without limitation, ketanserin, volinanserin (MDL-100907), eplivanserin (SR-46349), pimavanserin (ACP-103), glemanserin (MDL-11939), ritanserin, flibanserin, nelotanserin, blonanserin, mianserin, mirtazapine, roluperiodone (CYR-101, MIN-101), quetiapine, olanzapine, altanserin, acepromazine, nefazodone, risperidone, pruvanserin, AC-90179, AC-279, adatanserin, fananserin, HY10275, benanserin, butanserin, manserin, iferanserin, lidanserin, pelanserin, seganserin, tropanserin, lorcaserin, ICI-169369, methiothepin, methysergide, trazodone, cinitapride, cyproheptadine, brexpiprazole, cariprazine, agomelatine, setoperone, 1-(1-Naphthyl)piperazine, LY-367265, pirenperone, metergoline, deramciclane, amperozide, AMDA, cinanserin, LY-86057, GSK-215083, cyamemazine, mesulergine, BF-1, LY-215840, sergolexole, spiramide, LY-53857, amesergide, LY-108742, pipamperone, LY-314228, 5-I-R91150, 5-MeO-NBpBrT, 9-Aminomethyl-9,10-dihydroanthracene, niaprazine, SB-215505, SB-204741, SB-206553, SB-242084, LY-272015, SB-243213, SB-200646, RS-102221, zotepine, clozapine, chlorpromazine, sertindole, iloperidone, risperidone, paliperidone, asenapine, amisulpride, aripiprazole, brexpiprazole, lurasidone, ziprasidone, lumateperone, perospirone, mosapramine, adatanserin, AMDA (9-Aminomethyl-9,10-dihydroanthracene), cinanserin, fananserin, iferanserin, methiothepin, an extended-release form of olanzapine (e.g., ZYPREXA RELPREVV), an extended-release form of quetiapine, an extended-release form of risperidone (e.g., Risperdal Consta), an extended-release form of paliperidone (e.g., Invega Sustenna and Invega Trinza), an extended-release form of fluphenazine decanoate including Prolixin Decanoate, an extended-release form of aripiprazole lauroxil including Aristada, and an extended-release form of aripiprazole including Abilify Maintena, or a pharmaceutically acceptable salt, solvate, metabolite, deuterated analog, derivative, prodrug, or combinations thereof. In some embodiments, the serotonin receptor modulator for combination with the presently disclosed compounds is selected from MDL-11,939, eplivanserin (SR-46,349), ketanserin, ritanserin, altanserin, acepromazine, mianserin, mirtazapine, quetiapine, SB204741, SB206553, SB242084, LY272015, SB243213, blonanserin, SB200646, RS102221, nefazodone, MDL-100,907, pimavanserin, pruvanserin, nelotanserin and lorcaserin.

In certain embodiments the serotonin receptor modulator is selected from the group consisting of altanserin, blonanserin, eplivanserin, glemanserin, volinanserin, ketanserin, ritanserin, pimavanserin, nelotanserin, pruvanserin, and flibanserin. In one embodiment, the serotonin receptor modulator is selected from the group consisting of eplivanserin, volinanserin, ketanserin, ritanserin, pimavanserin, nelotanserin, pruvanserin, and flibanserin.

In some embodiments, the serotonin receptor modulator is ketanserin or a pharmaceutically acceptable salt, solvate, metabolite, deuterated analog, derivative, or prodrug thereof. In some embodiments, the serotonin receptor modulator is pimavanserin or a pharmaceutically acceptable salt, solvate, metabolite, deuterated analog, derivative, or prodrug thereof. In some embodiments, the serotonin receptor modulator is eplivanserin or a pharmaceutically acceptable salt, solvate, metabolite, deuterated analog, derivative, or prodrug thereof. In some embodiments, the serotonin receptor modulator is flibanserin or a pharmaceutically acceptable salt, solvate, metabolite, deuterated analog, derivative, or prodrug thereof. In some embodiments, the serotonin receptor modulator is roluperiodone or a pharmaceutically acceptable salt, solvate, metabolite, deuterated analog, derivative, or prodrug thereof.

In some embodiments, the serotonin receptor modulator is administered prior to a compound disclosed herein, such as about three hours prior, or from about one to about three hours prior to administration of a compound according to Formula I, I-1, I-1A, I-1B, II, IIa, IIb, IIc, IId, IIe, IIf, IIg, Table 1, or a pharmaceutically acceptable salt thereof. In some embodiments, the serotonin receptor modulator is administered at most about one hour prior to the presently disclosed compound. Thus, in some embodiments of combination therapy with the presently disclosed compounds, the second therapeutic agent is a serotonin receptor modulator. In some embodiments the second therapeutic agent serotonin receptor modulator is provided at a dose of from about 10 mg to about 350 mg. In some embodiments, the serotonin receptor modulator is provided at a dose of from about 20 mg to about 200 mg. In some embodiments, the serotonin receptor modulator is provided at a dose of from about 10 mg to about 100 mg. In certain such embodiments, the compound of the present invention is provided at a dose of from about 10 mg to about 100 mg, or from about 20 to about 200 mg, or from about 15 to about 300 mg, and the serotonin receptor modulator is provided at a dose of about 1 mg to about 100 mg.

In some embodiments, the serotonin receptor modulator for use with psychedelic azepino indole analogs disclosed herein, including those described in Table 1, is eplivanserin, wherein the eplivanserin is administered in about 1 mg to about 40 mg, or about 5 mg to about 10 mg and the azepino indole analog is present between about 1 mg and about 1,000 mg, or between about 500 mg and 800 mg.

In some embodiments, the serotonin receptor modulator for use with the psychedelic azepino indole analog disclosed herein, including those described in Table 1, is volinanserin, wherein the volinanserin is administered in about 1 mg to about 60 mg, or about 5 mg to about 20 mg and the azepino indole analog is present between about 1 mg and about 1,000 mg, or between about 500 mg and 800 mg.

In some embodiments, the serotonin receptor modulator for use with the psychedelic azepino indole analog disclosed herein, including those described in Table 1, is ketanserin, wherein the ketanserin is administered in about 10 mg to about 80 mg, about 30 mg to about 50 mg, or about 40 mg and the azepino indole analog is present between about 1 mg and about 1,000 mg, or between about 500 mg and 800 mg.

In some embodiments, the serotonin receptor modulator for use with the psychedelic azepino indole analog disclosed herein, including those described in Table 1, is ritanserin, wherein the ritanserin is administered in about 1 mg to about 40 mg, or about 2.5 mg to about 10 mg and the azepino indole analog is present between about 1 mg and about 1,000 mg, or between about 500 mg and 800 mg.

In some embodiments, the serotonin receptor modulator for use with the psychedelic azepino indole analog disclosed herein, including those described in Table 1, is pimavanserin, wherein the pimavanserin is administered in about 1 mg to about 60 mg, or about 17 mg to about 34 mg and the azepino indole analog is present between about 1 mg and about 1,000 mg, or between about 500 mg and 800 mg.

In some embodiments, the serotonin receptor modulator for use with the psychedelic azepino indole analog disclosed herein, including those described in Table 1, is nelotanserin, wherein the nelotanserin is administered in about 1 mg to about 80 mg, or about 40 mg to about 80 mg and the azepino indole analog is present between about 1 mg and about 1,000 mg, or between about 500 mg and 800 mg.

In some embodiments, the serotonin receptor modulator for use with the psychedelic azepino indole analog disclosed herein, including those described in Table 1, is pruvanserin, wherein the pruvanserin is administered in about 1 mg to about 40 mg, or about 3 mg to about 10 mg and the azepino indole analog is present between about 1 mg and about 1,000 mg, or between about 500 mg and 800 mg.

In some embodiments, the serotonin receptor modulator for use with the psychedelic azepino indole analog disclosed herein, including those described in Table 1, is flibanserin, wherein the flibanserin is administered in about 10 mg to about 200 mg, or about 80 mg to about 120 mg, or about 100 mg and the azepino indole analog is present between about 1 mg and about 1,000 mg, or between about 500 mg and 800 mg.

In certain embodiments, such as those described above a compound of Formula I, I-1, I-1A, I-1B, II, IIa, IIb, IIc, IId, IIe, Hf, IIg, Table 1, or a pharmaceutically acceptable salt thereof is co-administered with a serotonin receptor modulator in the same or in separate compositions. In one embodiment, the serotonin receptor modulator is administered prior to the compound of Formula I, I-1, I-1A, I-1B, II, IIa, IIb, IIc, IId, IIe, Hf, IIg or a pharmaceutically acceptable salt thereof. In one embodiment, the compound according to Formula I, I-1, I-1A, I-1B, II, IIa, IIb, IIc, IId, IIe, IIf, IIg, Table 1, or a pharmaceutically acceptable salt thereof is administered in a modified release formulation such that the subject is effectively pretreated with serotonin receptor modulator prior to release of an effective amount of the psychedelic. Thus, in some embodiments, the serotonin receptor modulator is administered or released from a composition provided herein prior to the administration and/or release of the psychedelic. This allows pretreatment to attenuate activation of the serotonin receptor by the psychedelic.

In some embodiments, the serotonin receptor modulator is administered or released from the composition provided herein to pretreat a subject by at least about at about 5 minutes, 10 minutes, 20 minutes, 30 minutes, 40 minutes, 50 minutes, 1 hour, 1.25 hours, 1.5 hours, 2 hours, or 3 hours prior to the release of the psychedelic azepino indole analog. In some embodiments, the serotonin receptor modulator attenuates the activation of the serotonin receptor when the serotonin receptor modulator is used to pretreat at most about 3 hours, 4 hours, 5 hours, 6 hours, 7 hours, 8 hours, 9 hours, or more than 9 hours prior to the release of the psychedelic. In some embodiments, the serotonin receptor modulator attenuates the activation of the serotonin receptor when the serotonin receptor modulator is used to pretreat in a range of about 5 minutes to about 3 hours, about 10 minutes to about 3 hours, about 20 minutes to about 3 hours, about 30 minutes to about 3 hours, about 40 minutes to about 3 hours, about 50 minutes to about 3 hours, about 1 hour to about 3 hours, about 5 minutes to about 2 hours, about 10 minutes to about 2 hours, about 20 minutes to about 2 hours, about 30 minutes to about 2 hours, about 40 minutes to about 2 hours, about 50 minutes to about 2 hours, about 1 hour to about 2 hours, about 5 minutes to about 1 hour, about 10 minutes to about 1 hour, about 20 minutes to about 1 hour, about 30 minutes to about 1 hour, about 40 minutes to about 1 hour, or about 50 minutes to about 1 hour prior to the release of the psychedelic.

In a preferred embodiment, the serotonin receptor modulator is administered at about 1 hour to about 3 hours prior to the administration of the psychedelic.

In some embodiments, the serotonin receptor modulator is eplivanserin, wherein the eplivanserin is administered to pretreat at least 15 minutes prior to the administration of the azepino indole analog disclosed herein, including those described in Table 1. In some embodiments, the serotonin receptor modulator is eplivanserin, wherein the eplivanserin is administered to pretreat between at least 30 minutes prior and 360 minutes prior to the release or administration of the azepino indole analog disclosed herein, including those described in Table 1. In some embodiments, the serotonin receptor modulator is eplivanserin, wherein the eplivanserin is administered to pretreat between at least 60 minutes prior and 360 minutes prior to the release or administration the azepino indole analog disclosed herein, including those described in Table 1. In some embodiments, the serotonin receptor modulator is eplivanserin, wherein the eplivanserin is administered to pretreat between at least 90 minutes and 240 minutes prior to the azepino indole analog disclosed herein, including those described in Table 1. In some embodiments, the serotonin receptor modulator is eplivanserin, wherein the eplivanserin is administered to pretreat at least 120 minutes prior to the azepino indole analog disclosed herein, including those described in Table 1.

In some embodiments, the serotonin receptor modulator is eplivanserin, wherein the eplivanserin is administered to pretreat at least 150 minutes prior to the azepino indole analog disclosed herein, including those described in Table 1. In some embodiments, the serotonin receptor modulator is eplivanserin, wherein the eplivanserin is administered to pretreat between about 15 minutes and 150 minutes prior to the azepino indole analog disclosed herein, including those described in Table 1. In some embodiments, the serotonin receptor modulator is eplivanserin, wherein the eplivanserin is administered to pretreat at least 180 minutes prior to the azepino indole analog disclosed herein, including those described in Table 1. In some embodiments, the serotonin receptor modulator is eplivanserin, wherein the eplivanserin is administered to pretreat at least 210 minutes prior to the azepino indole analog disclosed herein, including those described in Table 1. In some embodiments, the serotonin receptor modulator is eplivanserin, wherein the eplivanserin is administered to pretreat at least 240 minutes prior to the azepino indole analog disclosed herein, including those described in Table 1.

In some embodiments, the serotonin receptor modulator is eplivanserin, wherein the eplivanserin is administered to pretreat at least 270 minutes prior to the azepino indole analog disclosed herein, including those described in Table 1. In some embodiments, the serotonin receptor modulator is eplivanserin, wherein the eplivanserin is administered to pretreat at least 300 minutes prior to the azepino indole analog disclosed herein, including those described in Table 1. In some embodiments, the serotonin receptor modulator is eplivanserin, wherein the eplivanserin is administered to pretreat at least 330 minutes prior to the azepino indole analog disclosed herein, including those described in Table 1. In some embodiments, the serotonin receptor modulator is eplivanserin, wherein the eplivanserin is administered to pretreat at least 360 minutes prior to administration or release of the azepino indole analog disclosed herein, including those described in Table 1.

In some embodiments, the serotonin receptor modulator is eplivanserin, wherein eplivanserin is administered to pretreat between about 60 minutes and about 180 minutes prior to the administration or release of the azepino indole analog disclosed herein, including those described in Table 1.

In some embodiments, the serotonin receptor modulator is volinanserin, wherein the volinanserin is administered to pretreat a subject between at least 15 minutes and 360 minutes prior to the administration or release of the azepino indole analog disclosed herein, including those described in Table 1. In some embodiments, the serotonin receptor modulator is volinanserin, wherein the volinanserin is administered to pretreat between at least 30 minutes and 360 minutes prior to the administration or release of the azepino indole analog disclosed herein, including those described in Table 1. In some embodiments, the serotonin receptor modulator is volinanserin, wherein the volinanserin is administered to pretreat between at least 60 minutes and 240 minutes prior to the administration or release of the azepino indole analog disclosed herein, including those described in Table 1. In some embodiments, the serotonin receptor modulator is volinanserin, wherein the volinanserin is administered to pretreat at least 90 minutes prior to azepino indole analog disclosed herein, including those described in Table 1. In some embodiments, the serotonin receptor modulator is volinanserin, wherein the volinanserin is administered to pretreat at least 120 minutes prior to the azepino indole analog disclosed herein, including those described in Table 1.

In some embodiments, the serotonin receptor modulator is volinanserin, wherein the volinanserin is administered to pretreat at least 150 minutes prior to the azepino indole analog disclosed herein, including those described in Table 1. In some embodiments, the serotonin receptor modulator is volinanserin, wherein the volinanserin is administered to pretreat between about 15 minutes and about 150 minutes prior to the azepino indole analog disclosed herein, including those described in Table 1. In some embodiments, the serotonin receptor modulator is volinanserin, wherein the volinanserin is administered to pretreat at least 180 minutes prior to the azepino indole analog disclosed herein, including those described in Table 1. In some embodiments, the serotonin receptor modulator is volinanserin, wherein the volinanserin is administered to pretreat at least 210 minutes prior to the azepino indole analog disclosed herein, including those described in Table 1. In some embodiments, the serotonin receptor modulator is volinanserin, wherein the volinanserin is administered to pretreat at least 240 minutes prior to the azepino indole analog disclosed herein, including those described in Table 1. In some embodiments, the serotonin receptor modulator is volinanserin, wherein the volinanserin is administered to pretreat at least 270 minutes prior to the azepino indole analog disclosed herein, including those described in Table 1. In some embodiments, the serotonin receptor modulator is volinanserin, wherein the volinanserin is administered to pretreat at least 300 minutes prior to the azepino indole analog disclosed herein, including those described in Table 1. In some embodiments, the serotonin receptor modulator is volinanserin, wherein the volinanserin is administered to pretreat at least 330 minutes prior to azepino indole analog disclosed herein, including those described in Table 1. In some embodiments, the serotonin receptor modulator is volinanserin, wherein the volinanserin is administered to pretreat at least 360 minutes prior to the azepino indole analog disclosed herein, including those described in Table 1. In some embodiments, the serotonin receptor modulator is volinanserin, wherein volinanserin is administered to pretreat between about 60 minutes and about 180 minutes prior to the administration of the azepino indole analog disclosed herein, including those described in Table 1.

In some embodiments, the serotonin receptor modulator is ketanserin, wherein the ketanserin is administered to pretreat at least 15 minutes prior to the administration of the azepino indole analog disclosed herein, including those described in Table 1. In some embodiments, the serotonin receptor modulator is ketanserin, wherein the ketanserin is administered to pretreat between at least 30 minutes and 360 minutes prior to the administration or release of the azepino indole analog disclosed herein, including those described in Table 1. In some embodiments, the serotonin receptor modulator is ketanserin, wherein the ketanserin is administered to pretreat between at least 60 minutes and 240 minutes prior to the administration or release of the azepino indole analog disclosed herein, including those described in Table 1. In some embodiments, the serotonin receptor modulator is ketanserin, wherein the ketanserin is administered to pretreat at least 90 minutes prior to the azepino indole analog disclosed herein, including those described in Table 1. In some embodiments, the serotonin receptor modulator is ketanserin, wherein the ketanserin is administered to pretreat at least 120 minutes prior to the azepino indole analog disclosed herein, including those described in Table 1. In some embodiments, the serotonin receptor modulator is ketanserin, wherein the ketanserin is administered to pretreat at least 150 minutes prior to the azepino indole analog disclosed herein, including those described in Table 1. In some embodiments, the serotonin receptor modulator is ketanserin, wherein the ketanserin is administered to pretreat between about 15 minutes and about 150 minutes prior to the azepino indole analog disclosed herein, including those described in Table 1. In some embodiments, the serotonin receptor modulator is ketanserin, wherein the ketanserin is administered to pretreat at least 180 minutes prior to the azepino indole analog disclosed herein, including those described in Table 1. In some embodiments, the serotonin receptor modulator is ketanserin, wherein the ketanserin is administered to pretreat at least 210 minutes prior to the azepino indole analog disclosed herein, including those described in Table 1. In some embodiments, the serotonin receptor modulator is ketanserin, wherein the ketanserin is administered to pretreat at least 240 minutes prior to the azepino indole analog disclosed herein, including those described in Table 1. In some embodiments, the serotonin receptor modulator is ketanserin, wherein the ketanserin is administered to pretreat at least 270 minutes prior to the azepino indole analog disclosed herein, including those described in Table 1. In some embodiments, the serotonin receptor modulator is ketanserin, wherein the ketanserin is administered to pretreat at least 300 minutes prior to the azepino indole analog disclosed herein, including those described in Table 1. In some embodiments, the serotonin receptor modulator is ketanserin, wherein the ketanserin is administered to pretreat at least 330 minutes prior to the azepino indole analog disclosed herein, including those described in Table 1. In some embodiments, the serotonin receptor modulator is ketanserin, wherein the ketanserin is administered to pretreat at least 360 minutes prior to the azepino indole analog disclosed herein, including those described in Table 1. In some embodiments, the serotonin receptor modulator is ketanserin, wherein ketanserin is administered to pretreat between about 60 minutes and about 180 minutes prior to the administration of the azepino indole analog disclosed herein, including those described in Table 1.

In some embodiments, the serotonin receptor modulator is ritanserin, wherein the ritanserin is administered to pretreat at least 15 minutes prior to the administration of the azepino indole analog disclosed herein, including those described in Table 1. In some embodiments, the serotonin receptor modulator is ritanserin, wherein the ritanserin is administered to pretreat at least 30 minutes prior to the azepino indole analog disclosed herein, including those described in Table 1. In some embodiments, the serotonin receptor modulator is ritanserin wherein the ritanserin is administered to pretreat between at least 60 minutes and 240 minutes prior to the administration or release of the azepino indole analog disclosed herein, including those described in Table 1. In some embodiments, the serotonin receptor modulator is ritanserin, wherein the ritanserin is administered to pretreat at least 90 minutes prior to the azepino indole analog disclosed herein, including those described in Table 1. In some embodiments, the serotonin receptor modulator is ritanserin, wherein the ritanserin is administered to pretreat at least 120 minutes prior to the azepino indole analog disclosed herein, including those described in Table 1. In some embodiments, the serotonin receptor modulator is ritanserin, wherein the ritanserin is administered to pretreat at least 150 minutes prior to the azepino indole analog disclosed herein, including those described in Table 1. In some embodiments, the serotonin receptor modulator is ritanserin, wherein the ritanserin is administered to pretreat between about 15 minutes and about 150 minutes prior to the azepino indole analog disclosed herein, including those described in Table 1. In some embodiments, the serotonin receptor modulator is ritanserin, wherein the ritanserin is administered to pretreat at least 180 minutes prior to the azepino indole analog disclosed herein, including those described in Table 1. In some embodiments, the serotonin receptor modulator is ritanserin, wherein the ritanserin is administered to pretreat at least 210 minutes prior to the azepino indole analog disclosed herein, including those described in Table 1. In some embodiments, the serotonin receptor modulator is ritanserin, wherein the ritanserin is administered to pretreat at least 240 minutes prior to the azepino indole analog disclosed herein, including those described in Table 1. In some embodiments, the serotonin receptor modulator is ritanserin, wherein the ritanserin is administered to pretreat at least 270 minutes prior to the azepino indole analog disclosed herein, including those described in Table 1. In some embodiments, the serotonin receptor modulator is ritanserin, wherein the ritanserin is administered to pretreat at least 300 minutes prior to the azepino indole analog disclosed herein, including those described in Table 1. In some embodiments, the serotonin receptor modulator is ritanserin, wherein the ritanserin is administered to pretreat at least 330 minutes prior to the azepino indole analog disclosed herein, including those described in Table 1. In some embodiments, the serotonin receptor modulator is ritanserin, wherein the ritanserin is administered to pretreat at least 360 minutes prior to the azepino indole analog disclosed herein, including those described in Table 1. In some embodiments, the serotonin receptor modulator is ritanserin, wherein ritanserin is administered to pretreat between about 60 minutes and about 180 minutes prior to the administration of the azepino indole analog disclosed herein, including those described in Table 1.

In some embodiments, the serotonin receptor modulator is pimavanserin, wherein the pimavanserin is administered to pretreat at least 15 minutes prior to the administration of the azepino indole analog disclosed herein, including those described in Table 1. In some embodiments, the serotonin receptor modulator is pimavanserin, wherein the pimavanserin is administered to pretreat at least 30 minutes prior to the azepino indole analog disclosed herein, including those described in Table 1. In some embodiments, the serotonin receptor modulator is pimavanserin, wherein the pimavanserin is administered to pretreat between at least 60 minutes and 240 minutes prior to the administration or release of the azepino indole analog disclosed herein, including those described in Table 1. In some embodiments, the serotonin receptor modulator is pimavanserin, wherein the pimavanserin is administered to pretreat at least 90 minutes prior to the azepino indole analog disclosed herein, including those described in Table 1. In some embodiments, the serotonin receptor modulator is pimavanserin, wherein the pimavanserin is administered to pretreat at least 120 minutes prior to the azepino indole analog disclosed herein, including those described in Table 1. In some embodiments, the serotonin receptor modulator is pimavanserin, wherein the pimavanserin is administered to pretreat at least 150 minutes prior to the azepino indole analog disclosed herein, including those described in Table 1. In some embodiments, the serotonin receptor modulator is pimavanserin, wherein the pimavanserin is administered to pretreat between about 15 minutes and about 150 minutes prior to the azepino indole analog disclosed herein, including those described in Table 1. In some embodiments, the serotonin receptor modulator is pimavanserin, wherein the pimavanserin is administered to pretreat at least 180 minutes prior to the azepino indole analog disclosed herein, including those described in Table 1. In some embodiments, the serotonin receptor modulator is pimavanserin, wherein the pimavanserin is administered to pretreat at least 210 minutes prior to the azepino indole analog disclosed herein, including those described in Table 1. In some embodiments, the serotonin receptor modulator is pimavanserin, wherein the pimavanserin is administered to pretreat at least 240 minutes prior to the azepino indole analog disclosed herein, including those described in Table 1. In some embodiments, the serotonin receptor modulator is pimavanserin, wherein the pimavanserin is administered to pretreat at least 270 minutes prior to the azepino indole analog disclosed herein, including those described in Table 1. In some embodiments, the serotonin receptor modulator is pimavanserin, wherein the pimavanserin is administered to pretreat at least 300 minutes prior to the azepino indole analog disclosed herein, including those described in Table 1. In some embodiments, the serotonin receptor modulator is pimavanserin, wherein the pimavanserin is administered to pretreat at least 330 minutes prior to the azepino indole analog disclosed herein, including those described in Table 1. In some embodiments, the serotonin receptor modulator is pimavanserin, wherein the pimavanserin is administered to pretreat at least 360 minutes prior to the azepino indole analog disclosed herein, including those described in Table 1. In some embodiments, the serotonin receptor modulator is pimavanserin, wherein pimavanserin is administered to pretreat between about 60 minutes and about 180 minutes prior to the administration of the azepino indole analog disclosed herein, including those described in Table 1.

In some embodiments, the serotonin receptor modulator is nelotanserin, wherein the nelotanserin is administered to pretreat at least 15 minutes prior to the administration of the azepino indole analog disclosed herein, including those described in Table 1. In some embodiments, the serotonin receptor modulator is nelotanserin, wherein the nelotanserin is administered to pretreat at least 30 minutes prior to the azepino indole analog disclosed herein, including those described in Table 1. In some embodiments, the serotonin receptor modulator is nelotanserin, wherein the nelotanserin is administered to pretreat between at least 60 minutes and 240 minutes prior to the administration or release of the azepino indole analog disclosed herein, including those described in Table 1. In some embodiments, the serotonin receptor modulator is nelotanserin, wherein the nelotanserin is administered to pretreat at least 90 minutes prior to the azepino indole analog disclosed herein, including those described in Table 1. In some embodiments, the serotonin receptor modulator is nelotanserin, wherein the nelotanserin is administered to pretreat at least 120 minutes prior to the azepino indole analog disclosed herein, including those described in Table 1.

In some embodiments, the serotonin receptor modulator is nelotanserin, wherein the nelotanserin is administered to pretreat at least 150 minutes prior to the azepino indole analog disclosed herein, including those described in Table 1. In some embodiments, the serotonin receptor modulator is nelotanserin, wherein the nelotanserin is administered to pretreat between about 15 minutes and about 150 minutes prior to the azepino indole analog disclosed herein, including those described in Table 1. In some embodiments, the serotonin receptor modulator is nelotanserin, wherein the nelotanserin is administered to pretreat at least 180 minutes prior to the azepino indole analog disclosed herein, including those described in Table 1. In some embodiments, the serotonin receptor modulator is nelotanserin, wherein the nelotanserin is administered to pretreat at least 210 minutes prior to the azepino indole analog disclosed herein, including those described in Table 1. In some embodiments, the serotonin receptor modulator is nelotanserin, wherein the nelotanserin is administered to pretreat at least 240 minutes prior to the azepino indole analog disclosed herein, including those described in Table 1. In some embodiments, the serotonin receptor modulator is nelotanserin, wherein the nelotanserin is administered to pretreat at least 270 minutes prior to the azepino indole analog disclosed herein, including those described in Table 1.

In some embodiments, the serotonin receptor modulator is nelotanserin, wherein the nelotanserin is administered to pretreat at least 300 minutes prior to the azepino indole analog disclosed herein, including those described in Table 1. In some embodiments, the serotonin receptor modulator is nelotanserin, wherein the nelotanserin is administered to pretreat at least 330 minutes prior to the azepino indole analog disclosed herein, including those described in Table 1. In some embodiments, the serotonin receptor modulator is nelotanserin, wherein the nelotanserin is administered to pretreat at least 360 minutes prior to the azepino indole analog disclosed herein, including those described in Table 1. In some embodiments, the serotonin receptor modulator is nelotanserin, wherein nelotanserin is administered to pretreat between about 60 minutes and about 180 minutes prior to the administration of the azepino indole analog disclosed herein, including those described in Table 1.

In some embodiments, the serotonin receptor modulator is pruvanserin, wherein the pruvanserin is administered to pretreat at least 15 minutes prior to the administration of the azepino indole analog disclosed herein, including those described in Table 1. In some embodiments, the serotonin receptor modulator is pruvanserin, wherein the pruvanserin is administered to pretreat at least 30 minutes prior to the azepino indole analog disclosed herein, including those described in Table 1. In some embodiments, the serotonin receptor modulator is pruvanserin, wherein the pruvanserin is administered to pretreat between at least 60 minutes and 240 minutes prior to the administration or release of the azepino indole analog disclosed herein, including those described in Table 1. In some embodiments, the serotonin receptor modulator is pruvanserin, wherein the pruvanserin is administered to pretreat at least 90 minutes prior to the azepino indole analog disclosed herein, including those described in Table 1. In some embodiments, the serotonin receptor modulator is pruvanserin, wherein the pruvanserin is administered to pretreat at least 120 minutes prior to the azepino indole analog disclosed herein, including those described in Table 1. In some embodiments, the serotonin receptor modulator is pruvanserin, wherein the pruvanserin is administered to pretreat at least 150 minutes prior to the azepino indole analog disclosed herein, including those described in Table 1. In some embodiments, the serotonin receptor modulator is pruvanserin, wherein the pruvanserin is administered to pretreat between about 15 minutes and about 150 minutes prior to the azepino indole analog disclosed herein, including those described in Table 1. In some embodiments, the serotonin receptor modulator is pruvanserin, wherein the pruvanserin is administered to pretreat at least 180 minutes prior to the azepino indole analog disclosed herein, including those described in Table 1. In some embodiments, the serotonin receptor modulator is pruvanserin, wherein the pruvanserin is administered to pretreat at least 210 minutes prior to the azepino indole analog disclosed herein, including those described in Table 1.

In some embodiments, the serotonin receptor modulator is pruvanserin, wherein the pruvanserin is administered to pretreat at least 240 minutes prior to the azepino indole analog disclosed herein, including those described in Table 1. In some embodiments, the serotonin receptor modulator is pruvanserin, wherein the pruvanserin is administered to pretreat at least 270 minutes prior to the azepino indole analog disclosed herein, including those described in Table 1. In some embodiments, the serotonin receptor modulator is pruvanserin, wherein the pruvanserin is administered to pretreat at least 300 minutes prior to the azepino indole analog disclosed herein, including those described in Table 1. In some embodiments, the serotonin receptor modulator is pruvanserin, wherein the pruvanserin is administered to pretreat at least 330 minutes prior to the azepino indole analog disclosed herein, including those described in Table 1. In some embodiments, the serotonin receptor modulator is pruvanserin, wherein the pruvanserin is administered to pretreat at least 360 minutes prior to the azepino indole analog disclosed herein, including those described in Table 1. In some embodiments, the serotonin receptor modulator is pruvanserin, wherein pruvanserin is administered to pretreat between about 60 minutes and about 180 minutes prior to the administration of the azepino indole analog disclosed herein, including those described in Table 1.

In some embodiments, the serotonin receptor modulator is flibanserin, wherein the flibanserin is administered to pretreat at least 15 minutes prior to the administration of the azepino indole analog disclosed herein, including those described in Table 1. In some embodiments, the serotonin receptor modulator is flibanserin, wherein the flibanserin is administered to pretreat at least 30 minutes prior to the azepino indole analog disclosed herein, including those described in Table 1. In some embodiments, the serotonin receptor modulator is flibanserin, wherein the flibanserin is administered to pretreat between at least 60 minutes and 240 minutes prior to the administration or release of the azepino indole analog disclosed herein, including those described in Table 1. In some embodiments, the serotonin receptor modulator is flibanserin, wherein the flibanserin is administered to pretreat at least 90 minutes prior to the azepino indole analog disclosed herein, including those described in Table 1. In some embodiments, the serotonin receptor modulator is flibanserin, wherein the flibanserin is administered to pretreat at least 120 minutes prior to the azepino indole analog disclosed herein, including those described in Table 1. In some embodiments, the serotonin receptor modulator is flibanserin, wherein the flibanserin is administered to pretreat at least 150 minutes prior to the azepino indole analog disclosed herein, including those described in Table 1. In some embodiments, the serotonin receptor modulator is flibanserin, wherein the flibanserin is administered to pretreat between about 15 minutes and about 150 minutes prior to the azepino indole analog disclosed herein, including those described in Table 1. In some embodiments, the serotonin receptor modulator is flibanserin, wherein the flibanserin is administered to pretreat at least 180 minutes prior to the azepino indole analog disclosed herein, including those described in Table 1. In some embodiments, the serotonin receptor modulator is flibanserin, wherein the flibanserin is administered to pretreat at least 210 minutes prior to the azepino indole analog disclosed herein, including those described in Table 1.

In some embodiments, the serotonin receptor modulator is flibanserin, wherein the flibanserin is administered to pretreat at least 240 minutes prior to the azepino indole analog disclosed herein, including those described in Table 1. In some embodiments, the serotonin receptor modulator is flibanserin, wherein the flibanserin is administered to pretreat at least 270 minutes prior to the azepino indole analog disclosed herein, including those described in Table 1. In some embodiments, the serotonin receptor modulator is flibanserin, wherein the flibanserin is administered to pretreat at least 300 minutes prior to the azepino indole analog disclosed herein, including those described in Table 1. In some embodiments, the serotonin receptor modulator is flibanserin, wherein the flibanserin is administered to pretreat at least 330 minutes prior to the azepino indole analog disclosed herein, including those described in Table 1. In some embodiments, the serotonin receptor modulator is flibanserin, wherein the flibanserin is administered to pretreat at least 360 minutes prior to the azepino indole analog disclosed herein, including those described in Table 1. In some embodiments, the serotonin receptor modulator is flibanserin, wherein flibanserin is administered to pretreat between about 60 minutes and about 180 minutes prior to the administration of the azepino indole analog disclosed herein, including those described in Table 1.

In some embodiments, the compounds disclosed herein are non-hallucinogenic 5-HT_{2A} modulators (e.g., 5-HT_{2A} agonists) that are used to treat neurological diseases. In some embodiments, the neurological diseases comprise decreased neural plasticity, decreased cortical structural plasticity, decreased 5-HT_{2A} receptor content, decreased dendritic arbor complexity, loss of dendritic spines, decreased dendritic branch content, decreased spinogenesis, decreased neuritogenesis, retraction of neurites, or any combination thereof.

In some embodiments, the present compounds are non-hallucinogenic 5-HT_{2A} modulators (e.g., 5-HT_{2A} agonists) that are used for increasing neuronal plasticity. In some embodiments, the present compounds are non-hallucinogenic 5-HT_{2A} modulators (e.g., 5-HT_{2A} agonists) that are used for treating a brain disorder. In some embodiments, the present compounds are non-hallucinogenic 5-HT_{2A} modulators (e.g., 5-HT_{2A} agonists) are used for increasing at least one of translation, transcription, or secretion of neurotrophic factors.

### Methods for Increasing Neuronal Plasticity

Neuronal plasticity refers to the ability of the brain to change structure and/or function throughout a subject's life. New neurons can be produced and integrated into the central nervous system throughout the subject's life. Increasing neuronal plasticity includes, but is not limited to, promoting neuronal growth, promoting neuritogenesis, promoting synaptogenesis, promoting dendritogenesis, increasing dendritic arbor complexity, increasing dendritic spine density, and increasing excitatory synapsis in the brain. In some embodiments, increasing neuronal plasticity comprises promoting neuronal growth, promoting neuritogenesis, promoting synaptogenesis, promoting dendritogenesis, increasing dendritic arbor complexity, and increasing dendritic spine density.

In some embodiments, increasing neuronal plasticity by treating a subject with a compound of Formula I, I-1, I-1A, I-1B, II, IIa, IIb, IIc, IId, IIe, IIf, IIg, Table 1, or a pharmaceutically acceptable salt thereof can treat neurodegenerative disorder, Alzheimer's, Parkinson's disease, psychological disorder, depression, addiction, anxiety, post-traumatic stress disorder, treatment resistant depression, suicidal ideation, major depressive disorder, bipolar disorder, schizophrenia, stroke, traumatic brain injury, or substance use disorder.

In some embodiments, the present disclosure provides methods for increasing neuronal plasticity, comprising contacting a neuronal cell with a compound of the present invention, such as a compound of Formula I, I-1, I-1A, I-1B, II, IIa, IIb, IIc, IId, IIe, IIf, IIg, Table 1, or a pharmaceutically acceptable salt thereof. In some embodiments, increasing neuronal plasticity improves a brain disorder described herein.

In some embodiments, a compound of the present disclosure is used to increase neuronal plasticity. In some embodiments, the compounds used to increase neuronal plasticity have, for example, anti-addictive properties, antidepressant properties, anxiolytic properties, or a combination thereof. In some embodiments, decreased neuronal plasticity is associated with a neuropsychiatric disease. In some embodiments, the neuropsychiatric disease is a mood or anxiety disorder. In some embodiments, the neuropsychiatric disease includes, for example, migraine, cluster headache, post-traumatic stress disorder (PTSD), schizophrenia, anxiety, depression, and addiction (e.g., substance abuse disorder). In some embodiments, brain disorders include, for example, migraines, addiction (e.g., substance use disorder), depression, and anxiety.

In some embodiments, the experiment or assay to determine increased neuronal plasticity of any compound of the present invention is a phenotypic assay, a dendritogenesis assay, a spinogenesis assay, a synaptogenesis assay, a Sholl analysis, a concentration-response experiment, a 5-HT_{2A} agonist assay, a 5-HT_{2A} antagonist assay, a 5-HT_{2A} binding assay, or a 5-HT_{2A} blocking experiment (e.g., ketanserin blocking experiments). In some embodiments, the experiment or assay to determine the hallucinogenic potential of any compound of the present invention is a mouse head-twitch response (HTR) assay.

In some embodiments, the present invention provides a method for increasing neuronal plasticity, comprising contacting a neuronal cell with a compound of Formula I, I-1, I-1A, I-1B, II, IIa, IIb, IIc, IId, IIe, IIf, IIg, Table 1, or a pharmaceutically acceptable salt thereof.

### Methods of Treating a Brain Disorder

In some embodiments, the present invention provides a method of treating a disease, including administering to a subject in need thereof, a therapeutically effective amount of a compound of Formula I, I-1, I-1A, I-1B, II, IIa, IIb, IIc, IId, IIe, IIf, IIg, Table 1, or a pharmaceutically acceptable salt thereof. In some embodiments, the present invention provides a method of treating a disease, including administering to a subject in need thereof, a therapeutically effective amount of azepino indole compound disclosed herein, including those described in Table 1, and representative compounds of the application, including Compound 1, Compound 2, Compound 163, Compound 140, Compound 144, Compound 143, Compound 164, Compound 146, Compound 138, Compound 110, Compound 109, Compound 165, Compound 117, Compound 145, Compound 124, Compound 166, Compound 122, and Compound 136. In some embodiments, the disease is a musculoskeletal pain disorder, such as fibromyalgia, muscle pain, joint stiffness, osteoarthritis, rheumatoid arthritis, or muscle cramps. In some embodiments, the present disclosure provides a method of treating a disease of women's reproductive health, such as premenstrual dysphoric disorder (PMDD), premenstrual syndrome (PMS), post-partum depression, or menopause. In some embodiments, the present disclosure provides a method of treating a brain disorder, including administering to a subject in need thereof, a therapeutically effective amount of a compound disclosed herein, such as a compound of Formula I, I-1, I-1A, I-1B, II, IIa, IIb, IIc, IId, IIe, IIf, IIg, Table 1, or a pharmaceutically acceptable salt thereof.

Diseases of particular interest include depression and related conditions. Accordingly, in some embodiments, the disease or disorder treated herein is depression or a disease or disorder related to depression. In some embodiments, the depression is major depressive disorder, persistent depressive disorder, bipolar disorder, treatment resistant depression (TRD), postpartum depression, premenstrual dysphoric disorder, or seasonal affective disorder. In some embodiments, the disease or disorder related to depression is anxiety. In some embodiments, methods of treating depression or a disease or disorder related to depression comprise treating the symptoms associated with the depression or the disease or disorder related to depression. Described herein are methods of treating depression or a disease or disorder related to depression in a subject in need thereof, the method comprising administering to the subject a disclosed compound. In some embodiments, the depression is major depressive disorder, persistent depressive disorder, bipolar disorder, treatment resistant depression (TRD), postpartum depression, premenstrual dysphoric disorder, or seasonal affective disorder. In some embodiments, the disease or disorder related to depression is anxiety. In some embodiments, methods of treating depression or a disease or disorder related to depression comprise treating the symptoms associated with the depression or the disease or disorder related to depression.

In some embodiments, the amount of compound in the composition is an amount effective to treat the relevant disease, disorder, or condition in a patient in need thereof (an “effective amount”). In some embodiments, a composition of the present disclosure is formulated for oral administration to a subject in need thereof.

In some embodiments, the present disclosure provides a method of treating a brain disorder with combination therapy, including administering to a subject in need thereof, a therapeutically effective amount of a compound disclosed herein and at least one additional therapeutic agent.

In some embodiments, serotonin receptor modulators, such as modulators of serotonin receptor 2A (5-HT_{2A} modulators, e.g., 5-HT_{2A} agonists), are used to treat a brain disorder. The presently disclosed compounds of Formulas I, I-1, I-1A, I-1B, II, IIa, IIb, IIc, IId, IIe, IIf, IIg, Table 1, or a pharmaceutically acceptable salts thereof can function as 5-HT_{2A} agonists alone, or in combination with a second therapeutic agent that also is a 5-HT_{2A} modulator. In such cases the second therapeutic agent can be an agonist or an antagonist. In some instances, it may be helpful administer a 5-HT_{2A} antagonist in combination with a compound of the present invention to mitigate undesirable effects of 5-HT_{2A} agonism, such as potential hallucinogenic effects. Serotonin receptor modulators useful as second therapeutic agents for combination therapy as described herein are known to those of skill in the art and include, without limitation, MDL-11,939, eplivanserin (SR-46,349), ketanserin, ritanserin, altanserin, acepromazine, mianserin, mirtazapine, quetiapine, SB204741, SB206553, SB242084, LY272015, SB243213, blonanserin, SB200646, RS102221, nefazodone, MDL-100,907, pimavanserin, nelotanserin and lorcaserin. In some embodiments, the serotonin receptor modulator used as a second therapeutic is pimavanserin or a pharmaceutically acceptable salt, solvate, metabolite, derivative, or prodrug thereof. In some embodiments, the serotonin receptor modulator is administered prior to a compound disclosed herein, such as about three or about hours prior administration of a compound according to Formula I, I-1, I-1A, I-1B, II, IIa, IIb, IIc, IId, IIe, IIf, IIg, Table 1, or a pharmaceutically acceptable salt thereof. In some embodiments, the serotonin receptor modulator is administered at most about one hour prior to the presently disclosed compound. Thus, in some embodiments of combination therapy with the presently disclosed compounds, the second therapeutic agent is a serotonin receptor modulator. In some embodiments the second therapeutic agent serotonin receptor modulator is provided at a dose of from about 10 mg to about 350 mg. In some embodiments, the serotonin receptor modulator is provided at a dose of from about 20 mg to about 200 mg. In some embodiments, the serotonin receptor modulator is provided at a dose of from about 10 mg to about 100 mg. In certain such embodiments, the compound of the present invention is provided at a dose of from about 10 mg to about 100 mg, or from about 20 to about 200 mg, or from about 15 to about 300 mg, and the serotonin receptor modulator is provided at a dose of about 10 mg to about 100 mg.

In some embodiments, 5-HT_{2A} modulators (e.g., 5-HT_{2A} agonists) are used to treat a brain disorder. In some embodiments, the brain disorders that can be treated as disclosed herein comprise decreased neural plasticity, decreased cortical structural plasticity, decreased 5-HT_{2A} receptor content, decreased dendritic arbor complexity, loss of dendritic spines, decreased dendritic branch content, decreased spinogenesis, decreased neuritogenesis, retraction of neurites, or any combination thereof.

In some embodiments, a compound disclosed herein, such as a compound of Formula I, I-1, I-1A, I-1B, II, IIa, IIb, IIc, IId, IIe, IIf, IIg, Table 1, or a pharmaceutically acceptable salt thereof is used to treat brain disorders. In some embodiments, the compounds have, for example, anti-addictive properties, antidepressant properties, anxiolytic properties, or a combination thereof. In some embodiments, the brain disorder is a neuropsychiatric disease. In some embodiments, the neuropsychiatric disease is a mood or anxiety disorder. In some embodiments, brain disorders include, for example, migraine, cluster headache, post-traumatic stress disorder (PTSD), anxiety, depression, schizophrenia, and addiction (e.g., substance abuse disorder). In some embodiments, brain disorders include, for example, migraines, addiction (e.g., substance use disorder), depression, and anxiety.

In some embodiments, the present invention provides a method of treating a brain disorder, comprising administering to a subject in need thereof a therapeutically effective amount of a compound disclosed herein, such as a compound of Formulas I, I-1, I-1A, I-1B, II, IIa, IIb, IIc, IId, IIe, IIf, IIg, Table 1, or a pharmaceutically acceptable salt thereof.

In some embodiments, the brain disorder is a neurodegenerative disorder, Alzheimer's disease, Parkinson's disease, psychological disorder, depression, addiction, anxiety, post-traumatic stress disorder, treatment resistant depression, postpartum depression, premenstrual dysphoric disorder, seasonal affective disorder, suicidal ideation, major depressive disorder, bipolar disorder, schizophrenia, stroke, traumatic brain injury, or substance use disorder.

In some embodiments, the brain disorder is a neurodegenerative disorder, Alzheimer's, or Parkinson's disease. In some embodiments, the brain disorder is a psychological disorder, depression, addiction, anxiety, or a post-traumatic stress disorder. In some embodiments, the brain disorder is depression. In some embodiments, the brain disorder is addiction. In some embodiments, the brain disorder is treatment resistant depression, suicidal ideation, major depressive disorder, bipolar disorder, schizophrenia, stroke, traumatic brain injury or substance use disorder. In some embodiments, the brain disorder is treatment resistant depression, suicidal ideation, major depressive disorder, persistent depressive disorder, bipolar disorder, schizophrenia, or substance use disorder. In some embodiments, the brain disorder is stroke or traumatic brain injury. In some embodiments, the brain disorder is treatment resistant depression, suicidal ideation, major depressive disorder, bipolar disorder, or substance use disorder. In some embodiments, the brain disorder is schizophrenia. In some embodiments, the brain disorder is alcohol use disorder.

In some embodiments, the method further comprises administering one or more additional therapeutic agent that is lithium, olanzapine (Zyprexa), quetiapine (Seroquel), risperidone (Risperdal), ariprazole (Abilify), ziprasidone (Geodon), clozapine (Clozaril), divalproex sodium (Depakote), lamotrigine (Lamictal), valproic acid (Depakene), carbamazepine (Equetro), topiramate (Topamax), levomilnacipran (Fetzima), duloxetine (Cymbalta, Yentreve), venlafaxine (Effexor), citalopram (Celexa), fluvoxamine (Luvox), escitalopram (Lexapro), fluoxetine (Prozac), paroxetine (Paxil), sertraline (Zoloft), clomipramine (Anafranil), amitriptyline (Elavil), desipramine (Norpramin), imipramine (Tofranil), nortriptyline (Pamelor), phenelzine (Nardil), tranylcypromine (Parnate), diazepam (Valium), alprazolam (Xanax), or clonazepam (Klonopin).

In certain embodiments of the method for treating a brain disorder disclosed herein with a compound according to Formulas I, I-1, I-1A, I-1B, II, IIa, IIb, IIc, IId, IIe, IIf, IIg, Table 1, or a pharmaceutically acceptable salt thereof, a second therapeutic agent that is an empathogenic agent is administered. Examples of suitable empathogenic agents for use in combination with a compound according to Formulas I, I-1, I-1A, I-1B, II, IIa, IIb, IIc, IId, IIe, IIf, IIg, Table 1, are selected from the phenethylamines, such as 3,4-methylenedioxymethamphetamine (MDMA) and analogs thereof. Other suitable empathogenic agents for use in combination with the presently disclosed compounds include, without limitation, N-Allyl-3,4-methylenedioxy-amphetamine (MDAL) N-Butyl-3,4-methylenedioxyamphetamine (MDBU) N-Benzyl-3,4-methylenedioxyamphetamine (MDBZ) N-Cyclopropylmethyl-3,4-methylenedioxyamphetamine (MDCPM) N,N-Dimethyl-3,4-methylenedioxyamphetamine (MDDM) N-Ethyl-3,4-methylenedioxyamphetamine (MDE; MDEA) N-(2-Hydroxyethyl)-3,4-methylenedioxy amphetamine (MDHOET) N-Isopropyl-3,4-methylenedioxyamphetamine (MDIP) N-Methyl-3,4-ethylenedioxyamphetamine (MDMC) N-Methoxy-3,4-methylenedioxyamphetamine (MDMEO) N-(2-Methoxyethyl)-3,4-methylenedioxyamphetamine (MDMEOET) alpha,alpha,N-Trimethyl-3,4-methylenedioxyphenethylamine (MDMP; 3,4-Methylenedioxy-N-methylphentermine) N-Hydroxy-3,4-methylenedioxyamphetamine (MDOH) 3,4-Methylenedioxyphenethylamine (MDPEA) alpha,alpha-Dimethyl-3,4-methylenedioxyphenethylamine (MDPH; 3,4-methylenedioxyphentermine) N-Propargyl-3,4-methylenedioxyamphetamine (MDPL) Methylenedioxy-2-aminoindane (MDAI) 1,3-Benzodioxolyl-N-methylbutanamine MBDB 3,4-methylenedioxy-N-methyl-α-ethylphenylethylamine 3,4-Methylenedioxyamphetamine (MDA) Methylone (also known as 3,4-methylenedioxy-N-methylcathinone) Ethylone (also known as 3,4-methylenedioxy-N-ethylcathinone) GHB or Gamma Hydroxybutyrate or sodium oxybate N-Propyl-3,4-methylenedioxyamphetamine (MDPR), and the like.

In some embodiments, the compounds of the present invention are used in combination with the standard of care therapy for a neurological disease described herein. Non-limiting examples of the standard of care therapies, may include, for example, lithium, olanzapine, quetiapine, risperidone, ariprazole, ziprasidone, clozapine, divalproex sodium, lamotrigine, valproic acid, carbamazepine, topiramate, levomilnacipran, duloxetine, venlafaxine, citalopram, fluvoxamine, escitalopram, fluoxetine, paroxetine, sertraline, clomipramine, amitriptyline, desipramine, imipramine, nortriptyline, phenelzine, tranylcypromine, diazepam, alprazolam, clonazepam, or any combination thereof. Nonlimiting examples of standard of care therapy for depression are sertraline, fluoxetine, escitalopram, venlafaxine, or aripiprazole. Non-limiting examples of standard of care therapy for depression are citralopram, escitalopram, fluoxetine, paroxetine, diazepam, or sertraline. Additional examples of standard of care therapeutics are known to those of ordinary skill in the art.

### Methods of Increasing at Least One of Translation, Transcription, or Secretion of Neurotrophic Factors

Neurotrophic factors refer to a family of soluble peptides or proteins which support the survival, growth, and differentiation of developing and mature neurons. Increasing at least one of translation, transcription, or secretion of neurotrophic factors can be useful for, but not limited to, increasing neuronal plasticity, promoting neuronal growth, promoting neuritogenesis, promoting synaptogenesis, promoting dendritogenesis, increasing dendritic arbor complexity, increasing dendritic spine density, and increasing excitatory synapsis in the brain. In some embodiments, increasing at least one of translation, transcription, or secretion of neurotrophic factors can increasing neuronal plasticity. In some embodiments, increasing at least one of translation, transcription, or secretion of neurotrophic factors can promoting neuronal growth, promoting neuritogenesis, promoting synaptogenesis, promoting dendritogenesis, increasing dendritic arbor complexity, and/or increasing dendritic spine density.

In some embodiments, 5-HT_{2A} modulators (e.g., 5-HT_{2A} agonists) are used to increase at least one of translation, transcription, or secretion of neurotrophic factors. In some embodiments, a compound of the present invention, such as a compound of Formula I, I-1, I-1A, I-1B, II, IIa, IIb, IIc, IId, IIe, IIf, IIg, Table 1, or a pharmaceutically acceptable salt thereof is used to increase at least one of translation, transcription, or secretion of neurotrophic factors. In some embodiments, increasing at least one of translation, transcription or secretion of neurotrophic factors treats a migraine, headaches (e.g., cluster headache), post-traumatic stress disorder (PTSD), anxiety, depression, neurodegenerative disorder, Alzheimer's disease, Parkinson's disease, psychological disorder, treatment resistant depression, suicidal ideation, major depressive disorder, bipolar disorder, schizophrenia, stroke, traumatic brain injury, and addiction (e.g., substance use disorder).

In some embodiments, the experiment or assay used to determine increase translation of neurotrophic factors includes ELISA, western blot, immunofluorescence assays, proteomic experiments, and mass spectrometry. In some embodiments, the experiment or assay used to determine increase transcription of neurotrophic factors includes gene expression assays, PCR, and microarrays. In some embodiments, the experiment or assay used to determine increase secretion of neurotrophic factors includes ELISA, western blot, immunofluorescence assays, proteomic experiments, and mass spectrometry.

In some embodiments, the present invention provides a method for increasing at least one of translation, transcription or secretion of neurotrophic factors, comprising contacting a neuronal cell with a compound disclosed herein, such as a compound of Formula I, I-1, I-1A, I-1B, II, IIa, IIb, IIc, IId, IIe, IIf, IIg, Table 1, or a pharmaceutically acceptable salt thereof.

### Methods for Making Compounds

Exemplary compounds disclosed herein, including compounds of Formulas I, I-1, I-1A, I-1B, are prepared from the building blocks which are either commercially obtained, readily available, or otherwise prepared according to the compound and general schemes illustrated below:

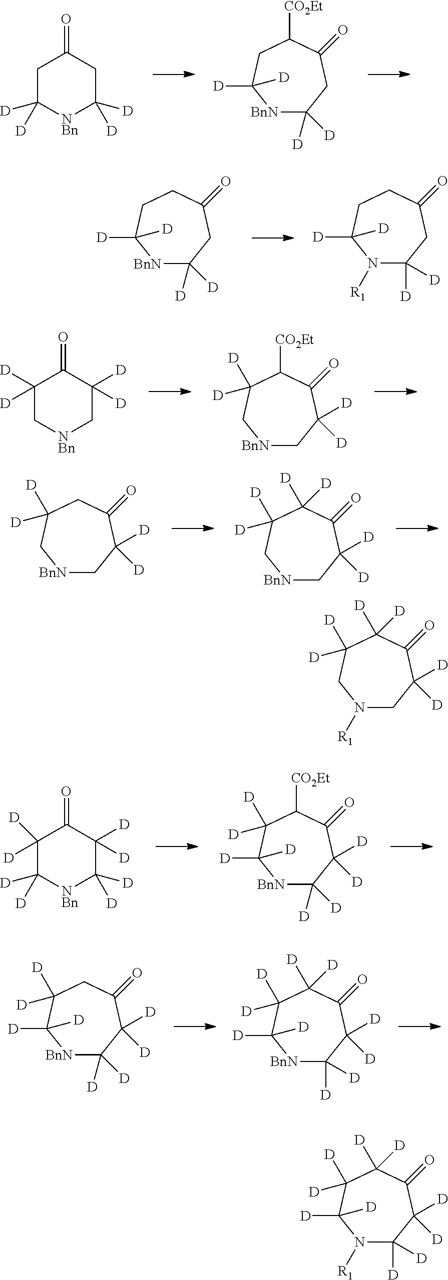

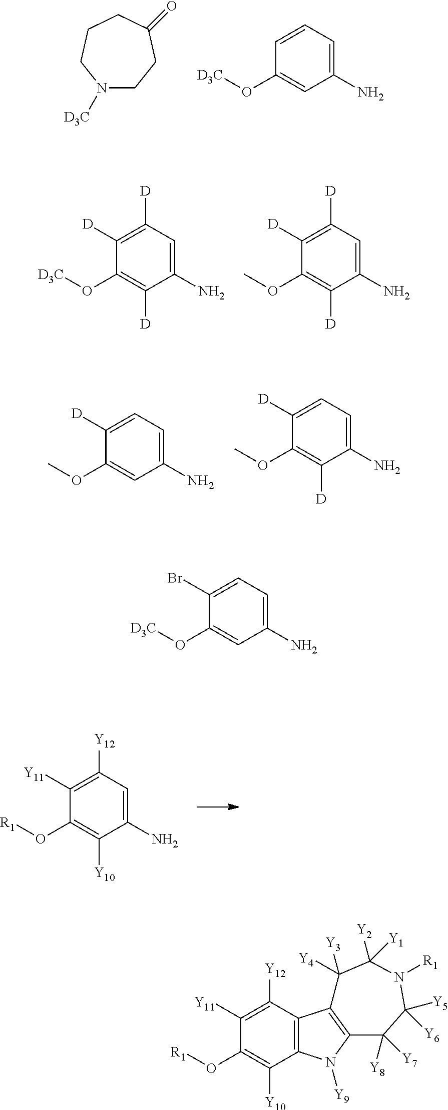

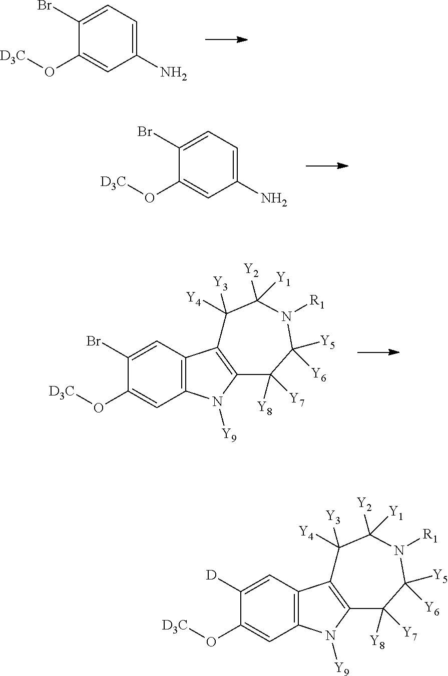

Exemplary compounds disclosed herein, including compounds of Formulas II, IIa, IIb, IIc, IId, IIe, IIf and IIg, Table 1, are prepared from the building blocks and according to the general schemes illustrate below:

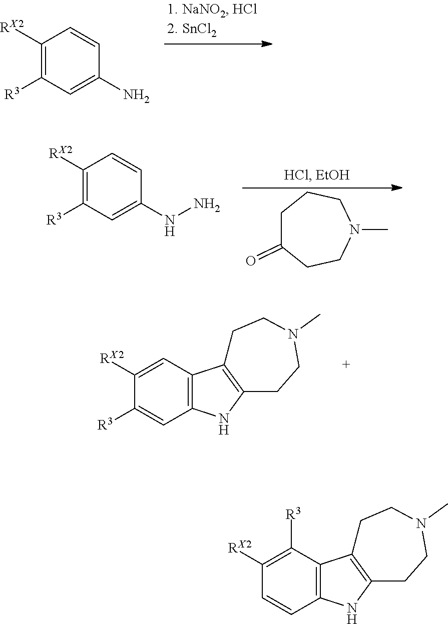

For example,

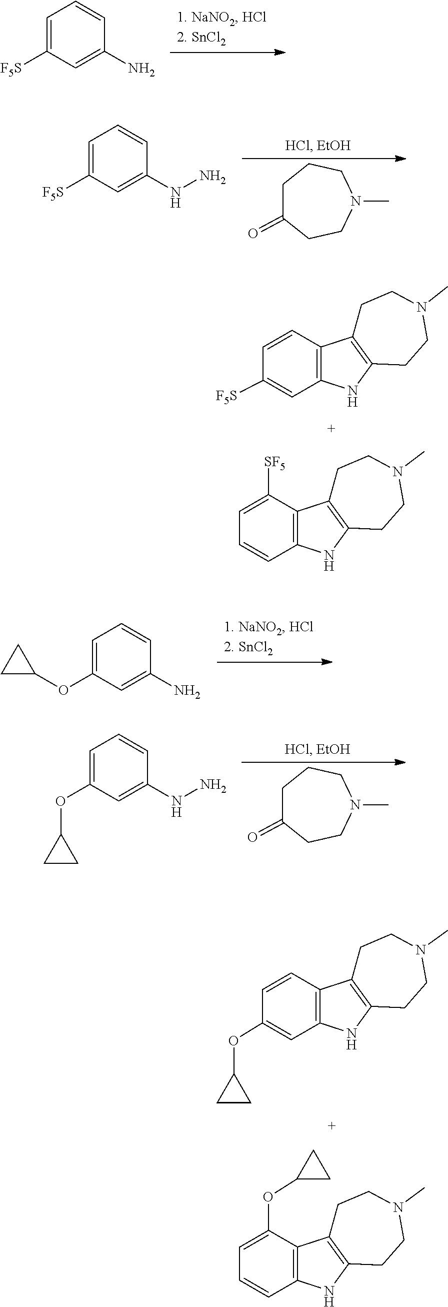

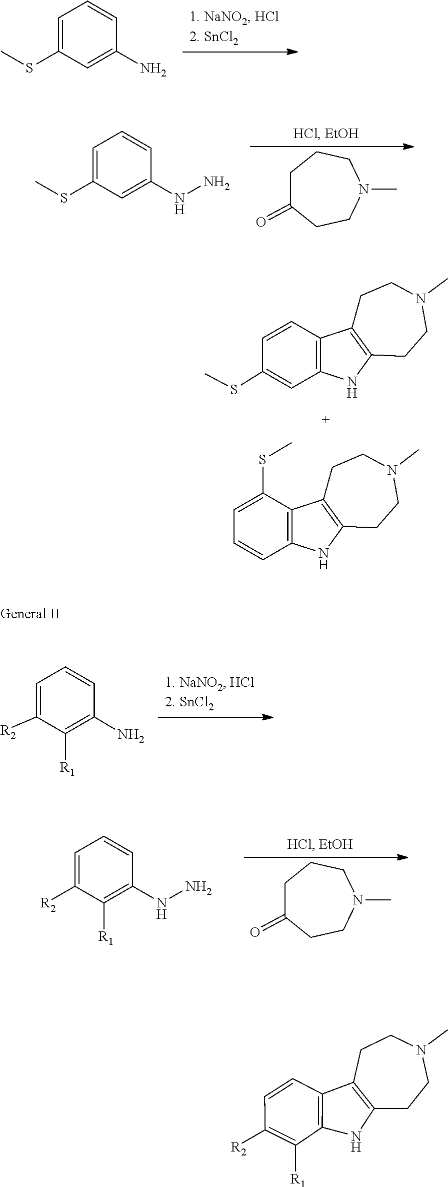

For example,

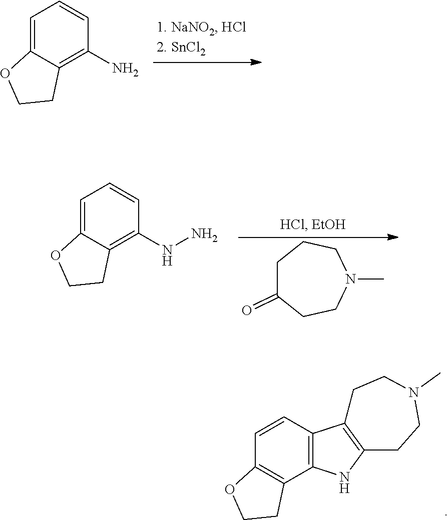

### Biological Assays

Microsomal Assay: Human liver microsomes (20 mg/mL) are obtained from Xenotech, LLC (Lenexa, KS). β-nicotinamide adenine dinucleotide phosphate, reduced form (NADPH), magnesium chloride (MgCl₂), and dimethyl sulfoxide (DMSO) are purchased from Sigma-Aldrich.

Determination of Metabolic Stability: 7.5 mM stock solutions of test compounds of the above structural formula (e.g., of an embodiment or aspect of embodiment thereof described herein), or pharmaceutically acceptable salt thereof, are prepared in DMSO. The 7.5 mM stock solutions are diluted to 12.5-5 μM in acetonitrile (ACN). The 20 mg/mL human liver microsomes are diluted to 0.625 mg/mL in 0.1 M potassium phosphate buffer, pH 7.4, containing 3 mM MgCl₂. The diluted microsomes are added to wells of a 96-well deep-well polypropylene plate in triplicate. A 10 pL aliquot of the 12.5-50 μM test compound is added to the microsomes and the mixture is pre-warmed for 10 minutes. Reactions are initiated by addition of pre-warmed NADPH solution. The final reaction volume is 0.5 mL and contains 4.0 mg/mL human liver microsomes, 0.25 μM test compound, and 2 mM NADPH in 0.1 M potassium phosphate buffer, pH 7.4, and 3 mM MgCl₂. The reaction mixtures are incubated at 37° C., and 50 pL aliquots are removed at 0, 5, 10, 20, and 30 minutes and added to shallow-well 96-well plates which contain 50 pL of ice-cold ACN (acetonitrile) with internal standard to stop the reactions. The plates are stored at 4° C. for 20 minutes after which 100 pL of water is added to the wells of the plate before centrifugation to pellet precipitated proteins. Supernatants are transferred to another 96-well plate and analyzed for amounts of parent remaining by LC-MS/MS using an Applied Bio-systems API 4000 mass spectrometer. The same procedure is followed for the non-deuterated counterpart of the compound and the positive control, 7-ethoxycoumarin (1 μM). Testing is done in triplicate.

Head-Twitch Response (HTR). The head-twitch response assay is performed as is known to those of skill in the art using both male and female C57BL/6J mice (3 per treatment). The mice are obtained and are approximately 8 weeks old at the time of the experiments. Compounds are administered via intraperitoneal injection (5 mL/kg) using 0.9% saline as the vehicle. As a positive control, Psilocybin was used. Behavior is videotaped, later scored by two blinded observers, and the results are averaged (Pearson correlation coefficient=0.93).

### EXAMPLES

### NMR Analysis Methodology

NMR analyses were conducted on a 400 MHz NMR spectrometer using deuterated chloroform, deuterated methanol or deuterated dimethyl sulfoxide as solvent. The shift (d) of each signal was measured in parts per million (ppm) relative to the residual solvent peak, and the multiplicity reported together with the associated coupling constant (J), where applicable.

### Agilent LC-MS Analysis Methodology

Instrument: Agilent 1260 infinity HPLC with Agilent 6130 single quadrupole mass spec.
Column: Phenomenex Kinetex XB-C18, 50×4.6 mm, 2.6 μm
Elution profile: See table below

| | % AQUEOUS (A) | |
|---|---|---|
| | (0.1% FORMIC ACID | % ORGANIC (B) |
| TIME (MINUTES) | IN WATER) | (100% ACETONITRILE) |
| 0 | 95 | 5 |
| 1.37 | 2 | 98 |
| 1.60 | 2 | 98 |
| 1.83 | 95 | 5 |
| 2.25 | 95 | 5 |

F low rate: 2 mL/min
Detector wavelength: 225±50 nm bandwidth
Column temperature: 40° C.
Injection volume: 1 μl
Mass spec parameters: Scanning in ES+/−& APCI over 70-1000 m/z
Needle wash: MeOH wash in vial 4, autosampler set up to do 5 needle washes (to wash the outside of the needle prior to injecting the sample).
Sample preparation: 0.5-1.0 mg/ml in either acetonitrile or DMSO depending on the nature of the sample in terms of solubility.

### Waters Alliance LCMS Method Details

Acidic method
Instrument: Waters 2795 Alliance HPLC system equipped with a 2996 PDA detector and
Micromass ZQ mass spectrometer detector.
Column: Gemini C18, 5 μm, 110 Å, 50×4.6 mm ID
Mobile phase A: 0.1% Formic acid in water
Mobile phase B: 100% acetonitrile
Gradient program (overall run time per injection is 8 minutes):

| TIME | MOBILE PHASE A | MOBILE PHASE B |
|---|---|---|
| 0.0 | 95 | 5 |
| 0.6 | 95 | 5 |
| 6.50 | 2 | 98 |
| 7.50 | 2 | 98 |
| 7.60 | 95 | 5 |
| 8.00 | 95 | 5 |

Flow rate: 1 ml/min
Injection volume: 10 μl
Column oven temperature: 40° C.
Detector: PDA UV at 190-400 nm, also fixed λ at 225 nm
Mass spec parameters: MS scan in ES+, ES-, ranging from M/Z 100-1000
Purge solvent involved in injection

In one embodiment, the compounds of the present disclosure are prepared as provided in the Examples.

### Example 1—Synthesis of 3-methyl-8-(trideuteriomethoxy)-2,4,5,6-tetrahydro-1H-azepino[4,5-b]indole (compound 1)

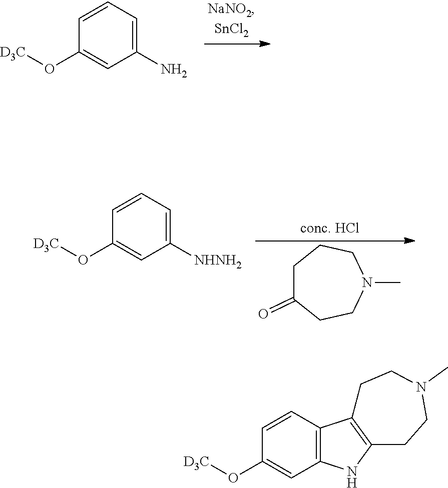

### Step 1: Preparation of [3-(trideuteriomethoxy)phenyl]hydrazine

To a stirred mixture of 3-(trideuteriomethoxy)aniline HCl salt (0.50 g, 3.1 mmol) in 6M HCl (4 mL) in an ice bath (0-5° C.) was added dropwise a solution of NaNO₂ (0.233 g, 3.38 mmol) in H₂O (2 mL) over 5 min. The mixture was stirred for 45 mins with ice bath cooling and then a solution of SnCl₂ (1.75 g, 9.22 mmol) in 6M HCl (4 mL) was added dropwise over 5 min. The mixture was stirred with ice bath cooling for 3 h, then filtered to remove the small amount of a brown precipitate. The filtrate was diluted with H₂O (50 mL) and basified to pH ˜14 with careful addition of 2M NaOH and extracted with EtOAc. The extracts were dried over Na₂SO₄, filtered and the filtrate was concentrated in vacuo to afford the title compound (0.41 g, 94%) as a viscous oil. The material was used in the next step without further purification. RT=0.435 min; m/z=[M+H]⁺ calculated for C₇H₇D₃N₂O 142.0. found 142.2; ¹H NMR (400 MHz, CDCl₃) δ 7.21-7.06 (m, 1H), 6.46-6.31 (m, 3H), 5.17 (br. s, 1H), 3.56 (br. s, 2H).

### Step 2: Preparation of 3-Methyl-8-(trideuteriomethoxy)-2,4,5,6-tetrahydro-1H-azepino[4,5-b]indole

To a mixture of [3-(trideuteriomethoxy)phenyl]hydrazine (200 mg, 1.42 mmol) in IMS (4 mL) was added 1-methylazepan-4-one HCl (232 mg, 1.42 mmol), followed by 12M HCl (0.71 mL, 8.5 mmol). The mixture was heated to 80° C. under microwave irradiation (Biotage Initiator+microwave) and stirred for 2 h. The mixture was poured into H₂O (100 mL), basified to pH ˜14 by addition of 2M NaOH and extracted with EtOAc (2×50 mL). The combined organic layers were dried over Na₂SO₄, filtered and the filtrate was concentrated in vacuo. The crude residue was purified by preparative-HPLC (eluent: 10% to 100% MeCN in H₂O). Pure fractions by LCMS were combined and lyophilised to afford the title compound (40 mg, 12%) as a solid. Retention time 1.056 min; Purity by UV (225 nm+/−50)=99.0%; m/z=[M+H]⁺ calculated for C₁₄H₁₅D₃N₂O 234.1. found 234.2; ¹H NMR (400 MHz, CDCl₃) δ 7.57 (br. s, 1H), 7.32 (d, J=8.5 Hz, 1H), 6.83-6.77 (m, 1H), 6.75 (dd, J=8.5, 2.3 Hz, 1H), 2.95-2.87 (m, 4H), 2.87-2.80 (m, 4H), 2.51 (s, 3H).

### Example 2—Synthesis of 8-(trideuteriomethoxy)-1,2,3,4,5,6-hexahydroazepino[4,5-b]indole (compound 163)

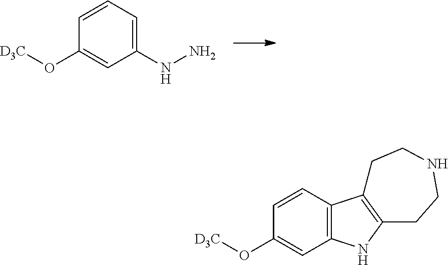 [SMILES: [2H]C([2H])([2H])Oc1cccc(NN)c1 ; [2H]C([2H])([2H])Oc1ccc2c3c([nH]c2c1)CCNCC3]

To a stirred mixture of azepan-4-one HCl (513 mg, 3.43 mmol) and [3-(trideuteriomethoxy)phenyl]hydrazine (440 mg, 3.12 mmol) in AcOH (10 mL) at rt was added ZnCl₂ (1.06 g, 7.79 mmol) portion wise over 2 min. the mixture was heated to 100° C. and stirred for 2 h, then cooled and concentrated in vacuo (to ca. 3 mL) and diluted with cold H₂O (100 mL). the solution was basified to pH ˜14 with 2M NaOH and extracted with EtOAc (3×50 mL). The combined organic layers were dried over Na₂SO₄, filtered and the filtrate was concentrated in vacuo to afford the crude product (as a ca. 2:1 mixture of regioisomers; 93% LCMS purity) (477 mg, 69%) as a dark grey solid. A portion of this crude material (70 mg) was purified by preparative HPLC (eluent: 19-29% MeCN in 0.1% aq. formic acid gradient). Pure fractions by LCMS were combined and lyophilised to afford the title compound (24 mg) as a solid. Retention time 1.07 min; m/z=[M+H]⁺ calculated for C₁₃H₁₃D₃N₂₀ 220.1. found 220.2; ¹H NMR (400 MHz, CD₃OD) δ 8.53 (s, 1H), 7.29 (dd, J=8.7, 0.6 Hz, 1H), 6.82 (dd, J=2.3, 0.6 Hz, 1H), 6.68 (dd, J=8.6, 2.3 Hz, 1H), 3.49-3.39 (m, 4H), 3.24-3.16 (m, 2H), 3.16-3.09 (m, 2H).

### Example 3—Synthesis of 8-methoxy-3-(trideuteriomethyl)-2,4,5,6-tetrahydro-1H-azepino[4,5-b]indole (compound 2)

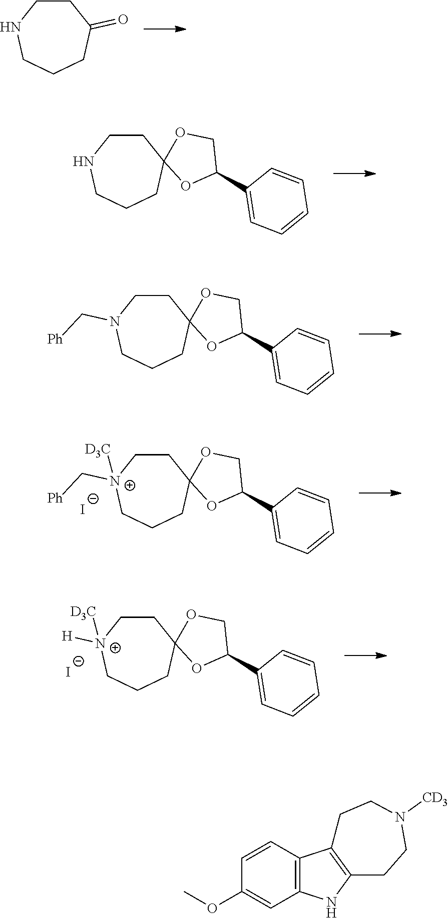

### Step 1: Preparation of (3R)-3-Phenyl-1,4-dioxa-9-azaspiro[4.6]undecane

A stirred mixture of azepan-4-one hydrochloride (4 g, 26.7 mmol) and (1R)-1-phenylethane-1,2-diol (3.80 g, 27.5 mmol) with pTsOH (0.1 g, 0.5 mmol) in toluene (60 ml) was heated to reflux with a Dean-Stark trap and stirred for 23 h. The mixture was cooled to rt, saturated sodium bicarbonate solution (100 mL) added, and the mixture extracted with DCM (2×100 mL). The combined organic layers were washed with brine (150 mL), dried (MgSO₄), filtered and the filtrate was concentrated in vacuo. The crude residue was purified by column chromatography on silica gel (eluent: 0 to 15% MeOH in DCM gradient) to afford the title compound (1.21 g, 19%) as an oil, and as a mixture of diastereoisomers. Retention time 1.629 min and 1.839 min (10 minute LCMS method); m/z=[M+H]⁺ calculated for C₁₄H₁₉NO₂ 234.1. Found 234.2 and 234.2; ¹H NMR (400 MHz, CDCl₃) δ 9.52 (br. s, 1H), 7.36-7.17 (m, 5H), 5.07-5.00 (m, 1H), 4.31-4.26 (m, 1H), 3.71-3.62 (m, 1H), 3.36-3.30 (m, 4H), 2.35-2.28 (m, 2H), 2.13-2.00 (M, 4H).

### Step 2: Preparation of (3R)-9-benzyl-3-phenyl-1,4-dioxa-9-azaspiro[4.6]undecane

To a stirred mixture of (3R)-3-phenyl-1,4-dioxa-9-azaspiro[4.6]undecane (1.2 g, 5.14 mmol) and benzaldehyde (0.525 mL, 5.14 mmol) in DCM (25 mL) at rt was added NaBH(OAc)₃ (1.63 g, 7.72 mmol) in one portion, and the mixture was stirred at rt overnight. The reaction mixture was quenched by careful addition of concentrated sodium bicarbonate solution (10 mL), followed by DCM (50 mL). The aqueous layer was extracted with DCM (40 mL) and the combined organic layers were concentrated in vacuo and the crude residue was purified by column chromatography on silica gel (eluent: 5% MeOH with 1% ammonium hydroxide in DCM) to afford the title compound (970 mg, 58%) as an oil. Retention time 1.310 min; m/z=[M+H]⁺ calculated for C₂₁H₂₅NO₂ 324.1. Found 324.2; ¹H NMR (400 MHz, CDCl₃) δ 7.41-7.18 (m, 10H), 5.07-4.91 (m, 1H), 4.25 (ddd, J=8.2, 6.1, 3.4 Hz, 1H), 3.71-3.55 (m, 3H), 2.81 (dt, J=11.4, 5.2 Hz, 1H), 2.74-2.51 (m, 3H), 2.20-1.94 (m, 4H), 1.78 (dqd, J=7.3, 4.7, 2.6 Hz, 2H).

### Step 3: Preparation of (3R)-9-Benzyl-3-phenyl-9-(trideuteriomethyl)-1,4-dioxa-9-azoniaspiro[4.6]undecane iodide

To a stirred mixture of (3R)-9-benzyl-3-phenyl-1,4-dioxa-9-azaspiro[4.6]undecane (965 mg, 2.98 mmol) in acetone (20 mL) at rt was added trideuterio(iodo)methane (0.241 mL, 3.88 mmol) in one portion. The mixture was stirred at rt overnight, then concentrated in vacuo to afford the title compound (1.39 g, 99%) as yellow foamy solid, and as a mixture of diastereomers. Retention time 0.329 min and 1.306 min; m/z=[M+H]⁺ calculated for C₂₂H₂₄D₃NO₂ 341.2. Found 341.2; ¹H NMR (400 MHz, DMSO-d₆) δ 7.65-7.26 (m, 10H), 5.19-5.07 (m, 1H), 4.60 (d, J=2.5 Hz, 2H), 4.43-4.29 (m, 1H), 3.73-3.21 (m, 4H), 2.44-2.19 (m, 2H), 2.06-1.76 (m, 4H).

### Step 4: Preparation of (3R)-3-Phenyl-9-(trideuteriomethyl)-1,4-dioxa-9-azoniaspiro[4.6]undecane iodide

To a mixture of 9-benzyl-3-phenyl-9-(trideuteriomethyl)-1,4-dioxa-9-azoniaspiro[4.6]undecane iodide (300 mg, 0.64 mmol) in MeOH (10 mL) was added 10% Pd/C (68.2 mg, 0.064 mmol). The suspension was stirred under hydrogen (500 psi) at rt overnight, then the temperature was increased to 45° C. and the mixture was stirred for 72 h. The mixture was filtered through a pad of celite and the filter cake was washed with MeOH (2×10 mL). The filtrate was concentrated in vacuo to afford the title compound (0.25 g, 100%) as an oil, that was used in the next step without further purification. Retention time 0.327 min and 1.130 min; m/z=[M+H]⁺ calculated for C₁₅H₁₈D₃NO₂ 251.1. Found 251.2; ¹H NMR (400 MHz, DMSO-d₆) δ 7.44-7.26 (m, 5H), 5.07 (ddd, J=8.7, 6.1, 2.7 Hz, 1H), 4.32 (ddd, J=8.9, 6.1, 2.9 Hz, 1H), 3.60 (t, J=8.3 Hz, 1H), 3.41-3.06 (m, 5H), 2.37-1.94 (m, 4H), 1.82 (s, 2H).

### Step 5: Preparation of 8-methoxy-3-(trideuteriomethyl)-2,4,5,6-tetrahydro-1H-azepino[4,5-b]indole

To a mixture of 9-(trideuteriomethyl)-1,4-dioxa-9-azoniaspiro[4.6]undecane iodide (240 mg, 0.794 mmol) in IMS (1.25 mL) was added (3-methoxyphenyl)hydrazine hydrochloride (139 mg, 0.794 mmol), followed by 12M HCl (0.397 mL, 4.77 mmol). The mixture was heated to 80° C. under microwave irradiation and stirred for 2.5 h, then H₂O (50 mL) added, basified to pH ˜14 by addition of 2M NaOH solution, and extracted with EtOAc (2×50 mL). The combined organic layers were dried over Na₂SO₄, filtered and the filtrate was concentrated in vacuo. The crude residue was purified by preparative HPLC (eluent: 10-20% MeCN in 0.1% aq formic acid gradient). Pure fractions were combined and lyophilised to afford the title compound (10 mg, 5%) as an oil. Retention time 1.061 min; m/z=[M+H]⁺ calculated for C₁₄H₁₅D₃N₂₀ 234.1. found 234.2; ¹H NMR (400 MHz, CDCl₃) δ 7.85 (s, 1H), 7.47 (d, J=8.6 Hz, 1H), 6.91 (dd, J=2.2, 1.1 Hz, 1H), 6.84 (d, J=2.3 Hz, 1H), 6.79 (dd, J=8.6, 2.3 Hz, 1H), 3.84 (s, 3H), 3.02-2.84 (m, 2H), 2.70-2.56 (m, 2H), 2.34 (s, 6H).

### Example 4—Synthesis of 8-(trideuteriomethoxy)-3-(trideuteriomethyl)-2,4,5,6-tetrahydro-1H-azepino[4,5-b]indole (compound 3)

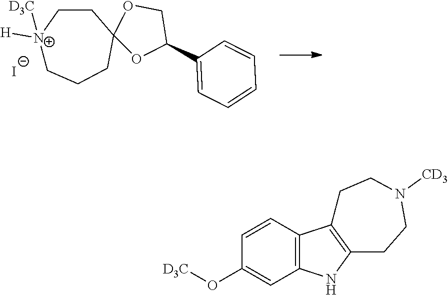 [SMILES: [2H]C([2H])([2H])[NH+]1CCCC2(CC1)OC[C@@H](c1ccccc1)O2 ; [I-] ; [2H]C([2H])([2H])Oc1ccc2c3c([nH]c2c1)CCN(C([2H])([2H])[2H])CC3]

To a mixture of [3-(trideuteriomethoxy)phenyl]hydrazine (112 mg, 0.794 mmol) in IMS (1.25 mL) was added 9-(trideuteriomethyl)-1,4-dioxa-9-azoniaspiro[4.6]undecane iodide (240 mg, 0.794 mmol), followed by 12M HCl (0.397 ml, 4.77 mmol). The mixture was heated to 80° C. under microwave irradiation and stirred for 2.5 h, then H₂O (50 mL) added, basified to pH ˜14 by addition of 2M NaOH solution, and extracted with EtOAc (2×50 mL). The combined organic layers were dried over Na₂SO₄, filtered and the filtrate was concentrated in vacuo. The crude residue was purified by preparative HPLC (eluent: 10-20% MeCN in 0.1% aq formic acid gradient). Pure fractions were combined and lyophilised to afford the title compound (5.5 mg, 2%) as an oil. Retention time 1.072 min; m/z=[M+H]⁺ calculated for C₁₄H₁₂D₆N₂₀ 237.1. found 237.1; ¹H NMR (400 MHz, CD₃OD) δ 8.55 (s, 1H), 7.28 (d, J=8.6 Hz, 1H), 6.82 (d, J=2.2 Hz, 1H), 6.68 (dd, J=8.7, 2.3 Hz, 1H), 3.46 (td, J=7.7, 5.1 Hz, 4H), 3.24-3.07 (m, 4H).

### Example 5—Synthesis of 8-(cyclopropoxy)-3-methyl-2,4,5,6-tetrahydro-1H-azepino[4,5-b]indole (compound 117)

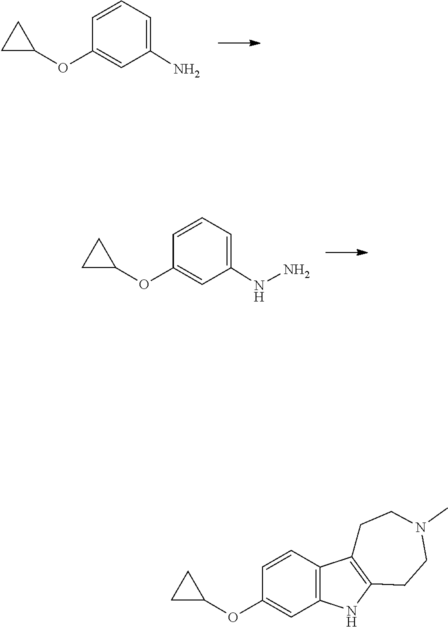 [SMILES: Nc1cccc(OC2CC2)c1 ; NNc1cccc(OC2CC2)c1 ; CN1CCc2[nH]c3cc(OC4CC4)ccc3c2CC1]

### Step 1: Preparation of [3-(cyclopropoxy)phenyl]hydrazine

To a stirred mixture of 3-(cyclopropoxy)aniline [CAS No: 1202359-26-9](0.225 g, 1.51 mmol) in 6M HCl (2 mL) in an ice-water bath was added a solution of NaNO₂ (0.114 g, 1.66 mmol) in H₂O (1 mL) dropwise over 5 min. The reaction mixture was stirred for 45 min with ice-water bath cooling, then a solution of SnCl₂ (0.858 g, 4.52 mmol) in 6M HCl (2 mL) was added dropwise over 5 min. The mixture was warmed to rt and stirred for 3 h, then filtered to remove a small amount of solid. The filtrate was diluted with H₂O (50 mL) and basified to pH ˜14 with careful addition of 2M NaOH. The mixture was extracted with DCM (2×50 mL) and the combined organic extracts were dried over Na₂SO₄, filtered and the filtrate was concentrated in vacuo to afford the title compound (213 mg, 86%) as a viscous oil. The crude product was used in the next step without further purification. Retention time 1.00 min; m/z=[M+H]⁺ calculated for C₉H₁₂N₂₀ 165.0. found 165.2; ¹H NMR (400 MHz, CDCl₃) δ 7.13 (td, J=7.9, 0.7 Hz, 1H), 6.58-6.45 (m, 2H), 6.45-6.34 (m, 1H), 5.16 (s, 1H), 3.76-3.67 (m, 1H), 3.55 (s, 2H), 0.78-0.73 (m, 4H).

### Step 2: Preparation of 8-(cyclopropoxy)-3-methyl-2,4,5,6-tetrahydro-1H-azepino[4,5-b]indole

To a mixture of [3-(cyclopropoxy)phenyl]hydrazine (210 mg, 1.28 mmol) in industrial methylated spirits (IMS; 1.5 mL) was added 1-methylazepan-4-one HCl (209 mg, 1.28 mmol), followed by 12M HCl (0.64 mL). The mixture was heated to 80° C. under microwave irradiation (Biotage Initiator+microwave) and stirred for 2.5 h. After cooling, the mixture was poured into ice-H₂O (50 mL), basified to pH ˜14 by addition of 2M NaOH and extracted with DCM (2×50 mL). The combined organic layers were concentrated in vacuo and the crude residue was purified by preparative HPLC (eluent: 0% to 100% MeCN in H₂O). Pure fractions by LCMS were combined and lyophilised to afford the title compound (23 mg, 7%) as a solid. Retention time 1.19 min; m/z=[M+H]⁺ calculated for C₁₆H₂₀N₂₀ 257.1. found 257.2; ¹H NMR (400 MHz, CDCl₃) δ 7.58 (s, 1H), 7.31 (d, J=8.6 Hz, 1H), 7.00 (d, J=2.2 Hz, 1H), 6.82 (dd, J=8.6, 2.2 Hz, 1H), 3.75 (tt, J=6.0, 3.3 Hz, 1H), 2.96-2.86 (m, 4H), 2.86-2.77 (m, 4H), 2.51 (s, 3H), 0.82-0.70 (m, 4H).

### Example 6—Synthesis of 8-cyclopropoxy-3-(²H₃)methyl-1H,2H,3H,4H,5H,6H-azepino[4,5-b]indole (compound 145)

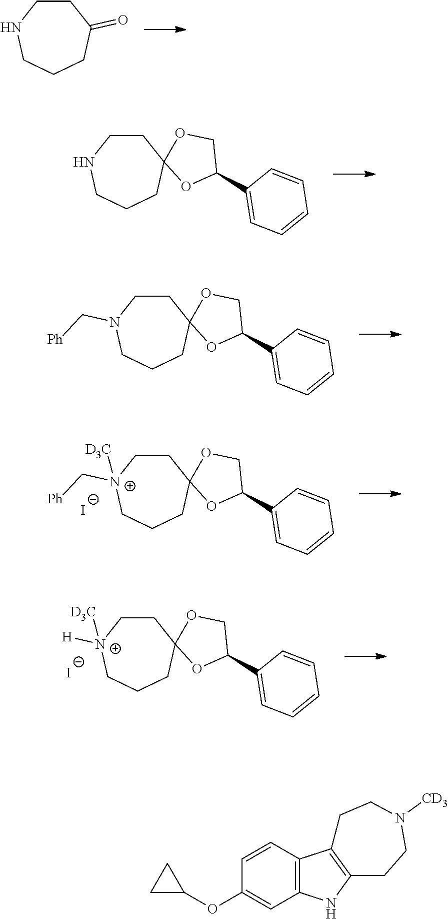

### Step 1: Preparation of (3R)-3-Phenyl-1,4-dioxa-9-azaspiro[4.6]undecane

A stirred mixture of azepan-4-one hydrochloride (4 g, 26.7 mmol) and (1R)-1-phenylethane-1,2-diol (3.80 g, 27.5 mmol) with pTsOH (0.1 g, 0.5 mmol) in toluene (60 ml) was heated to reflux with a Dean-Stark trap and stirred for 23 h. The mixture was cooled to rt, saturated sodium bicarbonate solution (100 mL) added, and the mixture extracted with DCM (2×100 mL). The combined organic layers were washed with brine (150 mL), dried (MgSO₄), filtered and the filtrate was concentrated in vacuo. The crude residue was purified by column chromatography on silica gel (eluent: 0 to 15% MeOH in DCM gradient) to afford the title compound (1.21 g, 19%) as an oil, and as a mixture of diastereoisomers. Retention time 1.629 min and 1.839 min (10 minute LCMS method); m/z=[M+H]⁺ calculated for C₁₄H₁₉NO₂ 234.1. Found 234.2 and 234.2; ¹H NMR (400 MHz, CDCl₃) δ 9.52 (br. s, 1H), 7.36-7.17 (m, 5H), 5.07-5.00 (m, 1H), 4.31-4.26 (m, 1H), 3.71-3.62 (m, 1H), 3.36-3.30 (m, 4H), 2.35-2.28 (m, 2H), 2.13-2.00 (M, 4H).

### Step 2: Preparation of (3R)-9-benzyl-3-phenyl-1,4-dioxa-9-azaspiro[4.6]undecane

To a stirred mixture of (3R)-3-phenyl-1,4-dioxa-9-azaspiro[4.6]undecane (1.2 g, 5.14 mmol) and benzaldehyde (0.525 mL, 5.14 mmol) in DCM (25 mL) at rt was added NaBH(OAc)₃ (1.63 g, 7.72 mmol) in one portion, and the mixture was stirred at rt overnight. The reaction mixture was quenched by careful addition of concentrated sodium bicarbonate solution (10 mL), followed by DCM (50 mL). The aqueous layer was extracted with DCM (40 mL) and the combined organic layers were concentrated in vacuo and the crude residue was purified by column chromatography on silica gel (eluent: 5% MeOH with 1% ammonium hydroxide in DCM) to afford the title compound (970 mg, 58%) as an oil. Retention time 1.310 min; m/z=[M+H]⁺ calculated for C₂₁H₂₅NO₂ 324.1. Found 324.2; ¹H NMR (400 MHz, CDCl₃) δ 7.41-7.18 (m, 10H), 5.07-4.91 (m, 1H), 4.25 (ddd, J=8.2, 6.1, 3.4 Hz, 1H), 3.71-3.55 (m, 3H), 2.81 (dt, J=11.4, 5.2 Hz, 1H), 2.74-2.51 (m, 3H), 2.20-1.94 (m, 4H), 1.78 (dqd, J=7.3, 4.7, 2.6 Hz, 2H).

### Step 3: Preparation of (3R)-9-Benzyl-3-phenyl-9-(trideuteriomethyl)-1,4-dioxa-9-azoniaspiro[4.6]undecane iodide

To a stirred mixture of (3R)-9-benzyl-3-phenyl-1,4-dioxa-9-azaspiro[4.6]undecane (965 mg, 2.98 mmol) in acetone (20 mL) at rt was added trideuterio(iodo)methane (0.241 mL, 3.88 mmol) in one portion. The mixture was stirred at rt overnight, then concentrated in vacuo to afford the title compound (1.39 g, 99%) as yellow foamy solid, and as a mixture of diastereomers. Retention time 0.329 min and 1.306 min; m/z=[M+H]⁺ calculated for C₂₂H₂₄D₃NO₂ 341.2. Found 341.2; ¹H NMR (400 MHz, DMSO-d₆) δ 7.65-7.26 (m, 10H), 5.19-5.07 (m, 1H), 4.60 (d, J=2.5 Hz, 2H), 4.43-4.29 (m, 1H), 3.73-3.21 (m, 4H), 2.44-2.19 (m, 2H), 2.06-1.76 (m, 4H).

### Step 4: Preparation of (3R)-3-Phenyl-9-(trideuteriomethyl)-1,4-dioxa-9-azoniaspiro[4.6]undecane iodide

To a mixture of 9-benzyl-3-phenyl-9-(trideuteriomethyl)-1,4-dioxa-9-azoniaspiro[4.6]undecane iodide (300 mg, 0.64 mmol) in MeOH (10 mL) was added 10% Pd/C (68.2 mg, 0.064 mmol). The suspension was stirred under hydrogen (500 psi) at rt overnight, then the temperature was increased to 45° C. and the mixture was stirred for 72 h. The mixture was filtered through a pad of celite and the filter cake was washed with MeOH (2×10 mL). The filtrate was concentrated in vacuo to afford the title compound (0.25 g, 100%) as an oil, that was used in the next step without further purification. Retention time 0.327 min and 1.130 min; m/z=[M+H]⁺ calculated for C₁₅H₁₈D₃NO₂ 251.1. Found 251.2; ¹H NMR (400 MHz, DMSO-d₆) δ 7.44-7.26 (m, 5H), 5.07 (ddd, J=8.7, 6.1, 2.7 Hz, 1H), 4.32 (ddd, J=8.9, 6.1, 2.9 Hz, 1H), 3.60 (t, J=8.3 Hz, 1H), 3.41-3.06 (m, 5H), 2.37-1.94 (m, 4H), 1.82 (s, 2H).

### Step 5: Preparation of 8-cyclopropoxy-3-(²H₃)methyl-1H,2H,3H,4H,5H,6H-azepino[4,5-b]indole

To a solution of [3-(cyclopropoxy)phenyl]hydrazine (105 mg, 0.64 mmol) in IMS (1.25 mL) was added (3R)-3-Phenyl-9-(trideuteriomethyl)-1,4-dioxa-9-azoniaspiro[4.6]undecane iodide (241 mg, 0.64 mmol), followed by concentrated HCl (12.0 mol/L, 0.319 mL, 3.82 mmol). The mixture was heated to 80° C. and stirred for 2.5 h under microwave irradiation, then diluted with H₂O (50 mL), basified to pH ˜14 by addition of 2M NaOH and extracted with EtOAc (2×50 mL). The combined organic layers were dried over Na₂SO₄, filtered and the filtrate was concentrated in vacuo. The crude residue was purified by reverse-phase preparative HPLC (eluent: 10-20% MeCN in 0.1% aq formic acid gradient). The purest fractions were combined and lyophilised to afford the title compound (7 mg, 3%) as a viscous oil. Retention time 1.203 min; m/z=[M+H]⁺ calculated for C₁₆H₁₇D₃N₂₀ 260.4. found 260.2; ¹H NMR (400 MHz, CD₃OD) δ 7.33-7.08 (m, 2H), 6.92 (d, J=2.2 Hz, 1H), 6.62 (dd, J=8.6, 2.2 Hz, 1H), 3.66 (tt, J=6.2, 3.0 Hz, 1H), 3.34-3.22 (m, 4H), 3.02 (dt, J=30.0, 5.4 Hz, 4H), 0.73-0.53 (m, 4H).

### Example 7—Synthesis of 3-cyclopropyl-8-methoxy-2,4,5,6-tetrahydro-1H-azepino[4,5-b]indole (compound 164)

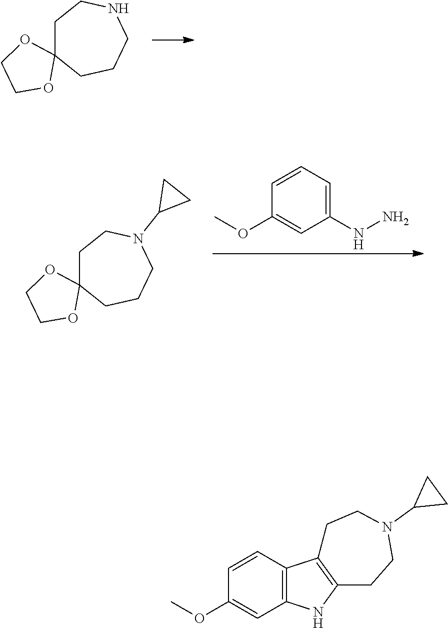

### Step 1: Preparation of 9-cyclopropyl-1,4-dioxa-9-azaspiro[4.6]undecane

A mixture of 1,4-dioxa-9-azaspiro[4.6]undecane HCl [CAS No: 947534-11-4](6.00 g, 31.0 mmol), AcOH (17.7 mL, 310 mmol), 3 Å molecular sieves (12 g) and (1-ethoxycyclopropoxy)-trimethyl-silane (21.6 g, 124 mmol) in MeOH (135 mL) was stirred for 5 min. NaCNBH₃ (5.84 g, 92.9 mmol) was added and the mixture was heated to 50° C. and stirred for 23 h. The mixture was cooled, diluted with saturated sodium bicarbonate solution (500 mL) and extracted with EtOAc (2×400 mL). The combined organic layers were washed with brine (400 mL), dried over MgSO₄, filtered and the filtrate was concentrated in vacuo. The crude residue was purified by column chromatography on silica gel (MeOH/DCM 1:49) to give the title compound (3.55 g, 58%) as an oil. Retention time 7.68 min; ¹H NMR (400 MHz, CDCl₃) δ 3.89 (s, 4H), 2.85-2.74 (m, 4H), 1.95-1.78 (m, 5H), 1.71-1.61 (m, 2H), 0.49-0.35 (m, 4H).

### Step 2: Preparation of 3-cyclopropyl-8-methoxy-2,4,5,6-tetrahydro-1H-azepino[4,5-b]indole

To a mixture of (3-methoxyphenyl)hydrazine HCl [CAS No: 39232-91-2](221 mg, 1.27 mmol) in IMS (4 mL) was added 9-cyclopropyl-1,4-dioxa-9-azaspiro[4.6]undecane (250 mg, 1.27 mmol), followed by 12M HCl (0.63 mL). The mixture was heated to 80° C. under microwave irradiation (Biotage Initiator+microwave) and stirred for 2.5 h. After cooling, H₂O (50 mL) was added, and the mixture was basified to pH ˜14 by addition of 2M NaOH and extracted with EtOAc (2×50 mL). The combined organic layers were dried over Na₂SO₄, filtered and the filtrate was concentrated in vacuo. The crude residue was purified by preparative HPLC (eluent: 19-29% MeCN in 0.1% aq. formic acid gradient). Pure fractions by LCMS were combined and lyophilised to afford the title compound (20 mg, 6%) as a solid. Retention time 1.15 min; m/z=[M+H]⁺ calculated for C₁₆H₂₀N₂₀ 257.1. found 257.2; ¹H NMR (400 MHz, CD₃OD) δ 8.34 (s, 1H), 7.27 (dd, J=8.6, 0.5 Hz, 1H), 6.81 (d, J=2.2 Hz, 1H), 6.67 (dd, J=8.6, 2.3 Hz, 1H), 3.79 (s, 3H), 3.49 (q, J=5.2 Hz, 4H), 3.19-3.12 (m, 2H), 3.11-3.04 (m, 2H), 2.79 (p, J=5.5 Hz, 1H), 0.94-0.88 (m, 4H).

### Example 8—Synthesis of 8-cyclopropoxy-3-cyclopropyl-1,2,3,4,5,6-hexahydroazepino[4,5-b]indole (compound 165)

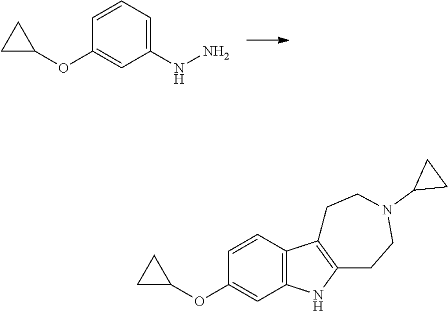 [SMILES: NNc1cccc(OC2CC2)c1 ; c1cc2c3c([nH]c2cc1OC1CC1)CCN(C1CC1)CC3]

To a mixture of [3-(cyclopropoxy)phenyl]hydrazine (116 mg, 0.71 mmol) in IMS (1 mL) was added 9-cyclopropyl-1,4-dioxa-9-azaspiro[4.6]undecane (139 mg, 0.71 mmol), followed by 12M HCl (0.35 mL). The mixture was heated to 80° C. under microwave irradiation (Biotage Initiator+microwave) and stirred for 2 h. After cooling, the mixture was poured into ice-H₂O (30 mL), the mixture was basified to pH ˜14 by addition of 2M NaOH and extracted with EtOAc (2×30 mL). The combined organic layers were concentrated in vacuo and the crude residue was purified by preparative HPLC (eluent: 19-29% MeCN in 0.1% aq. formic acid gradient). Product fractions were combined and lyophilized to afford the title compound (10 mg, 5%) as an oil. Retention time 1.23 min; m/z=[M+H]⁺ calculated for C₁₈H₂₂N₂O 283.1. found 283.2; ¹H NMR (400 MHz, CD₃OD) δ 8.41 (s, 1H), 7.32-7.22 (m, 1H), 7.05-6.98 (m, 1H), 6.77-6.67 (m, 1H), 3.82-3.71 (m, 1H), 3.38 (dt, J=5.7, 4.5 Hz, 4H), 3.14-3.08 (m, 2H), 3.07-2.99 (m, 2H), 2.64 (p, J=5.4 Hz, 1H), 0.83 (d, J=5.5 Hz, 4H), 0.80-0.72 (m, 2H), 0.72-0.64 (m, 2H).

### Example 9—Synthesis of 3-cyclopropyl-8-(trideuteriomethoxy)-2,4,5,6-tetrahydro-1H-azepino[4,5-b]indole (compound 146)

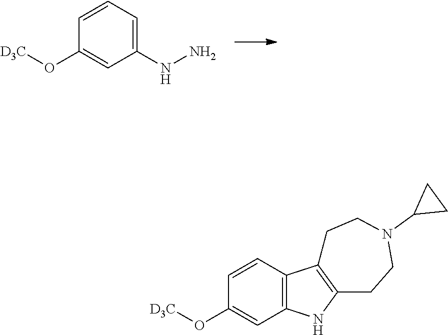 [SMILES: [2H]C([2H])([2H])Oc1cccc(NN)c1 ; [2H]C([2H])([2H])Oc1ccc2c3c([nH]c2c1)CCN(C1CC1)CC3]

To a mixture [3-(trideuteriomethoxy)phenyl]hydrazine (197 mg, 1.39 mmol) in IMS (4 mL) was added 9-cyclopropyl-1,4-dioxa-9-azaspiro[4.6]undecane (250 mg, 1.27 mmol), followed by 12M HCl (0.63 mL). The mixture was heated to 80° C. under microwave irradiation (Biotage Initiator+microwave) and stirred for 2.5 h. After cooling, H₂O (50 mL) was added, and the mixture was basified to pH ˜14 by addition of 2M NaOH and extracted with EtOAc (2×50 mL). The combined organic layers were dried over Na₂SO₄, filtered and the filtrate was concentrated in vacuo. The crude residue was purified by preparative HPLC (eluent: 19-29% MeCN in 0.1% aq. formic acid gradient). Pure fractions by LCMS were combined and lyophilised to afford the title compound (50 mg, 15%) as a solid. Retention time 1.14 min; m/z=[M+H]⁺ calculated for C₁₆H₁₇D₃N₂₀ 260.1. found 260.2; ¹H NMR (400 MHz, CD₃OD) δ 7.27 (dd, J=8.6, 0.6 Hz, 1H), 6.80 (dd, J=2.3, 0.5 Hz, 1H), 6.66 (dd, J=8.6, 2.3 Hz, 1H), 3.47-3.39 (m, 4H), 3.13 (dd, J=6.5, 4.6 Hz, 2H), 3.09-3.01 (m, 2H), 2.71 (p, J=5.5 Hz, 1H), 0.87 (d, J=5.5 Hz, 4H).

### Example 10—Synthesis of pentafluoro-(3-methyl-2,4,5,6-tetrahydro-1H-azepino[4,5-b]indol-8-yl)-λ⁶-sulfane (compound 109) and pentafluoro-(3-methyl-2,4,5,6-tetrahydro-1H-azepino[4,5-b]indol-10-yl)-λ⁶-sulfane (compound 110)

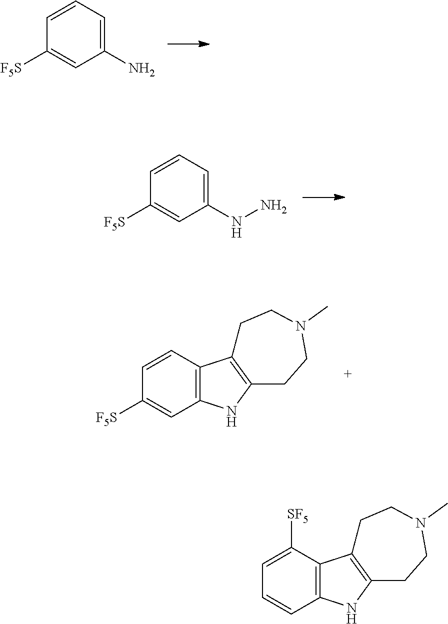

Step 1: Preparation of [3-(Pentafluoro-λ⁶-sulfanyl)phenyl]hydrazine A mixture of 3-(pentafluoro-λ⁶-sulfanyl)aniline (0.50 g, 2.28 mmol) was suspended in 6M HCl (5 mL) and stirred for 15 min. The suspension was cooled to at −5 to 0° C. in an ice/MeOH bath and a solution of NaNO₂ (0.173 g, 2.51 mmol) in H₂O (2 mL) was added dropwise. The resulting suspension was stirred for 15 min at 0° C., giving a clear solution. A mixture of SnCl₂ (1.30 g, 6.84 mmol) in 6M HCl (3 mL) was then added dropwise, giving an immediate precipitate. The mixture was stirred at 0° C. for 3 h, then filtered to remove a small amount of solid. The filtrate was diluted with H₂O (50 mL) and basified to pH ˜14 with careful addition of 2M NaOH and the mixture was extracted with EtOAc (3×50 mL). The combined organic layers were dried over Na₂SO₄, filtered and the filtrate was concentrated in vacuo to afford the title compound (0.40 g, 74%) as a viscous oil. The crude product was used in the next step without further purification. ¹H NMR (400 MHz, d₆-DMSO) δ 7.30-7.18 (m, 3H), 7.00-6.93 (m, 2H), 4.15 (s, 2H).

### Step 2: Preparation of pentafluoro-(3-methyl-2,4,5,6-tetrahydro-1H-azepino[4,5-b]indol-8-yl)-λ⁶-sulfane and pentafluoro-(3-methyl-2,4,5,6-tetrahydro-1H-azepino[4,5-b]indol-10-yl)-λ⁶-sulfane

To a mixture of 1-methylazepan-4-one HCl (307 mg, 1.88 mmol) and [3-(pentafluoro-λ⁶-sulfanyl)phenyl]hydrazine (0.40 mg, 1.71 mmol) in AcOH (10 mL) at rt was added ZnCl₂ (582 mg, 4.27 mmol) portion wise over 2 min. The mixture was heated to 100° C. and stirred for 2 h, then concentrated in vacuo (to ca. 3 mL) and diluted with cold H₂O (100 mL). The mixture was basified to pH ˜14 with 2M NaOH, giving a fine precipitate that was removed by filtration. The filtrate was extracted with EtOAc (3×50 mL) and the combined organic layers were dried over Na₂SO₄, filtered, and the filtrate was concentrated in vacuo. The crude product was purified by preparative HPLC (eluent: 25-35% MeCN in 0.1% aq formic acid gradient). Pure fractions by LCMS were combined and lyophilised to afford the title compound (32 mg, 6%) as a solid. Retention time 1.30 min; m/z=[M+H]⁺ calculated for C₁₃H₁₅F₅N₂S 327.6. found 327.0; ¹H NMR (400 MHz, DMSO-d₆) δ 11.28 (s, 1H), 8.20 (s, 1H), 7.73 (d, J=2.1 Hz, 1H), 7.62-7.49 (m, 1H), 7.40 (dd, J=8.8, 2.1 Hz, 1H), 3.01-2.94 (m, 2H), 2.85 (dd, J=6.4, 3.7 Hz, 2H), 2.77 (ddd, J=10.2, 6.2, 3.9 Hz, 4H), 2.44 (s, 3H). Also isolated was pentafluoro-(3-methyl-2,4,5,6-tetrahydro-1H-azepino[4,5-b]indol-10-yl)-λ⁶-sulfane (24 mg, 4%) as a solid. Retention time 1.29 min; m/z=[M+H]⁺ calculated for C₁₃H₁₅F₅N₂S 327.6; 327.0; ¹H NMR (400 MHz, DMSO-d₆) δ 11.66 (s, 1H), 8.19 (s, 1H), 7.62-7.52 (m, 2H), 7.12 (t, J=8.0 Hz, 1H), 3.08-3.02 (m, 2H), 3.02-2.95 (m, 2H), 2.71-2.60 (m, 4H), 2.36 (s, 3H).

### Example 11—Synthesis of 8-methyl-2,3,5,6,7,8,9,10-octahydroazepino[4,5-b]furo[2,3-f]indole (compound 166)

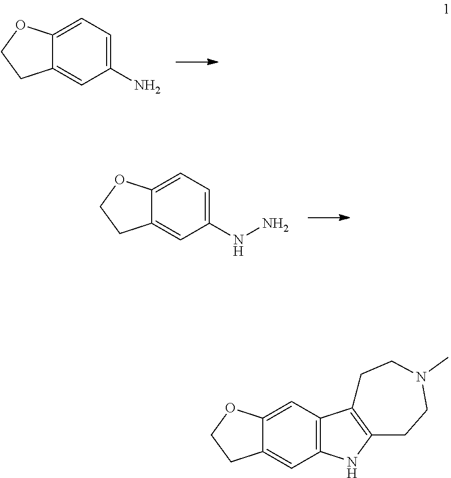 [SMILES: Nc1ccc2c(c1)CCO2 ; NNc1ccc2c(c1)CCO2 ; CN1CCc2[nH]c3cc4c(cc3c2CC1)OCC4]

### Step 1: Preparation of 2,3-dihydrobenzofuran-5-yl-hydrazine

To a mixture of 2,3-dihydrobenzofuran-5-amine [CAS No: 42933-43-7](0.205 g, 1.52 mmol) in 6M HCl (2 mL) in an ice-water bath was added a mixture of NaNO₂ (0.115 g, 1.67 mmol) in H₂O (1 mL) dropwise over 5 min. The mixture was stirred for 45 min with ice-H₂O bath cooling and then a mixture of SnCl₂ (0.863 g, 4.55 mmol) in 6M HCl (2 mL) was added dropwise over 5 min. The mixture was stirred with ice-H₂O bath cooling for 3 h, then diluted with H₂O (50 mL) and basified to pH ˜14 with careful addition of 2M NaOH. The mixture was extracted with EtOAc (2×50 mL) and the combined organic layers were dried over Na₂SO₄, filtered and the filtrate was concentrated in vacuo to afford the title compound (174 mg, 76%) as a viscous oil. The crude product was used immediately in the next step without further purification.

### Step 2: Preparation of 8-methyl-2,3,5,6,7,8,9,10-octahydroazepino[4,5-b]furo[2,3-f]indole

To a mixture of 2,3-dihydrobenzofuran-5-yl-hydrazine_(171 mg, 1.14 mmol) in IMS (4 mL) was added 1-methylazepan-4-one HCl (186 mg, 1.14 mmol), followed by 12M HCl (0.57 mL). The mixture was heated to 80° C. under microwave irradiation (Biotage Initiator+microwave) and stirred for 2.5 h. After cooling, H₂O (50 mL) was added, and the mixture was basified to pH ˜14 by addition of 2M NaOH and extracted with DCM (2×50 mL). The combined organic layers were concentrated in vacuo and the crude product was purified by preparative HPLC (eluent: 0% to 100% MeCN in H₂O). Pure fractions by LCMS were combined and lyophilised to afford the title compound (6 mg, 2%) as a solid. Retention time 1.07 min; m/z=[M+H]⁺ calculated for C₁₅H₁₈N₂O 243.1; 243.2; ¹H NMR (400 MHz, CD₃OD) δ 7.05 (d, J=1.1 Hz, 1H), 6.66 (d, J=0.7 Hz, 1H), 4.47 (t, J=8.3 Hz, 2H), 3.21 (ddd, J=9.2, 8.3, 1.1 Hz, 2H), 2.98-2.90 (m, 2H), 2.88-2.77 (m, 6H), 2.49 (s, 3H).

### Example 12—Synthesis of 8-methyl-1,2,6,7,8,9,10,11-octahydroazepino[4,5-b]furo[2,3-g]indole (compound 124)

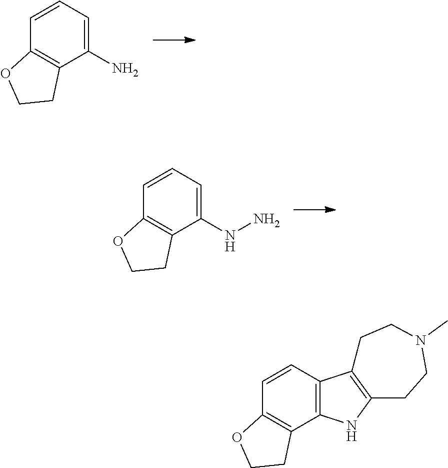 [SMILES: Nc1cccc2c1CCO2 ; NNc1cccc2c1CCO2 ; CN1CCc2[nH]c3c4c(ccc3c2CC1)OCC4]

### Step 1: Preparation of (2,3-dihydrobenzofuran-4-yl)hydrazine

To a mixture of 2,3-dihydrobenzofuran-4-amine [CAS No: 61090-37-7](0.20 g, 1.48 mmol) in 6M HCl (2 mL) in an ice-H₂O bath was added NaNO₂ (0.112 g, 1.63 mmol) in H₂O (1 mL) dropwise over 5 min. The mixture was stirred for 45 mins with ice-H₂O bath cooling, then a mixture of SnCl₂ (0.842 g, 4.44 mmol) in 6M HCl (2 mL) was added dropwise over 5 min. The mixture was warmed to rt and stirred for 3 h, then diluted with H₂O (50 mL) and basified to pH ˜14 with careful addition of 2M NaOH. The mixture was extracted with EtOAc (2×50 mL) and the combined organic layers were dried over Na₂SO₄, filtered and the filtrate was concentrated in vacuo to afford the title compound (190 mg, 85%) as an oil. The crude product was used in the next step without further purification. ¹H NMR (400 MHz, CDCl₃) δ 7.13-6.99 (m, 1H), 6.48 (d, J=8.0 Hz, 1H), 6.35 (d, J=8.0 Hz, 1H), 4.96 (s, 1H), 4.62-4.55 (m, 2H), 3.60 (s, 2H), 2.99 (t, J=8.7 Hz, 2H).

### Step 2: Preparation of 8-methyl-1,2,6,7,8,9,10,11-octahydroazepino[4,5-b]furo[2,3-g]indole

To a mixture of (2,3-dihydrobenzofuran-4-yl)hydrazine (190 mg, 1.27 mmol) in IMS (4 mL) was added 1-methylazepan-4-one HCl (207 mg, 1.27 mmol), followed by 12M HCl (0.63 mL). The mixture was heated to 80° C. under microwave irradiation (Biotage Initiator+microwave) and stirred for 2 h. The mixture was concentrated in vacuo (to ca. 2 mL) and added to H₂O (50 mL), basified to pH ˜14 by addition of 2M NaOH, and extracted with EtOAc (2×50 mL). The combined organic layers were dried over Na₂SO₄, filtered and the filtrate was concentrated in vacuo. The crude residue was purified by preparative HPLC (eluent: 10% to 100% MeCN in water) and pure fractions by LCMS were combined and lyophilised to afford the title compound (45 mg, 12%) as a solid. Retention time 1.07 min; m/z=[M+H]⁺ calculated for C₁₅H₁₈N₂O 243.1; 243.2; ¹H NMR (400 MHz, CDCl₃) δ 7.38 (s, 1H), 7.19 (d, J=8.4, 1H), 6.69 (d, J=8.4 Hz, 1H), 4.64 (t, J=8.6 Hz, 2H), 3.28 (t, J=8.6 Hz, 2H), 2.97-2.80 (m, 8H), 2.51 (s, 3H).

### Example 13—Synthesis of 9-methyl-2,3,7,8,9,10,11,12-octahydro-1H-azepino[4,5-b]pyrano[2,3-g]indole (compound 122)

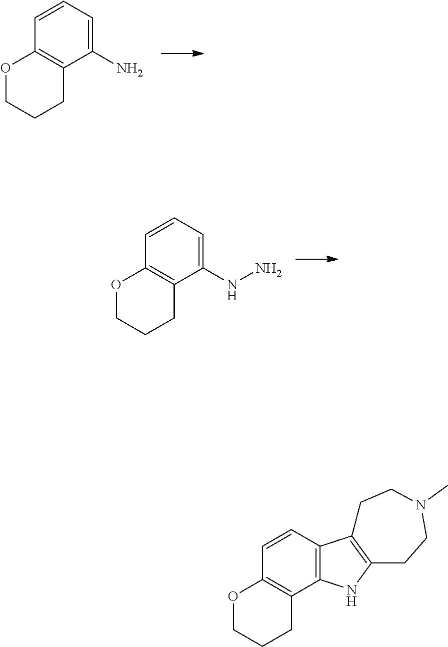 [SMILES: Nc1cccc2c1CCCO2 ; NNc1cccc2c1CCCO2 ; CN1CCc2[nH]c3c4c(ccc3c2CC1)OCCC4]

### Step 1: Preparation of chroman-5-ylhydrazine

To a mixture of chroman-5-amine HCl [CAS No: 1965309-15-2](0.200 g, 1.08 mmol) in 6M HCl (2 mL) in an ice-H₂O bath was added a mixture of NaNO₂ (82 mg, 1.19 mmol) in H₂O (1 mL) dropwise over 5 min. The mixture was stirred for 45 min with ice-H₂O bath cooling and then a mixture of SnCl₂ (0.613 g, 3.23 mmol) in 6M HCl (2 mL) was added dropwise over 5 min. The mixture was warmed to rt and stirred for 3 h, then diluted with H₂O (50 mL) and basified to pH ˜14 with careful addition of 2M NaOH. The mixture was extracted with EtOAc (3×50 mL) and the combined organic layers were dried over Na₂SO₄, filtered and the filtrate was concentrated in vacuo to afford the title compound (165 mg, 93%) as a viscous oil. The crude product was used in the next step without further purification. ¹H NMR (400 MHz, CDCl₃) δ 7.13-6.98 (m, 1H), 6.61-6.44 (m, 1H), 6.41-6.33 (m, 1H), 4.22-4.04 (m, 2H), 2.56-2.45 (m, 2H), 2.09-2.04 (m, 2H).

### Step 2: Preparation of 9-methyl-2,3,7,8,9,10,11,12-octahydro-1H-azepino[4,5-b]pyrano[2,3-g]indole

To a mixture of chroman-5-ylhydrazine (165 mg, 1.00 mmol) in IMS (3 mL) was added 1-methylazepan-4-one HCl (164 mg, 1.00 mmol), followed by 12M HCl (0.50 mL). The mixture was heated to 80° C. under microwave irradiation (Biotage Initiator+microwave) and stirred for 2 h. After cooling, H₂O (50 mL) was added, and the mixture was basified to pH ˜14 by addition of 2M NaOH and extracted with EtOAc (2×50 mL). The combined organic layers were dried over Na₂SO₄, filtered and the filtrate was concentrated in vacuo. The crude residue was purified by preparative HPLC (eluent: 10% to 100% MeCN in H₂O) and pure fractions by LCMS were combined and lyophilised to afford the title compound (36 mg, 14%) as a solid. Retention time 1.14 min; m/z=[M+H]⁺ calculated for C₁₆H₂₀N₂₀ 257.1; 257.2; ¹H NMR (400 MHz, CDCl₃) δ 7.43 (s, 1H), 7.18 (dd, J=8.6, 0.8 Hz, 1H), 6.64 (d, J=8.5 Hz, 1H), 4.24-4.16 (m, 2H), 2.98-2.91 (m, 2H), 2.91-2.76 (m, 8H), 2.51 (s, 3H), 2.13-2.05 (m, 2H).

### Example 14—Synthesis of 3-methyl-2,4,5,6-tetrahydro-1H-azepino[4,5-b]indole-8-carbonitrile (compound 136) and 3-methyl-2,4,5,6-tetrahydro-1H-azepino[4,5-b]indole-10-carbonitrile (compound 138)

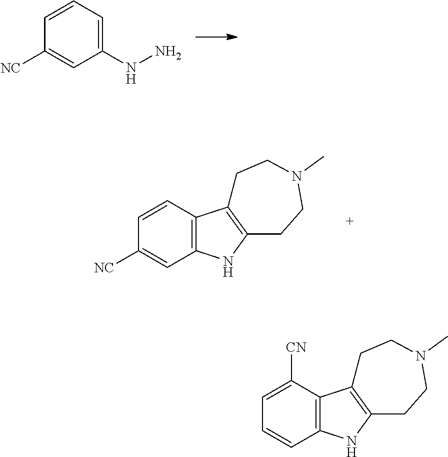 [SMILES: N#Cc1cccc(NN)c1 ; CN1CCc2[nH]c3cc(C#N)ccc3c2CC1 ; CN1CCc2[nH]c3cccc(C#N)c3c2CC1]

To a stirred mixture of 3-hydrazinobenzonitrile (428 mg, 3.21 mmol) in TFA (15 mL) at rt was added 1-methylazepan-4-one hydrochloride (526 mg, 3.21 mmol) portion wise over 2 min. The mixture was heated to 110° C. and stirred for 2 h under microwave irradiation, then concentrated to ca. 5 mL in vacuo and poured onto ice-H₂O (100 mL). The solution was basified to pH ˜14 by careful portion wise addition of NaOH and extracted with EtOAc (3×50 mL). The combined organic layers were concentrated in vacuo and the crude residue was purified by preparative HPLC (eluent: 19-29% MeCN in 0.1% aq formic acid gradient). Pure fractions by LCMS were combined and lyophilised to afford 3-methyl-2,4,5,6-tetrahydro-1H-azepino[4,5-b]indole-8-carbonitrile (8 mg, 1%) as a solid. Retention time 1.063 min; m/z=[M+H]⁺ calculated for C₁₄H₁₅N₃ 226.1. found 226.2; ¹H NMR (400 MHz, CDCl₃) δ 8.37 (s, 1H), 7.61 (d, J=1.1 Hz, 1H), 7.47 (d, J=8.3 Hz, 1H), 7.40-7.27 (m, 1H), 3.28 (d, J=6.1 Hz, 1H), 3.24-3.03 (m, 7H), 2.70 (s, 3H). Also isolated from the column was 3-methyl-2,4,5,6-tetrahydro-1H-azepino[4,5-b]indole-10-carbonitrile (6 mg, 0.8%) as a viscous oil. Retention time 1.097 min; m/z=[M+H]⁺ 20 calculated for C₁₄H₁₅N₃ 226.1. found 226.2; ¹H NMR (400 MHz, CD₃OD) δ 7.60 (dd, J=8.2, 0.9 Hz, 1H), 7.42 (dd, J=7.4, 1.0 Hz, 1H), 7.18 (dd, J=8.2, 7.4 Hz, 1H), 3.52 (dd, J=7.0, 3.8 Hz, 2H), 3.49-3.42 (m, 4H), 3.28 (dd, J=6.6, 4.4 Hz, 2H), 2.96 (s, 3H).

### Example 15—Synthesis of 2-(8-methoxy-2,4,5,6-tetrahydro-1H-azepino[4,5-b]indol-3-yl)ethanol (compound 140)

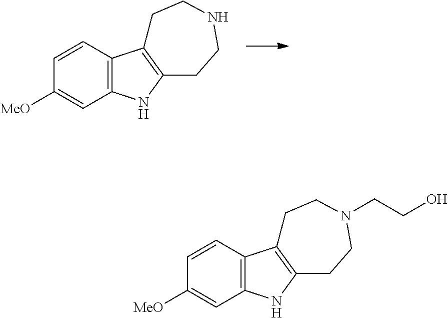 [SMILES: COc1ccc2c3c([nH]c2c1)CCNCC3 ; COc1ccc2c3c([nH]c2c1)CCN(CCO)CC3]

To a mixture of 8-methoxy-1,2,3,4,5,6-hexahydroazepino[4,5-b]indole [CAS No: 15918-87-3](as a ca. 2:1 mixture with the 10-methoxy regioisomer, 65.0 mg, 0.30 mmol) in MeCN (1 mL) was added K₂CO₃ (62.3 mg, 0.45 mmol) in one portion at rt, followed by 2-iodoethanol (62 mg, 0.36 mmol) in one portion. The mixture was heated to 40° C. and stirred for 4 h, then cooled, added to H₂O (25 mL), and basified to pH ˜14 with 2M NaOH. The mixture was extracted with EtOAc (2×25 mL) and the combined organic layers were dried over Na₂SO₄, filtered and the filtrate was concentrated in vacuo. The crude residue was purified by preparative HPLC (eluent: 19-29% MeCN in 0.1% aq formic acid gradient). Pure fractions by LCMS were combined and lyophilised to afford the title compound (24 mg, 30%) as a solid. Retention time 1.064 min; m/z=[M+H]⁺ calculated for C₁₅H₂₀N₂₀₂ 261.1. found 261.2; ¹H NMR (400 MHz, CD₃OD) δ 8.47 (s, 1H), 7.28 (dd, J=8.7, 0.5 Hz, 1H), 6.82 (d, J=2.2 Hz, 1H), 6.68 (dt, J=8.6, 2.1 Hz, 1H), 4.09 (s, 1H), 4.04-3.90 (m, 2H), 3.80 (s, 3H), 3.60 (dt, J=9.4, 5.6 Hz, 3H), 3.44-3.37 (m, 2H), 3.27-3.19 (m, 2H), 3.18-3.11 (m, 2H).

### Example 16—Synthesis of 8-methoxy-3-(2-methylsulfonylethyl)-2,4,5,6-tetrahydro-1H-azepino[4,5-b]indole (compound 143)

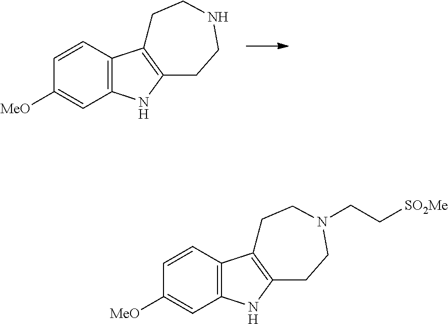 [SMILES: COc1ccc2c3c([nH]c2c1)CCNCC3 ; COc1ccc2c3c([nH]c2c1)CCN(CCS(C)(=O)=O)CC3]

To a stirred mixture of 8-methoxy-1,2,3,4,5,6-hexahydroazepino[4,5-b]indole (as a ca. 2:1 mixture with the 10-methoxy regioisomer, 140 mg, 0.65 mmol) in MeOH (3 mL) at rt was added 1-methylsulfonylethylene (0.068 mL, 0.78 mmol) dropwise over 1 min. The mixture was stirred at 25° C. for 4 h, then concentrated in vacuo and the crude residue was purified by preparative HPLC (eluent: 19-29% MeCN in 0.1% aq formic acid gradient). Pure fractions by LCMS were combined and lyophilised to afford the title compound (27 mg, 12%) as a solid. Retention time 1.104 min; m/z=[M+H]⁺ calculated for C₁₆H₂₂N₂O₃S 323.1. found 323.0; ¹H NMR (400 MHz, CD₃OD) δ 8.17 (s, 1H), 7.24 (dd, J=8.7, 0.5 Hz, 1H), 6.79 (d, J=2.2 Hz, 1H), 6.65 (dd, J=8.6, 2.3 Hz, 1H), 3.79 (s, 3H), 3.48 (dd, J=7.7, 6.2 Hz, 2H), 3.45-3.33 (m, 2H), 3.18 (q, J=5.0 Hz, 4H), 3.09 (d, J=0.7 Hz, 3H), 3.07-3.00 (m, 2H), 2.99-2.92 (m, 2H).

### Example 17—Synthesis of 2-(8-methoxy-2,4,5,6-tetrahydro-1H-azepino[4,5-b]indol-3-yl)-N,N-dimethyl-ethanamine (compound 144)

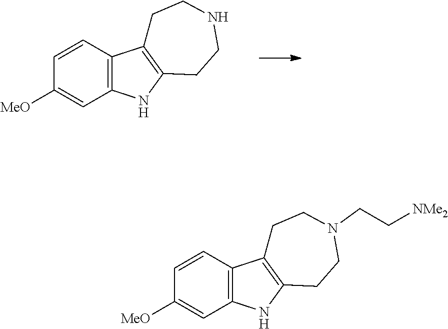 [SMILES: COc1ccc2c3c([nH]c2c1)CCNCC3 ; COc1ccc2c3c([nH]c2c1)CCN(CCN(C)C)CC3]

To a stirred mixture of 8-methoxy-1,2,3,4,5,6-hexahydroazepino[4,5-b]indole (as a ca. 2:1 mixture with the 10-methoxy regioisomer, 100 mg, 0.46 mmol) in MeCN (2 mL) was added NaI (69.3 mg, 0.46 mmol) and K₂CO₃ (192 mg, 1.39 mmol) in single portions at rt, followed by 2-chloro-N,N-dimethyl-ethanamine hydrochloride (79.9 mg, 0.56 mmol) portion wise over 1 min. The mixture was heated to 40° C. and stirred at rt overnight, then diluted with MeCN (1 mL) and the inorganic solids were removed by filtration. The filtrate was concentrated in vacuo and the crude residue was purified by preparative HPLC (eluent: 19-29% MeCN in 0.1% aq formic acid gradient). Pure fractions by LCMS were combined and lyophilised to afford the title compound (19 mg, 11%) as a solid. Retention time 0.693 min; m/z=[M+H]⁺ calculated for C₁₇H₂₅N₃O 288.1. found 288.2; ¹H NMR (400 MHz, DMSO-d₆) δ 10.46 (s, 1H), 7.23 (d, J=8.5 Hz, 1H), 6.75 (d, J=2.2 Hz, 1H), 6.60 (dd, J=8.6, 2.3 Hz, 1H), 3.73 (s, 3H), 3.72-2.80 (m, 12H, obscured by residual water peak), 2.76 (s, 6H).

### Example 18—General Method for Deuteration of Indole Ring: Synthesis of 7,9-Dideuterio-8-methoxy-3-methyl-2,4,5,6-tetrahydro-1H-azepino[4,5-b]indole (compound 167)

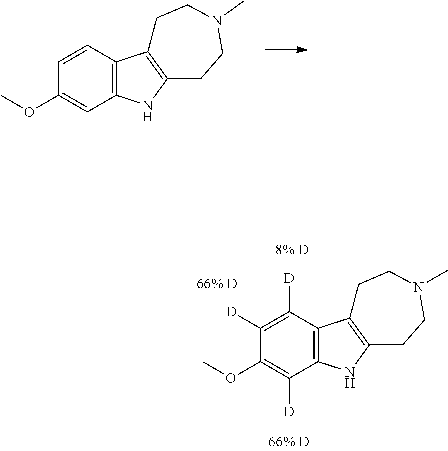

To stirred deuteriotrifluoromethanesulfonate (0.556 mL, 6.25 mmol) at 0° C. was added 8-methoxy-3-methyl-2,4,5,6-tetrahydro-1H-azepino[4,5-b]indole (40.0 mg, 0.174 mmol) in one portion under a blanket of nitrogen. The reaction vessel was sealed under nitrogen and the solution was stirred for 20 h at 20° C. The reaction mixture was then added dropwise to ice/water (50 mL) and the resulting solution was basified by portion-wise addition of sodium bicarbonate. The solution was extracted with EtOAc (2×50 mL) and the combined organic layers were dried over Na2SO4, filtered and the filtrate was concentrated in vacuo. The crude product was purified by column chromatography on silica gel to afford the product (28 mg, 69%) as a colourless viscous oil. Retention time 1.091 min; m/z=[M+H]⁺ calculated for C₁₄H₁₅D₂N₂O 232.2. found 233.2; ¹H NMR (400 MHz, CD₃OD) δ 7.35-7.22 (m, 1H), 6.83 (s, 0.35H), 6.69 (dd, J=8.7, 1.6 Hz, 0.35H), 3.79 (s, 3H), 3.49 (dt, J=10.4, 5.4 Hz, 4H), 3.20 (dd, J=6.5, 4.7 Hz, 2H), 3.12 (dd, J=6.5, 4.5 Hz, 2H), 3.01 (s, 3H). As can be seen from the Scheme above, the approximate degree of deuteration of the aromatic protons was 66% at the 7- and 9-positions, together with approximately 8% at the 10-position. It is envisaged that complete deuteration at the 7- and 9-positions could be obtained by resubjecting the product to the reaction conditions. Separately, other starting materials can be utilized in the reaction to introduce deuterium on the benzenoid ring of indole. Other starting materials include those that already include deuterium elsewhere in the molecule.

Additional methods and starting materials for making the present compounds will be readily apparent to those of skill in the art upon consideration of the present disclosure.

### Example 18—Evaluation of Metabolic Stability in Human Liver Microsomes

Microsomal Assay: Human liver microsomes (20 mg/mL) are obtained from Xenotech, LLC (Lenexa, KS). β-nicotinamide adenine dinucleotide phosphate, reduced form (NADPH), magnesium chloride (MgCl₂), and dimethyl sulfoxide (DMSO) are purchased from Sigma-Aldrich.

Determination of Metabolic Stability: 7.5 mM stock solutions of test compounds of the above structural formula (e.g., of an embodiment or aspect of embodiment thereof described herein), or pharmaceutically acceptable salt thereof, are prepared in DMSO. The 7.5 mM stock solutions are diluted to 12.5-5 μM in acetonitrile (ACN). The 20 mg/mL human liver microsomes are diluted to 0.625 mg/mL in 0.1 M potassium phosphate buffer, pH 7.4, containing 3 mM MgCl₂. The diluted microsomes are added to wells of a 96-well deep-well polypropylene plate in triplicate. A 10 pL aliquot of the 12.5-50 μM test compound is added to the microsomes and the mixture is pre-warmed for 10 minutes. Reactions are initiated by addition of pre-warmed NADPH solution. The final reaction volume is 0.5 mL and contains 4.0 mg/mL human liver microsomes, 0.2 μM test compound, and 2 mM NADPH in 0.1 M potassium phosphate buffer, pH 7.4, and 3 mM MgCl₂. The reaction mixtures are incubated at 37° C., and 50 pL aliquots are removed at 0, 5, 10, 20, and 30 minutes and added to shallow-well 96-well plates which contain 50 pL of ice-cold ACN (acetonitrile) with internal standard to stop the reactions. The plates are stored at 4° C. for 20 minutes after which 100 pL of water is added to the wells of the plate before centrifugation to pellet precipitated proteins. Supernatants are transferred to another 96-well plate and analyzed for amounts of parent remaining by LC-MS/MS using an Applied Bio-systems API 4000 mass spectrometer. The same procedure is followed for the non-deuterated counterpart of the compound and the positive control, 7-ethoxycoumarin (1 μM). Testing is done in triplicate.

Data analysis: The in vitro Ti/s for test compounds are calculated from the slopes of the linear regression of % parent remaining (In) vs incubation time relationship.

$$in vitro {T}_{1 / 2} = 0.693 / k$$

k=−[slope of linear regression of % parent remaining (In) vs incubation time]
The apparent intrinsic clearance is calculated using the following equation:

$${CL}_{int} ( mL / min / kg ) = ( 0.693 / in vitro T ) ( Incubation Volume / mg of microsomes ) ( 45 mg micorsomes / gram of liver ) ( 20 gm of liver / kg b . w . )$$

Data analysis is performed using Microsoft Excel Software.

In these experiments, values equal to or more than a 10% or 15% change in half-life are considered to be a significant difference if the apparent intrinsic clearance ratio (deuterated compound/Tabernanthalog (for Examples 1-4) and disclosed compound/comparator (for Examples 5-17)) is >1.15 or <0.85, then there is considered to be significant differentiation. Varied clearance rates and half-lives for psychedelic compounds are beneficial in order to expedite clearance of excess compound in order to prevent off target effects and reduce potential side effects. When paired with a therapy session, varied clearance rates and half-lives for psychedelic compounds are beneficial as they reduce the time commitment burden on the therapist.

TABLE 2
Metabolic stability in human liver microsomes
of representative deuterated compounds

| Compound | | CL_{int(mic)} | CLᵢₙₜ₍ₗᵢᵥₑᵣ₎ | T_{1/2} |
|---|---|---|---|---|
| Number | Compound ID | (μL/min/mg) | (mL/min/kg) | (min) |
| — | 8-methoxy-1,2,3,4,5,6- | 12.7 | 11.4 | 109.4 |
| | hexahydroazepino[4,5-b]indole | | | |
| 163 | 8-trideuteromethoxy-1,2,3,4,5,6- | 16.1 | 14.5 | 86.3 |
| | hexahydroazepino[4,5-b]indole | | | |
| 1 | 8-trideuteromethoxy-3-methyl- | 17.2 | 15.4 | 80.8 |
| | 1,2,3,4,5,6-hexahydroazepino[4,5- | | | |
| | b]indole | | | |
| 2 | 8-methoxy-3-trideuteromethyl- | 16.6 | 14.9 | 83.7 |
| | 1,2,3,4,5,6-hexahydroazepino[4,5- | | | |
| | b]indole | | | |
| 3 | 8-trideuteromethoxy-3- | 16.8 | 15.1 | 82.5 |
| | trideuteromethyl-1,2,3,4,5,6- | | | |
| | hexahydroazepino[4,5-b]indole | | | |
| — | 8-methoxy-3-methyl-1,2,3,4,5,6- | 15.6* | 14.0* | 90* |
| | hexahydroazepino[4,5-b]indole | | | |
| | (TBG) | | | |
| — | Testosterone | 114.4* | 102.9* | 12.2* |
| — | Diclofenac | 201.9* | 181.7* | 7.2* |
| — | Propafenone | 226.1* | 203.5* | 6.5* |

*average of two experiments
CL_{int(mic)}: intrinsic clearance
CL_{int(mic)} = 0.693/T_{1/2}/mg microsome protein per mL
CLᵢₙₜ₍ₗᵢᵥₑᵣ₎ = CL_{int(mic)} * mg microsomal protein/g liver weight * g liver weight/kg body weight
T_{1/2}: half life
Compound 163 was compared to 8-methoxy-1,2,3,4,5,6-hexahydroazepino[4,5-b]indole.
Compounds 1, 2 and 3 were compared to TBG.

Based on the results in Table 2, 8-trideuteromethoxy-1,2,3,4,5,6-hexahydroazepino[4,5-b]indole (Compound 163) demonstrates a significant difference in half-life and intrinsic clearance ratio of >1.15 compared with 8-methoxy-1,2,3,4,5,6-hexahydroazepino[4,5-b]indole. Additionally, Compounds 1, 2 and 3 demonstrate differences in half-life and intrinsic clearance compared to TBG.

### Example 19—Oral Bioavailability in Rats

Pharmacokinetics of test articles following a single intravenous or oral administration in rats: A pharmacokinetic (PK) study is performed in three male Sprague-Dawley (SD) rats following intravenous (IV) and oral (PO) administration of Tabernanthalog (TBG), test compounds, or test deuterated compounds—Tabernanthalog (TBG), at 1 mg/kg (IV) and 10 (PO) mg/kg. Test compounds, or Tabernanthalog (TBG), are measured in plasma. A detailed description of the in vivo methods:

### Regulatory

All animal experiments are performed under UK Home Office Licenses and with local ethical committee clearance. All experiments will be performed by technicians that have completed parts A and B of the Home Office Personal License course and hold a current personal license. All experiments are performed in dedicated Biohazard 2 facilities with full AAALAC accreditation.

### Rat Strain

Rats used in these studies are supplied by Charles River (Margate UK) and are specific pathogen free. The strain of rats is Sprague Dawley. Male rats are 175-225 g on receipt and are allowed to acclimatize for 5-7 days.

### Animal Housing

Rats are group housed in sterilised individual ventilated cages that expose the animals at all times to HEPA filtered sterile air. Animals will have free access to food and water (sterile) and will have sterile aspen chip bedding (at least once weekly). The room temperature is 22° C.+/−1° C., with a relative humidity of 60% and maximum background noise of 56 dB. Rats will be exposed to 12 hour light/dark cycles.

### Treatment

Test article is diluted 10% v/v DMSO, 40% v/v PEG-400, 50% v/v Water. The test articles are administered in a dose volume of 2 mL/kg for intravenous (IV) and 5 mL/kg (PO) for oral routes of administration.

### Example 20—Single IV/PO Dose Pharmacokinetics Study in Rats

Each test article is administered as a single IV bolus (via a lateral tail-vein) or a single oral gavage in cohorts of 3 rats per route. Following dose administrations, a 100 μL whole blood sample (EDTA) is collected via the tail-vein at time-points described in Table 3. The blood is centrifuged to separate plasma. Approximately 40 μL of plasma is dispensed per time-point, per rat, in a 96 well plate and frozen until analysis. Bioanalysis is carried out on plasma samples.

TABLE 3
Single IV and oral dose pharmacokinetics
profile of test articles in rat plasma

| | | | | Blood sample | |
|---|---|---|---|---|---|
| | | | Dose | collection | No. of |
| Group | Test article | Route | (mg/kg) | (post dose) | rats |
| 1 | Tabernanthalog | IV | 1 | 5 min, 15 min, | 3 |
| | (TBG) | | | 30 min, 1 h, 2 h, | |
| | | | | 4 h, 7 h, 24 h | |
| 2 | Tabernanthalog | PO | 10 | 15 min, 30 min, | 3 |
| | (TBG) | | | 45 min, 1 h, 2 h, | |
| | | | | 4 h, 7 h, 24 h | |
| 3 | Test Article or | IV | 1 | 5 min, 15 min, | 3 |
| | Deuterated | | | 30 min, 1 h, 2 h, | |
| | Tabernanthalog | | | 4 h, 7 h, 24 h | |
| | (TBG) | | | | |
| 4 | Test Article or | PO | 10 | 15 min, 30 min, | 3 |
| | Deuterated | | | 45 min, 1 h, 2 h, | |
| | Tabernanthalog | | | 4 h, 7 h, 24 h | |
| | (TBG) | | | | |

### Dose Formulation Samples

Dose formulation samples are diluted in two steps with 50:50 (v/v) methanol/water to an appropriate concentration, then diluted 10:90 (v/v) with control matrix to match to the calibration standard in plasma.

### Sample Extraction Procedure

Calibration and QC standards, incurred samples, blank matrix and dose formulation samples are extracted by protein precipitation, via the addition of a bespoke acetonitrile (ACN)-based Internal Standard (IS) solution, containing several compounds and including Metoprolol and Rosuvastatin, both of which are monitored for during analysis. Following centrifugation, a 40 μL aliquot of supernatant is diluted by the addition of 80 μL water. The prepared sample extracts are analysed by LC-MS/MS.

### Example of Bioanalytical Method and Assay Information Document:

According to the plate layout, samples are aliquoted to wells in 0.8 mL 96-well plate (Abgene). This includes 30 μL for calibration, QC standards, blanks and dose formulation check. The calibration and QC standards are prepared according to the assay information. The dose formulation is diluted according to the assay information. The incurred samples are aliquoted according to the plate layout and assay information.

90 μL of ACN internal standard is added and vortex mixed for 5 minutes at 850 rpm. It is then centrifuged at nominally 4000 rpm for 10 minutes. 40 μL of supernatant is then transferred into a new 0.8 mL Abgene plate. 80 μL of water is added to all transferred supernatant followed by vortex mixing for 30 seconds at 1400 rpm. Samples are then analyzed immediately by LC-MS/MS or stored at +4° C. until analysis.

### Example 21—Biological Assays and Methods

### Agonist and Antagonist Profiles at Select 5-hydroxytryptamine (5-HT; Serotonin) Receptors

Protocol—in vitro testing at select 5-hydroxytryptamine (5-HT; serotonin) receptors was conducted.

### Assay Design: Calcium Mobilization

### Cell Handling

1. Cell lines were expanded from freezer stocks according to standard procedures.
2. Cells were seeded in a total volume of 20 μL into black-walled, clear-bottom, Poly-D-lysine coated 384-well microplates and incubated at 37° C. for the appropriate time prior to testing.

### Dye Loading

1. Assays were performed in 1×Dye Loading Buffer consisting of 1× Dye, 1× Additive A and 2.5 mM Probenecid in HBSS/20 mM Hepes. Probenicid was prepared fresh.
2. Cells were loaded with dye prior to testing. Media was aspirated from cells and replaced with 20 μL Dye Loading Buffer.
3. Cells were incubated for 30-60 minutes at 37° C.

### Agonist Format

1. For agonist determination, cells were incubated with sample to induce response.
2. After dye loading, cells were removed from the incubator and 10 μL HBSS/20 mM Hepes was added. 3× vehicle was included in the buffer when performing agonist dose curves to define
the EC80 for subsequent antagonist assays. Cells were incubated for 30 minutes at room temperature in the dark to equilibrate plate temperature.
3. Intermediate dilution of sample stocks was performed to generate 4× sample in assay buffer.
4. Compound agonist activity was measured on a FLIPR Tetra (MDS). Calcium mobilization was monitored for 2 minutes and 10 μL 4× sample in HBSS/20 mM Hepes was added to the cells 5 seconds into the assay.

### Allosteric Modulation Format

1. For allosteric determination, cells were pre-incubated with sample followed by agonist induction at the EC20 concentration.
2. Intermediate dilution of sample stocks was performed to generate 3× sample in assay buffer.
3. After dye loading, cells were removed from the incubator and 10 μL 3× sample was added. Cells were incubated for 30 minutes at room temperature in the dark to equilibrate plate temperature. Vehicle concentration was 1%.
4. Compound allosteric activity was measured on a FLIPR Tetra (MDS). Calcium mobilization was monitored for 2 minutes and 10 μL of 4× EC20 agonist in HBSS/20 mM Hepes was added to the cells 5 seconds into the assay.

### Antagonist Format

1. For antagonist determination, cells were pre-incubated with sample followed by agonist challenge at the EC80 concentration.
2. Intermediate dilution of sample stocks was performed to generate 3× sample in assay buffer.
3. After dye loading, cells were removed from the incubator and 10 L 3× sample was added. Cells were incubated for 30 minutes at room temperature in the dark to equilibrate plate temperature. Vehicle concentration was 1%.
4. Compound antagonist activity was measured on a FLIPR Tetra (MDS). Calcium mobilization was monitored for 2 minutes and 10 μL EC80 agonist in HBSS/20 mM Hepes was added to the cells 5 seconds into the assay.

### Data Analysis

1. Compound activity was analyzed using CBIS data analysis suite (ChemInnovation, CA).
2. For agonist mode assays, percentage activity is calculated using the following formula: % Activity=100%×(mean RFU of test sample−mean RFU of vehicle control)/(mean MAX RFU control ligand −mean RFU of vehicle control).
3. For positive allosteric mode assays, percentage modulation was calculated using the following formula: % Modulation=100%×((mean RFU of test sample−mean RFU of EC20 control)/(mean RFU of MAX control ligand −mean RFU of EC20 control)).
4. For antagonist and negative allosteric modulation mode assays, percentage inhibition is calculated using the following formula: % Inhibition=100%×(1−(mean RFU of test sample−mean RFU of vehicle control)/(mean RFU of EC80 control−mean RFU of vehicle control)).

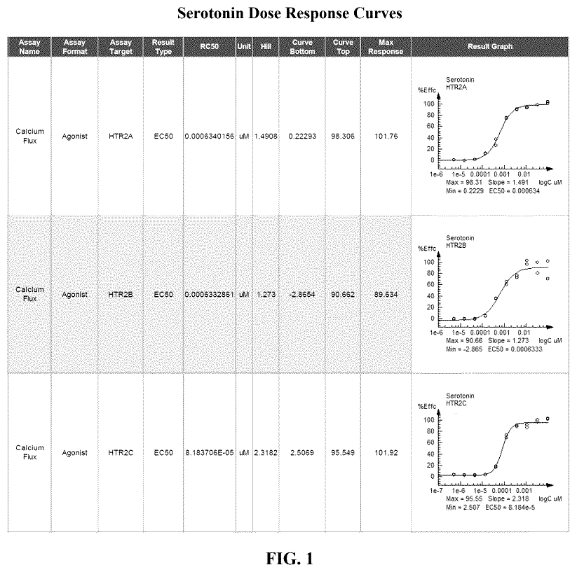

FIG. 1 shows control dose response curves for the selected GPCR Biosensor Assays.

TABLE 4
Activity of compounds against 5-HT2A, 5-HT2B and 5-HT2C receptors.

| | | 5-HT2A | 5-HT2B | 5-HT2C |
|---|---|---|---|---|
| | | agonist | agonist | antagonist |
| Compound | | Efficacy | Efficacy | inhibition |
| Number | Structure & Name | @ 10 mM | @ 5 mM | @10 mM |
| — | 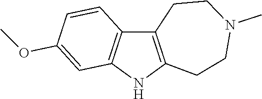 | 38.5% | −0.9% | 94.7% |
| | 8-methoxy-3-methyl-1,2,3,4,5,6- | | | |
| | hexahydroazepino[4,5-b]indole (TBG)- | | | |
| | literature reference (Nature 2021, 589, 474-479) | | | |
| 2 | 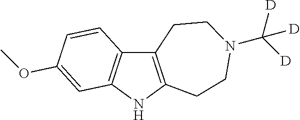 [SMILES: [2H]C([2H])([2H])N1CCc2[nH]c3cc(OC)ccc3c2CC1] | 38.0% | −0.4% | 94.5% |
| | 8-methoxy-3-trideuteromethyl-1,2,3,4,5,6- | | | |
| | hexahydroazepino[4,5-b]indole (compound 2) | | | |
| 1 | 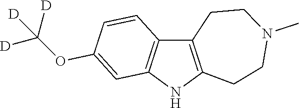 [SMILES: [2H]C([2H])([2H])Oc1ccc2c3c([nH]c2c1)CCN(C)CC3] | 30.4% | −8.3% | 95.5% |
| | 8-trideuteromethoxy-3-methyl-1,2,3,4,5,6- | | | |
| | hexahydroazepino[4,5-b]indole (compound 1) | | | |
| 3 | 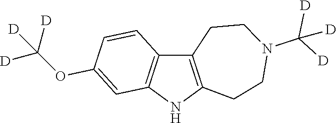 [SMILES: [2H]C([2H])([2H])Oc1ccc2c3c([nH]c2c1)CCN(C([2H])([2H])[2H])CC3] | 40.0% | −0.7% | 95.0% |
| | 8-trideuteromethoxy-3-trideuteromethyl- | | | |
| | 1,2,3,4,5,6-hexahydroazepino[4,5-b]indole | | | |
| | (compound 3) | | | |
| — | 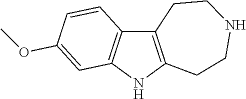 [SMILES: COc1ccc2c3c([nH]c2c1)CCNCC3] | 85.3% | 0.6% | 98.1% |
| | 8-methoxy-1,2,3,4,5,6-hexahydroazepino[4,5- | | | |
| | b]indole-literature reference (J. Med. Chem. | | | |
| | 1968, 11, 101-106) | | | |
| 163 | 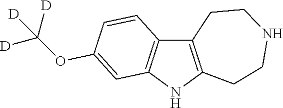 [SMILES: [2H]C([2H])([2H])Oc1ccc2c3c([nH]c2c1)CCNCC3] | 83.0% | −0.2% | 97.7% |
| | 8-trideuteromethoxy-1,2,3,4,5,6- | | | |
| | hexahydroazepino[4,5-b]indole (compound 163) | | | |
| 140 | 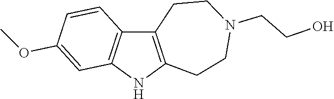 [SMILES: COc1ccc2c3c([nH]c2c1)CCN(CCO)CC3] | 48.1% | −0.9% | 93.8% |
| | 2-(8-methoxy-1,2,4,5-tetrahydroazepino[4,5- | | | |
| | b]indol-3(6H)-yl)ethanol (compound 140) | | | |
| 144 | 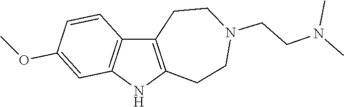 | −0.7% | −0.5% | 3.1% |
| | 2-(8-methoxy-1,2,4,5-tetrahydroazepino[4,5- | | | |
| | b]indol-3(6H)-yl)-N,N-dimethylethanamine | | | |
| | (compound 144) | | | |
| 143 | 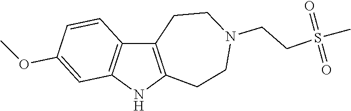 | 42.1% | −0.1% | 97.2% |
| | 8-methoxy-3-(2-(methylsulfonyl)ethyl)- | | | |
| | 1,2,3,4,5,6-hexahydroazepino[4,5-b]indole | | | |
| | (compound 143) | | | |
| 164 | 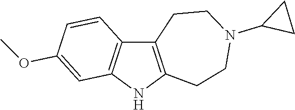 [SMILES: COc1ccc2c3c([nH]c2c1)CCN(C1CC1)CC3] | 61.0% | 2.7% | 100.1% |
| | 3-cyclopropyl-8-methoxy-1,2,3,4,5,6- | | | |
| | hexahydroazepino[4,5-b]indole (compound 164) | | | |
| 146 | 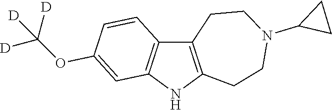 [SMILES: [2H]C([2H])([2H])Oc1ccc2c3c([nH]c2c1)CCN(C1CC1)CC3] | 69.4% | 9.0% | 103.0% |
| | 8-trideuteromethoxy-3-cyclopropyl-1,2,3,4,5,6- | | | |
| | hexahydroazepino[4,5-b]indole (compound 146) | | | |
| 138 | 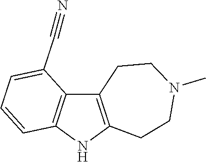 [SMILES: CN1CCc2[nH]c3cccc(C#N)c3c2CC1] | 15.4% | −0.4% | 100.2% |
| | 3-methyl-1,2,3,4,5,6-hexahydroazepino[4,5- | | | |
| | b]indole-10-carbonitrile (compound 138) | | | |
| 136 | 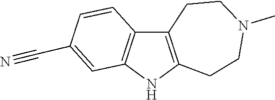 [SMILES: CN1CCc2[nH]c3cc(C#N)ccc3c2CC1] | 41.8% | −1.0% | 98.6% |
| | 3-methyl-1,2,3,4,5,6-hexahydroazepino[4,5- | | | |
| | b]indole-8-carbonitrile (compound 136) | | | |
| 110 | 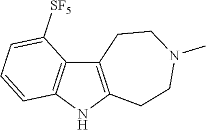 | 8.9% | 4.0% | 102.6% |
| | pentafluoro-(3-methyl-2,4,5,6-tetrahydro-1H- | | | |
| | azepino[4,5-b]indol-10-yl)-λ⁶-sulfane | | | |
| | (compound 110) | | | |
| 109 | 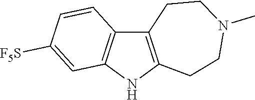 | 13.7% | 8.5% | 101.3% |
| | pentafluoro-(3 -methyl-2,4,5,6-tetrahy dro-1H- | | | |
| | azepino[4,5-b]indol-8-yl)-λ⁶-sulfane (compound | | | |
| | 109) | | | |
| 165 | 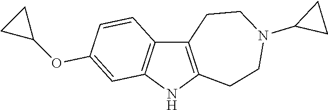 [SMILES: c1cc2c3c([nH]c2cc1OC1CC1)CCN(C1CC1)CC3] | −2.7% | 0.9% | 71.7% |
| | 8-cyclopropoxy-3-cyclopropyl-1,2,3,4,5,6- | | | |
| | hexahydroazepino[4,5-b]indole (compound 165) | | | |
| 117 | 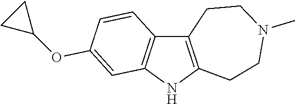 [SMILES: CN1CCc2[nH]c3cc(OC4CC4)ccc3c2CC1] | 10.4% | 1.7% | 99.7% |
| | 8-cyclopropoxy-3-methyl-1,2,3,4,5,6- | | | |
| | hexahydroazepino[4,5-b]indole (compound 117) | | | |
| 145 | 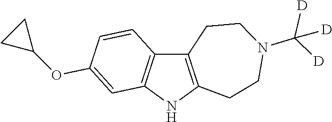 [SMILES: [2H]C([2H])([2H])N1CCc2[nH]c3cc(OC4CC4)ccc3c2CC1] | 5.8% | 1.5% | 101.2% |
| | 8-cyclopropoxy-3-trideuteromethyl-1,2,3,4,5,6- | | | |
| | hexahydroazepino[4,5-b]indole (compound 145) | | | |
| 124 | 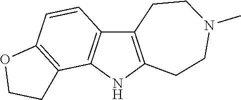 [SMILES: CN1CCc2[nH]c3c4c(ccc3c2CC1)OCC4] | 83.8% | −0.7% | 93.1% |
| | 8-methyl-1,2,6,7,8,9,10,11- | | | |
| | octahydroazepino[4,5-b]furo[2,3-g]indole | | | |
| | (compound 124) | | | |
| 166 | 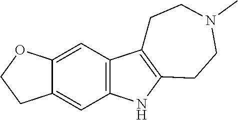 [SMILES: CN1CCc2[nH]c3cc4c(cc3c2CC1)OCC4] | 53.5% | 0.3% | 97.9% |
| | 8-methyl-2,3,5,6,7,8,9,10-octahydroazepino[4,5- | | | |
| | b]furo[2,3-findole (compound 166) | | | |
| 122 | 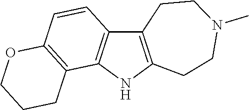 [SMILES: CN1CCc2[nH]c3c4c(ccc3c2CC1)OCCC4] | 54.0% | −0.8% | 98.9% |
| | 9-methyl-2,3,7,8,9,10,11,12-octahydro-1H- | | | |
| | azepino[4,5-b]pyrano[2,3-g]indole | | | |
| | (compound 122) | | | |
| — | SB242084 | — | — | 100.6% |

A compound was deemed to have an advantageous property if it was found to be an agonist, or partial agonist, of the 5-HT_{2A} receptor, when screened at a concentration of 10 μM (ten micromolar), whilst also not serving as an agonist of the 5-HT2B receptor (defined as <20% relative efficacy in relation to 5-HT) at a screening concentration of 5 μM (five micromolar). As discussed above, agonism, or partial agonism, of the 5-HT_{2A} receptor (also known as positive allosteric modulation, or 5-HT_{2A} modulation) is useful for the treatment of neurological and psychiatric disorders. For example, 5-HT_{2A} agonism has been correlated with the promotion of neural plasticity (Ly et al., 2018). Agonism of the 5-HT2B receptor has been associated with unwanted cardiac valvulopathy side-effects, a form of cardio-toxicity (Rothman et al., Circulation. 2000, 102, 2836-2841; Fitzgerald et al., Molecular Pharmacology 2000, 57, 75-81). A compound was also deemed to have an advantageous property if it was found to be an antagonist of the 5-HT2C receptor, when screened at a concentration of 10 μM (ten micromolar). Antagonists of the 5-HT2C receptor are also useful for the treatment of neurological and psychiatric disorders (Kennett et al., Neuropharmacology 1997, 36, 609-620).

The compounds disclosed herein serve as agonists, or partial agonists, of the 5-HT_{2A} receptor at 10 μM (ten micromolar), and do not serve as agonists of the 5-HT2B receptor at 5 μM (five micromolar). For research comparative purposes, two literature compounds—Tabernanthalog (TBG) (Nature 2021, 589, 474-479) and 8-methoxy-1,2,3,4,5,6-hexahydroazepino[4,5-b]indole (J. Med. Chem. 1968, 11, 101-106)—were included in the screening assays. Additionally, 5-hydroxytryptamine/serotonin (5-HT) was included in the screening assay as a positive control agonist. Compounds disclosed herein have advantageous properties at the 5-HT_{2A} or 5-HT2B receptor as discussed herein and include: 8-methoxy-3-trideuteromethyl-1,2,3,4,5,6-hexahydroazepino[4,5-b]indole (compound 2) 8-trideuteromethoxy-3-methyl-1,2,3,4,5,6-hexahydroazepino[4,5-b]indole (compound 1) 8-trideuteromethoxy-3-trideuteromethyl-1,2,3,4,5,6-hexahydroazepino[4,5-b]indole (compound 3) 8-trideuteromethoxy-1,2,3,4,5,6-hexahydroazepino[4,5-b]indole (compound 163) 2-(8-methoxy-1,2,4,5-tetrahydroazepino[4,5-b]indol-3(6H)-yl)ethanol (compound 140) 8-methoxy-3-(2-(methylsulfonyl)ethyl)-1,2,3,4,5,6-hexahydroazepino[4,5-b]indole (compound 143) 3-cyclopropyl-8-methoxy-1,2,3,4,5,6-hexahydroazepino[4,5-b]indole (compound 164) 8-trideuteromethoxy-3-cyclopropyl-1,2,3,4,5,6-hexahydroazepino[4,5-b]indole (compound 146) 3-methyl-1,2,3,4,5,6-hexahydroazepino[4,5-b]indole-10-carbonitrile (compound 138) 3-methyl-1,2,3,4,5,6-hexahydroazepino[4,5-b]indole-8-carbonitrile (compound 136) pentafluoro-(3-methyl-2,4,5,6-tetrahydro-1H-azepino[4,5-b]indol-10-yl)-λ⁶-sulfane (compound 110) pentafluoro-(3-methyl-2,4,5,6-tetrahydro-1H-azepino[4,5-b]indol-8-yl)-λ⁶-sulfane (compound 109) 8-cyclopropoxy-3-methyl-1,2,3,4,5,6-hexahydroazepino[4,5-b]indole (compound 117) 8-cyclopropoxy-3-trideuteromethyl-1,2,3,4,5,6-hexahydroazepino[4,5-b]indole (compound 145) 8-methyl-1,2,6,7,8,9,10,11-octahydroazepino[4,5-b]furo[2,3-g]indole (compound 124) 8-methyl-2,3,5,6,7,8,9,10-octahydroazepino[4,5-b]furo[2,3-J]indole (compound 166) 9-methyl-2,3,7,8,9,10,11,12-octahydro-1H-azepino[4,5-b]pyrano[2,3-g]indole (compound 122)

In some embodiments, the compounds of the present disclosure are selected from, 8-methoxy-3-trideuteromethyl-1,2,3,4,5,6-hexahydroazepino[4,5-b]indole (compound 2) 8-trideuteromethoxy-3-methyl-1,2,3,4,5,6-hexahydroazepino[4,5-b]indole (compound 1) 8-trideuteromethoxy-3-trideuteromethyl-1,2,3,4,5,6-hexahydroazepino[4,5-b]indole (compound 3) 2-(8-methoxy-1,2,4,5-tetrahydroazepino[4,5-b]indol-3(6H)-yl)ethanol (compound 140) 8-methoxy-3-(2-(methylsulfonyl)ethyl)-1,2,3,4,5,6-hexahydroazepino[4,5-b]indole (compound 143) 3-methyl-1,2,3,4,5,6-hexahydroazepino[4,5-b]indole-8-carbonitrile (compound 136) 8-methyl-2,3,5,6,7,8,9,10-octahydroazepino[4,5-b]furo[2,3-J]indole (compound 166) 9-methyl-2,3,7,8,9,10,11,12-octahydro-1H-azepino[4,5-b]pyrano[2,3-g]indole (compound 122)

Compounds may also serve as antagonists of the 5-HT2C receptor at 1 μM (ten micromolar). For research comparative purposes, two literature compounds—Tabernanthalog (TBG) (Nature 2021, 589, 474-479) and 8-methoxy-1,2,3,4,5,6-hexahydroazepino[4,5-b]indole (J. Med. Chem. 1968, 11, 101-106)—were included in the screening assays. Additionally, 5-hydroxytryptamine/serotonin (5-HT) was included in the screening assay as a positive control agonist, and SB242084 was used in the screening assay as a literature 5-HT2C antagonist positive control. Compounds disclosed herein have advantageous properties at the 5-HT2C receptor as discussed herein. In some embodiments, compounds disclosed herein have advantageous properties at the 5-HT2C receptor and include, 8-methoxy-3-trideuteromethyl-1,2,3,4,5,6-hexahydroazepino[4,5-b]indole (compound 2) 8-trideuteromethoxy-3-methyl-1,2,3,4,5,6-hexahydroazepino[4,5-b]indole (compound 1) 8-trideuteromethoxy-3-trideuteromethyl-1,2,3,4,5,6-hexahydroazepino[4,5-b]indole (compound 3) 8-trideuteromethoxy-1,2,3,4,5,6-hexahydroazepino[4,5-b]indole (compound 163) 2-(8-methoxy-1,2,4,5-tetrahydroazepino[4,5-b]indol-3(6H)-yl)ethanol (compound 140) 8-methoxy-3-(2-(methylsulfonyl)ethyl)-1,2,3,4,5,6-hexahydroazepino[4,5-b]indole (compound 143) 3-cyclopropyl-8-methoxy-1,2,3,4,5,6-hexahydroazepino[4,5-b]indole (compound 164) 8-trideuteromethoxy-3-cyclopropyl-1,2,3,4,5,6-hexahydroazepino[4,5-b]indole (compound 146) 3-methyl-1,2,3,4,5,6-hexahydroazepino[4,5-b]indole-10-carbonitrile (compound 138) 3-methyl-1,2,3,4,5,6-hexahydroazepino[4,5-b]indole-8-carbonitrile (compound 136) pentafluoro-(3-methyl-2,4,5,6-tetrahydro-1H-azepino[4,5-b]indol-10-yl)-λ⁶-sulfane (compound 110) pentafluoro-(3-methyl-2,4,5,6-tetrahydro-1H-azepino[4,5-b]indol-8-yl)-λ⁶-sulfane (compound 109) 8-cyclopropoxy-3-cyclopropyl-1,2,3,4,5,6-hexahydroazepino[4,5-b]indole (compound 165) 8-cyclopropoxy-3-methyl-1,2,3,4,5,6-hexahydroazepino[4,5-b]indole (compound 117) 8-cyclopropoxy-3-trideuteromethyl-1,2,3,4,5,6-hexahydroazepino[4,5-b]indole (compound 145) 8-methyl-1,2,6,7,8,9,10,11-octahydroazepino[4,5-b]furo[2,3-g]indole (compound 124) 8-methyl-2,3,5,6,7,8,9,10-octahydroazepino[4,5-b]furo[2,3-f]indole (compound 166) 9-methyl-2,3,7,8,9,10,11,12-octahydro-1H-azepino[4,5-b]pyrano[2,3-g]indole (compound 122)

Head-Twitch Response (HTR). The head-twitch response assay is performed as is known to those of skill in the art using both male and female C57BL/6J mice (3 per treatment). The mice are obtained and are approximately 8 weeks old at the time of the experiments. Compounds are administered via intraperitoneal injection (5 mL/kg) using 0.9% saline as the vehicle. As a positive control, Psilocybin was used. Behavior is videotaped, later scored by two blinded observers, and the results are averaged (Pearson correlation coefficient=0.93).

### Background: Hallucinogen-Induced Head Shakes and Twitches

Mice administered LSD were reported by Keller and Umbreit (1956) to respond with rapid and violent head shaking that does not occur in normal mice. This response was found to be remarkably consistent when scored by different observers across laboratories. The head-shake response is elicited by a wide variety of known hallucinogens such as LSD, psilocybin, psilocin, N,N-dimethyltryptamine (DMT), and mescaline as well as serotonin-releasing agents and direct 5-HT2 agonists (Canal and Morgan 2012). 2,5-dimethoxy-4-iodoamphetamine (DOI) has also been reported to elicit head-shakes in rats (Arnt and Hyttel 1989, Kennett et al., 1994) and head-twitches in mice Darmani et al., 1990), both of which were blocked by administration of the fairly selective 5-HT_{2A} antagonist ketanserin. Later studies have confirmed 5-HT_{2A} receptors are the primary, direct mediators of the response and that the headshake response in rats is essentially the same as head-twitches in mice, at least in regards to similarity in appearance and 5-HT_{2A} receptor dependence (Canal and Morgan 2012). The head twitch and head shake response in mice and rats have therefore been widely used to explore the effect of treatments on 5-HT_{2A} receptors in vivo.

### REFERENCES

Arnt J, Hyttel J. (1989). Facilitation of 8-OHDPAT-induced forepaw treading of rats by the 5-HT2 agonist DOI. Eur. J. Pharmacol., 161:45.
Canal C E., Morgan D. (2012). Head-twitch response in rodents induced by the hallucinogen 2,5-dimethoxy-4-iodoamphetamine: a comprehensive history, a re-evaluation of mechanisms and its utility as a model. Drug Test Anal. 4, 556-576.
Darmani N A, Martin B R, Pandey U, Glennon R A. (1990). Do functional relationships exist between 5-HT1A and 5-HT2 receptors?Pharmacol Biochem Behav., 36: 901-6.
Keller D L, Umbreit W W. (1956). Permanent alteration of behavior in mice by chemical and psychological means. Science, 124: 723.
Kennett G A, Wood M D, Glen A, Grewal S, Forbes I, Gadre A, Blackburn T P. (1994). In vivo properties of SB 200646A, a 5-HT2C/2B receptor antagonist. Br J Pharmacol. 111: 797-802.

### Protocol 1: Effect of TBG, Five Representative Compounds of the Application, and Psilocybin on Head Twitches in Mice.

### Animals

24 Male C57BL/6J mice (approximately 25 g) were group housed in a stock room. Animals were maintained under a 12 h light/dark cycle, at 23° C. with humidity controlled according to Home Office regulations.

### Formulation

Compound 117, compound 109, compound 122, compound 143, compound 136 and TBG (all compounds supplied as free bases) were each formulated in DMSO:saline [10:90] at a concentration of 2 mg/mL to give a dose of 10 mg/kg when administered ip in a 5 mL/kg dosing volume.

Psilocybin (free base) was formulated in DMSO:saline [10:90] at a concentration of 0.4 mg/mL to give a dose of 2 mg/kg when administered ip in a 5 mL/kg dosing volume.

### Procedure

At T=−60 mi, C57BL/6J mice were individually housed into transparent observation cages with bedding removed and left to habituate.

At T=0 h, groups of 3 mice were dosed intraperitoneally with either Vehicle or compound 117 or compound 109 or compound 122 or compound 143 or compound 136 or TBG each at 10 mg/kg or psilocybin at 2 mg/kg. Following dosing, mice were replaced into the observation cages and head twitch behavior was monitored for 40 min after agonist dosing.

TABLE 5
Synopsis of mouse head twitch test schedule

| | 60 min | | T = 0-40 min |
|---|---|---|---|
| | pre-test | | Assess no of |
| | Place in test | T = 0 pre test | head twitches |
| Grp | cage to | Treatment IP | 40 min per |
| (n) | habituate | (5 mL/kg DMSO: saline) | mouse |
| 3 | Yes | Vehicle | Yes |
| 3 | Yes | Compound 117 (8-cyclopropoxy-3- | Yes |
| | | methyl-1,2,3,4,5,6-hexahydroazepino[4,5- | |
| | | b]indole) 10 mg/kg | |
| 3 | Yes | Compound 109 (pentafluoro-(3-methyl- | Yes |
| | | 2,4,5,6-tetrahydro-1H-azepino[4,5-b]indol- | |
| | | 8-yl)-λ⁶-sulfane) 10 mg/kg | |
| 3 | Yes | Compound 122 (9-methyl- | Yes |
| | | 2,3,7,8,9,10,11,12-octahydro-1H- | |
| | | azepino[4,5-b]pyrano[2,3-g]indole) 10 | |
| | | mg/kg | |
| 3 | Yes | Compound 143 (8-methoxy-3-(2- | Yes |
| | | (methylsulfonyl)ethyl)-1,2,3,4,5,6- | |
| | | hexahydroazepino[4,5-b]indole) 10 mg/kg | |
| 3 | Yes | Compound 136 (3-methyl-1,2,3,4,5,6- | Yes |
| | | hexahydroazepino[4,5-b]indole-8- | |
| | | carbonitrile) * | |
| 3 | Yes | TBG (8-methoxy-3-methyl-1,2,3,4,5,6- | Yes |
| | | hexahydroazepino[4,5-b]indole) 10 mg/kg | |
| 3 | Yes | Psilocybin 2 mg/kg | Yes |

* Study of compound 136was conducted on a separate day.

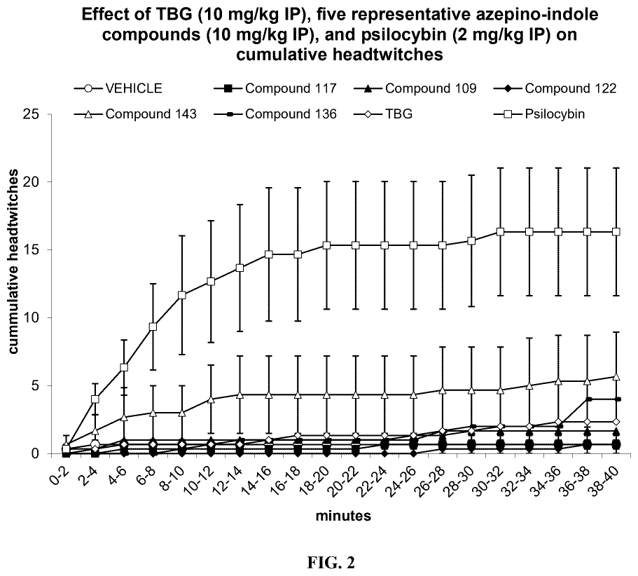

FIG. 2 provides a graph showing average cumulative head twitches induced by tabernanthalog, five representative compounds of the application, and psilocybin.

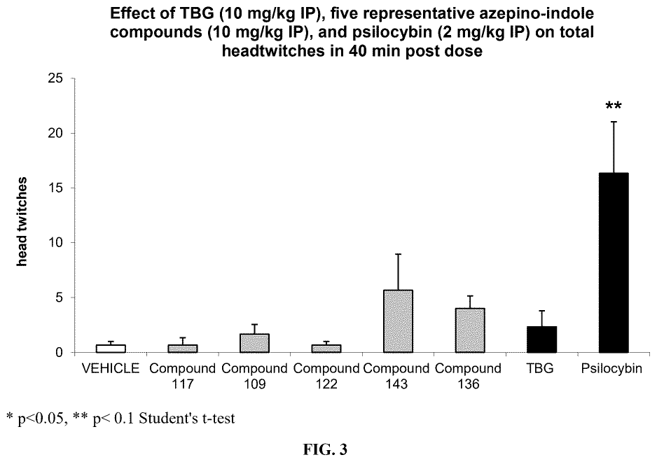

FIG. 3 provides a bar chart showing total average head twitches induced by tabernanthalog, the five representative compounds of the application, and psilocybin in the 40 minutes post-dose.

As illustrated in FIGS. 2 and 3, the representative compounds of the application did not produce a significant head-twitch response compared to placebo. Only psilocybin produced significant increases in head-twitch. This experiment shows that these compounds are not expected to produce hallucinations in humans. Hallucinations are a treatment limiting side effect and the lack of hallucinatory activity shows that these compounds have advantages over psychedelics such as ibogaine that cause treatment limiting hallucinations.

Serotonin and Opioid Receptor Functional Assays. Functional assay screens at 5-HT and opioid receptors are performed in parallel using the same compound dilutions and 384-well format high-throughput assay platforms. Assays assess activity at all human isoforms of the receptors, except where noted for the mouse 5-HT_{2A} receptor. Receptor constructs in pcDNA vectors are generated from the Presto-Tango GPCR library with minor modifications. All compounds are serially diluted in drug buffer (HBSS, 20 mM HEPES, pH 7.4 supplemented with 0.1% bovine serum albumin and 0.01% ascorbic acid) and dispensed into 384-well assay plates using a FLIPR^{TETRA} (Molecular Devices). Every plate included a positive control such as 5-HT (for all 5-HT receptors), DADLE (DOR), salvinorin A (KOR), and DAMGO (MOR). For measurements of 5-HT_{2A}, 5-HT2B, and 5-HT2C Gq-mediated calcium flux function, HEK Flp-In 293 T-Rex stable cell lines (Invitrogen) are loaded with Fluo-4 dye for one hour, stimulated with compounds and read for baseline (0-10 seconds) and peak fold-over-basal fluorescence (5 minutes) at 25° C. on the FLIPR^{TETRA}. For measurement of 5-HT6 and 5-HT7a functional assays, Gs-mediated cAMP accumulation is detected using the split-luciferase GloSensor assay in HEKT cells measuring luminescence on a Microbeta Trilux (Perkin Elmer) with a 15 min drug incubation at 25° C. For 5-HT1A, 5-HT1B, 5-HT1F, MOR, KOR, and DOR functional assays, Gi/o-mediated cAMP inhibition is measured using the split-luciferase GloSensor assay in HEKT cells, conducted similarly as above, but in combination with either 0.3 μM isoproterenol (5-HT1A, 5-HT1B, 5-HT1F) or 1 μM forskolin (MOR, KOR, and DOR) to stimulate endogenous cAMP accumulation. For measurement of 5-HT1D, 5-HT1E, 5-HT4, and 5-HT5A functional assays, P-arrestin2 recruitment is measured by the Tango assay utilizing HTLA cells expressing TEV fused-P-arrestin2, as described previously with minor modifications. Data for all assays are plotted and non-linear regression is performed using “log(agonist) vs. response” in Graphpad Prism to yield Emax and EC₅₀ parameter estimates.

5HT_{2A} Sensor Assays. HEK293T (ATCC) 5HT_{2A} sensor stable line (sLight1.3s) is generated via lentiviral transduction of HIV-EF1α-sLight1.3 and propagated from a single colony. Lentivirus is produced using 2^{nd} generation lentiviral plasmids pHIV-EF1α-sLight1.3, pHCMV-G, and pCMV-deltaR8.2.

For the screening of the compounds, sLight1.3s cells are plated in 96-well plates at a density of 40000 24-hours prior to imaging. On the day of imaging, compounds solubilized in DMSO are diluted from the 100 mM stock solution to working concentrations of 1 mM, 100 mM and 1 μM with a DMSO concentration of 1%. Immediately prior to imaging, cells growing in DMEM (Gibco) are washed 2× with HBSS (Gibco) and in agonist mode 180 μL of HBSS or in antagonist mode 160 μL of HBSS is added to each well after the final wash. For agonist mode, images are taken before and after the addition of the 20 μL compound working solution into the wells containing 180 μL HBSS. This produces final compound concentrations of 100 mM, 10 mM and 100 nM with a DMSO concentration of 0.1%. For antagonist mode, images are taken before and after addition of 20 μL of 900 nM 5-HT and again after 20 μL of the compound working solutions to produce final concentrations of 100 nM for 5HT and 100 mM, 10 mM and 100 nM for the compounds with a DMSO concentration of 0.1%. Each compound is tested in triplicate (3 wells) for each concentration (100 mM, 10 mM and 100 nM). Additionally, within each plate, 100 nM 5HT and 0.1% DMSO controls are also imaged.

Imaging is performed using the Leica DMi8 inverted microscope with a 40× objective using the FITC preset with an excitation of 460 nm and emission of 512-542 nm. For each well, the cellular membrane where the 5HT_{2A} sensor is targeted is autofocused using the adaptive focus controls and 5 images from different regions within the well are taken with each image processed from a 2×2 binning.

For data processing, the membranes from each image are segmented and analyzed using a custom algorithm written in MATFAB producing a single raw fluorescence intensity value. For each well the 5 raw fluorescence intensity values generated from the 5 images are averaged and the change in fluorescence intensity (dFF) is calculated as:

$$dFF = ( {F}_{sat} - {F}_{apo} ) / {F}_{apo}$$

For both agonist and antagonist modes, the fluorescence intensity values before compound addition in FIBSS only are used as the Fₐₚₒ values while the fluorescence intensity values after compound addition are used as the Fₛₐₜ values.

For agonist mode, data are as percent activation relative to 5HT, where 0 is the average of the DMSO wells and 100 is the average of the 100 μM 5HT wells. For antagonist mode, the inactivation score is calculated as:

$$Inactivation score = ( dFFF ( Compound + 5 HT ) - dFF ( 5 HT ) ) / dFF ( 5 HT )$$

### Example 22—Plasticity Effects

Treatment of rat embryonic cortical neurons with compounds of Formulas I, I-1, I-1A, I-1B, II, IIa, IIb, IIc, IId, IIe, IIf, IIg, Table 1, or a pharmaceutically acceptable salt thereof is evaluated for increased dendritic arbor complexity at 6 days in vitro (DIV6) as measured by Sholl analysis. The effect of the present compounds on dendritic growth can be determined to be 5-HT_{2A}-dependent, if pretreatment with ketanserin—a 5-HT_{2A} antagonist—inhibits their effects.

In addition to promoting dendritic growth, the present compounds also are evaluated for increased dendritic spine density to a comparable extent as ibogaine in mature cortical cultures (DIV20). The effect of the compounds on cortical dendritic spine dynamics in vivo using transcranial 2-photon imaging is assessed. First, spines are imaged on specific dendritic loci defined by their relation to blood vessel and dendritic architectures. Next, the animals are systemically administered vehicle, a compound of the present invention, or the hallucinogenic 5-HT_{2A} agonist 2,5-dimethoxy-4-iodoamphetamine (DOI). After 24 h, the same dendritic segments are re-imaged, and the number of spines gained or lost is quantified. Examples of the presently disclosed compounds increase spine formation in mouse primary sensory cortex, suggesting that the present compounds support neuronal plasticity.

As increased cortical structural plasticity in the anterior parts of the brain mediates the sustained (>24 h) antidepressant-like effects of ketamine and play a role in the therapeutic effects of 5-HT_{2A} agonists, the impact of the present compounds on forced swim test (FST) behavior is evaluated. First, a pretest is used to induce a depressive phenotype. Compounds are administered 24 h after the pre-test, and the FST is performed 24 h and 7 d post compound administration. Effective compounds of the invention, like ketamine, significantly reduced immobility 24 h after administration.

Example 23—Dendritogenesis Assays. Compounds disclosed herein are evaluated for their ability to increase dendritic arbor complexity in cultures of cortical neurons using a phenotypic assay. Following treatment, neurons are fixed and visualized using an antibody against MAP2—a cytoskeletal protein localized to the somatodendritic compartment of neurons. Sholl analysis is then performed, and the maximum number of crossings (Nₘₐₓ) is used as a quantitative metric of dendritic arbor complexity. For statistical comparisons between specific compounds, the raw Nₘₐₓ values are compared. Percent efficacies are determined by setting the Nₘₐₓ values for the vehicle (DMSO) and positive (ketamine) controls equal to 0% and 100%, respectively.

Animals. For the dendritogenesis experiments, timed pregnant Sprague Dawley rats are obtained. For the head-twitch response assay, male and female C57BL/6J mice are obtained.

Example 24—Dendritogenesis —Sholl Analysis. Dendritogenesis experiments are performed following a previously published methods with slight modifications. Neurons are plated in 96-well format (200 μL of media per well) at a density of approximately 15,000 cells/well in Neurobasal (Life Technologies) containing 1% penicillin-streptomycin, 10% heat-inactivated fetal bovine serum, and 0.5 mM glutamine. After 24 h, the medium is replaced with Neurobasal containing 1×B27 supplement (Life Technologies), 1% penicillin-streptomycin, 0.5 mM glutamine, and 12.5 μM glutamate. After 3 days in vitro (DIV3), the cells are treated with compounds. All compounds tested in the dendritogenesis assays are treated at 10 μM. Stock solutions of the compounds in DMSO are first diluted 100-fold in Neurobasal before an additional 10-fold dilution into each well (total dilution=1:1000; 0.1% DMSO concentration). Treatments are randomized. After 1 h, the media is removed and replaced with new Neurobasal media containing 1×B27 supplement, 1% penicillin-streptomycin, 0.5 mM glutamine, and 12.5 mM glutamate. The cells are allowed to grow for an additional 71 h. At that time, neurons are fixed by removing 80% of the media and replacing it with a volume of 4% aqueous paraformaldehyde (Alfa Aesar) equal to 50% of the working volume of the well. Then, the cells are incubated at room temperature for 20 min before the fixative is aspirated and each well washed twice with DPBS. Cells are permeabilized using 0.2% Triton X-100 (ThermoFisher) in DPBS for 20 minutes at room temperature without shaking. Plates are blocked with antibody diluting buffer (ADB) containing 2% bovine serum albumin (BSA) in DPBS for 1 h at room temperature. Then, plates are incubated overnight at 4° C. with gentle shaking in ADB containing a chicken anti-MAP2 antibody (1:10,000; EnCor, CPCA-MAP2). The next day, plates are washed three times with DPBS and once with 2% ADB in DPBS. Plates are incubated for 1 h at room temperature in ADB containing an anti-chicken IgG secondary antibody conjugated to Alexa Fluor 488 (Life Technologies, 1:500) and washed five times with DPBS. After the final wash, 100 μL of DPBS is added per well and imaged on an ImageXpress Micro XL High-Content Screening System (Molecular Devices, Sunnyvale, CA) with a 20× objective. Images are analyzed using ImageJ Fiji (version 1.51 W). First, images corresponding to each treatment are sorted into individual folders that are then blinded for data analysis. Plate controls (both positive and negative) are used to ensure that the assay is working properly as well as to visually determine appropriate numerical values for brightness/contrast and thresholding to be applied universally to the remainder of the randomized images. Next, the brightness/contrast settings are applied, and approximately 1-2 individual pyramidal-like neurons per image (i.e., no bipolar neurons) are selected using the rectangular selection tool and saved as separate files. Neurons are selected that do not overlap extensively with other cells or extend far beyond the field of view.

Example 25—In Vivo Spine Dynamics. Male and female Thyl-GFP-M line mice (n=5 per condition) are purchased from The Jackson Laboratory (JAX #007788) and maintained. In vivo transcranial two-photon imaging and data analysis are performed as previously described. Briefly, mice are anesthetized with an intraperitoneal (i.p.) injection of a mixture of ketamine (87 mg/kg) and xylazine (8.7 mg/kg). A small region of the exposed skull is manually thinned down to 20-30 pm for optical access. Spines on apical dendrites in mouse primary sensory cortices are imaged using a Bruker Ultima IV two-photon microscope equipped with an Olympus water-immersion objective (40×, NA=0.8) and a Ti:Sapphire laser (Spectra-Physics Mai-Tai, excitation wavelength 920 nm). Images are taken at a zoom of 4.0 (pixel size 0.143×0.143 pm) and Z-step size of 0.7 pm. The mice receive an i.p. injection (injection volume=5 mL/kg) of DOI (10 mg/kg) or TBG (50 mg/kg) immediately after they recover from anesthesia given prior to the first imaging session. The animals are re-imaged 24 h after drug administration. Dendritic spine dynamics are analyzed using ImageJ. Spine formation and elimination are quantified as percentages of spine number on day 0.

Example 26—Forced Swim Test (FST). Male C57/BL6J mice (9-10 weeks old at time of experiment) are obtained. After 1 week in the vivarium each mouse is handled for approximately 1 minute by the experimenter for 3 consecutive days leading up to the first FST. All experiments are carried out by the same experimenter who performs handling. During the FST, mice undergo a 6 min swim session in a clear Plexiglas cylinder 40 cm tall, 20 cm in diameter, and filled with 30 cm of 24±1° C. water. Fresh water is used for every mouse. After handling and habituation to the experimenter, drug-naive mice first undergo a pretest swim to more reliably induce a depressive phenotype in the subsequent FST sessions. Immobility scores for all mice are determined after the pre-test and mice are randomly assigned to treatment groups to generate groups with similar average immobility scores to be used for the following two FST sessions. The next day, the animals receive intraperitoneal injections of experimental compounds (20 mg/kg), a positive control (ketamine, 3 mg/kg), or vehicle (saline). The animals are subjected to the FST 30 mins after injection and then returned to their home cages. All FSTs are performed between the hours of 8 am and 1 pm. Experiments are video-recorded and manually scored offline. Immobility time—defined as passive floating or remaining motionless with no activity other than that needed to keep the mouse's head above water—is scored for the last 4 min of the 6 min trial.

Statistical analysis. Treatments are randomized, and data are analyzed by experimenters blinded to treatment conditions. Statistical analyses are performed using GraphPad Prism (version 8.1.2). The specific tests are F-statistics and degrees of freedom. All comparisons are planned prior to performing each experiment. For dendritogenesis experiments a one-way ANOVA with Dunnett's post hoc test is deemed most appropriate. Ketamine is included as a positive control to ensure that the assay is working properly.

To assess the anti-addictive potential of the present compounds, an alcohol drinking paradigm that models heavy alcohol use and binge drinking behavior in humans is employed. Using a 2-bottle choice setup (20% ethanol (v/v), EtOH vs. water, H₂O), mice are subjected to repeated cycles of binge drinking and withdrawal over the course of 7 weeks.

This schedule results in heavy EtOH consumption, binge drinking-like behavior, and generates blood alcohol content equivalent to that of human subjects suffering from alcohol use disorder (AUD). Next, compounds of the invention are administered via intraperitoneal injection 3 h prior to a drinking session, and EtOH and H₂O consumption is monitored. Effective compounds of the invention robustly reduce binge drinking during the first 4 h, decreasing EtOH consumption. With exemplary compounds, consumption of ethanol is lower for at least two days following administration with no effect on water intake. Efficacy in this assay suggests the present compounds are useful for the treatment of AUD.

In view of the many possible embodiments to which the principles of the disclosed invention may be applied, it should be recognized that the illustrated embodiments are only examples of the invention and should not be taken as limiting the scope of the invention. Rather, the scope of the invention is defined by the following claims. We therefore claim as our invention all that comes within the scope and spirit of these claims.

1.-102. (canceled)

103. A compound of Formula (I-1) 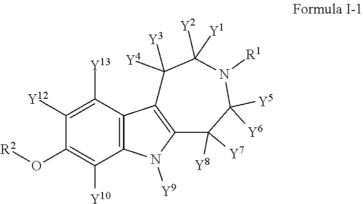
or a pharmaceutically acceptable salt thereof; wherein:
R¹ is CH₃, CH₂D, CHD₂ or CD₃;
Y¹, Y², Y³, Y⁴, Y⁵, Y⁶, Y⁷, Y⁸, Y⁹, Y¹⁰, Y¹², and Y¹³ are each independently selected from H or D;
R² is CH₃, CH₂D, CHD₂ or CD₃; and
at least one of R¹ and R² comprise deuterium.

104. The compound of claim 103, wherein R¹ is CH₂D, CHD₂ or CD₃.

105. The compound of claim 104, wherein R¹ is CD₃.

106. The compound of claim 103, wherein R² is CH₂D, CHD₂ or CD₃.

107. The compound of claim 106, wherein R² is CD₃.

108. The compound of claim 104, wherein R² is CH₂D, CHD₂ or CD₃.

109. The compound of claim 104, wherein R² is CD₃.

110. The compound of claim 105, wherein R² is CD₃.

111. The compound of claim 103, wherein at least one of Y¹, Y², Y³, Y⁴, Y⁵, Y⁶, Y⁷, Y⁸, Y⁹, Y¹⁰, Y¹¹, Y¹², and Y¹³ is D.

112. The compound of claim 103, wherein at least one of Y¹, Y², Y³, Y⁴, Y⁵, Y⁶, Y⁷ and Y⁸ is D.

113. The compound of claim 103, wherein at least one of Y¹⁰, Y¹², and Y¹³ is D.

114. The compound of claim 103, wherein the compound is 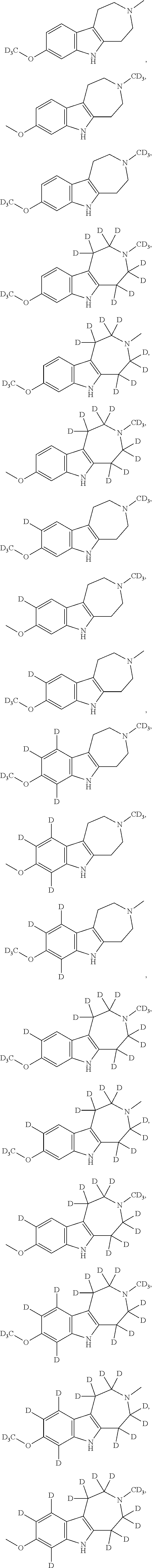 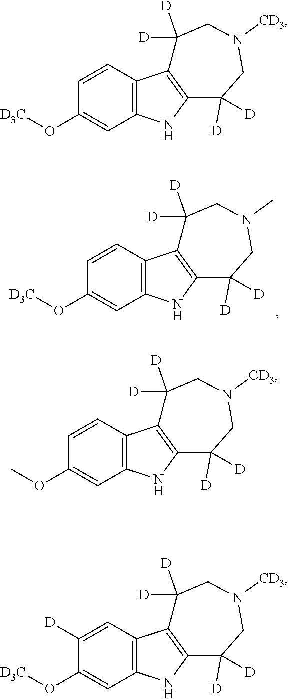 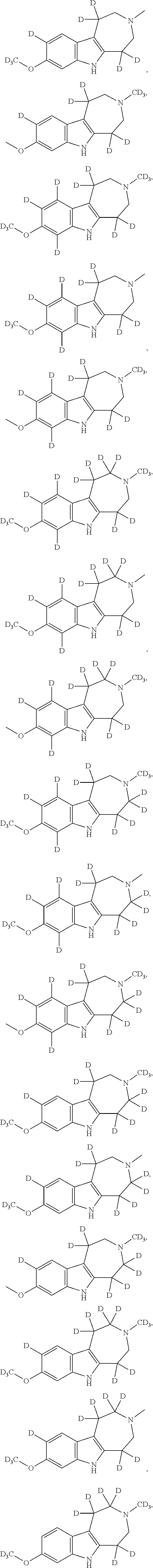 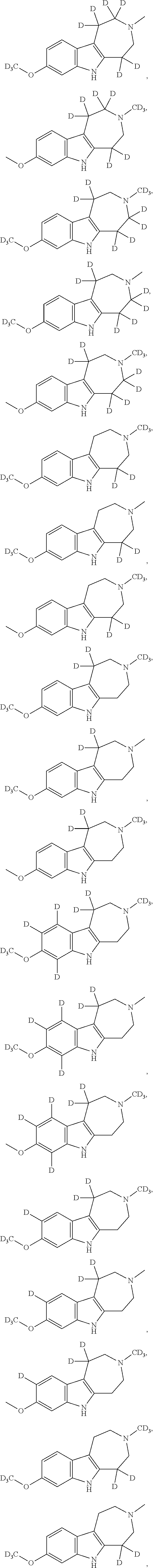 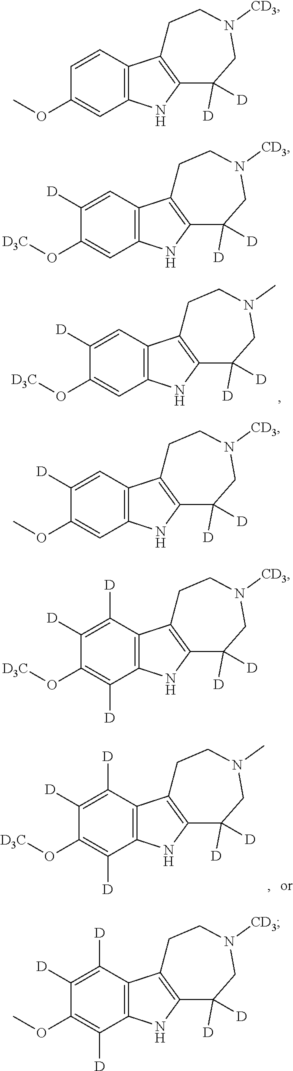
or a pharmaceutically acceptable salt thereof.

115. A pharmaceutically acceptable salt of the compound of claim 103.

116. A pharmaceutically acceptable salt of the compound of claim 114.

117. A pharmaceutical composition comprising the compound of claim 1, or a pharmaceutically acceptable salt thereof.

118. A pharmaceutical composition comprising the compound of claim 114, or a pharmaceutically acceptable salt thereof.
